(19) United States
(12) Patent Application Publication
(10) Pub. No.: US 2024/0148849 A1
(43) Pub. Date: May 9, 2024

(54) IMMUNOGENIC COMPOSITION, USE AND METHODS

(71) Applicant: GLAXOSMITHKLINE BIOLOGICALS SA, Rixensart (BE)

(72) Inventors: Ashwani ARORA, Siena (IT); Daniela CASULA, Siena (IT); Mario CONTORNI, Siena (IT); Fernando ULLOA-MONTOYA, Rixensart (BE); Simona RONDINI, Siena (IT); Tian SUN, Rixensart (BE); Marco TESTA, Siena (IT)

(73) Assignee: GLAXOSMITHKLINE BIOLOGICALS SA, Rixensart (BE)

(21) Appl. No.: 18/277,843

(22) PCT Filed: Feb. 18, 2022
(86) PCT No.: PCT/EP2022/054029

§ 371 Date: Aug. 18, 2023

(30) Foreign Application Priority Data
Feb. 22, 2021 (EP) 21158503.9
Mar. 19, 2021 (EP) 21163667.5

(51) Int. Cl. A61K 39/102; A61K 39/104; A61P 11/00

(52) U.S. Cl. CPC A61K 39/102; A61K 39/1045; A61P 11/00; A61K 2039/55572

(57) ABSTRACT

The present invention relates to the field of immunogenic compositions and the use of such compositions in medicine. More particularly, it relates to immunogenic compositions comprising an immunogenic polypeptide from non-typeable Haemophilus influenzae, a PE-PilA fusion protein, and optionally an immunogenic polypeptide of UspA2 from Moraxella catarrhalis, for use in subjects having chronic obstructive pulmonary disease (COPD), in particular for reducing the frequency of severe exacerbations (i.e. severe AECOPDs).

### TECHNICAL FIELD

The present invention relates to the field of immunogenic compositions and the use of such compositions in medicine. More particularly, it relates to immunogenic compositions comprising an immunogenic polypeptide from non-typeable Haemophilus influenzae, a PE-PilA fusion protein, and optionally an immunogenic polypeptide of UspA2 from Moraxella catarrhalis, for use in subjects having chronic obstructive pulmonary disease (COPD), in particular for reducing the frequency of severe exacerbations (i.e. severe AECOPDs).

### BACKGROUND TO THE INVENTION

Chronic Obstructive Pulmonary Disease (COPD) is a chronic inflammatory disorder resulting in irreversible decline in lung function as a consequence of inhalation of tobacco smoke or other irritants. Chronic obstructive pulmonary disease (COPD) is recognised as encompassing several conditions (airflow obstruction, chronic bronchitis, bronchiolitis or small airways disease and emphysema) that often coexist (Wilson et al., Eur. Respir. J. 2001; 17: 995-1007). Patients suffer exacerbations of COPD that are usually associated with increased breathlessness, and often have increased cough that may be productive of mucus or purulent sputum (Wilson, Eur Respir J 2001 17:995-1007). COPD is defined physiologically by the presence of irreversible or partially reversible airway obstruction in patients with chronic bronchitis and/or emphysema (Standards for the diagnosis and care of patients with chronic obstructive pulmonary disease. American Thoracic Society. Am J Respir Crit Care Med. 1995 November; 152(5 Pt 2):577-121).

Chronic obstructive pulmonary disease (COPD) is a major cause of morbidity and mortality. Chronic Obstructive Pulmonary Disease is one of the leading causes of death in the world (Lancet 2018, 392:1736-88; Lancet 2012, 380: 2095-128). The course of COPD is characterized by progressive worsening of airflow limitation and a decline in pulmonary function. COPD may be complicated by frequent and recurrent acute exacerbations (AE), which are associated with enormous health care expenditure and high morbidity. (Proceedings of the American Thoracic Society 4:554-564 (2007)). One study suggests that approximately 50% of acute exacerbations of symptoms in COPD are caused by non-typeable Haemophilus influenzae, Moraxella catarrhalis, Streptococcus pneumoniae, and Pseudomonas aeruginosa. (Drugs and Aging 26:985-999 (2009)). Haemophilus influenzae (H. influenzae) is found in 20-30% of exacerbations of COPD; Streptococcus pneumoniae, in 10-15% of exacerbations of COPD; and Moraxella catarrhalis, in 10-15% of exacerbations of COPD. (New England Journal of Medicine 359:2355-2365 (2008)). Haemophilus influenzae, Streptococcus pneumoniae, and Moraxella catarrhalis have been shown to be the primary pathogens in acute exacerbations of bronchitis in Hong Kong, South Korea, and the Phillipines, while Klebsiella spp., Pseudomonas aeruginosa and Acinetobacter spp. constitute a large proportion of pathogens in other Asian countries/regions including Indonesia, Thailand, Malaysia and Taiwan (Respirology, (2011) 16, 532-539; doi:10.1111/j.1440.1843.2011.01943.x). In Bangladesh, 20% of patients with COPD showed positive sputum culture for Pseudomonas, Klebsiella, Streptococcus pneumoniae and Haemophilus influenzae, while 65% of patients with AECOPD (acute exacerbation of COPD) showed positive cultures for Pseudomonas, Klebsiella, Acinetobacter, Enterobacter, Moraxella catarrhalis and combinations thereof. (Mymensingh Medical Journal 19:576-585 (2010)). However, it has been suggested that the two most important measures to prevent COPD exacerbation are active immunizations and chronic maintenance of pharmacotherapy. (Proceedings of the American Thoracic Society 4:554-564 (2007)).

One of the difficulties in treating and managing COPD is the heterogeneity of this complex disease in terms of severity, progression, exercise tolerance, and nature of symptoms. This complexity is also evident in acute exacerbations of COPD (AECOPD), which are transient and apparently stochastic periods of increased COPD symptoms requiring additional medical treatment and often hospitalization (Sethi et al., N Eng J Med 2008; 359:2355-65). Known subtypes of exacerbations are defined by the nature of key triggers including bacterial or viral infections, and/or high eosinophil levels, and these events are typically treated with a combination of antibiotics and steroids in a non-specific manner (Bafadhel et al., Am J Respir Crit Care Med 2011; 184:662).

There exists a need for improved uses of immunogenic compositions and methods for the prevention and/or treatment of acute exacerbations of COPD (AECOPD).

### SUMMARY OF THE INVENTION

According to the present invention, it has been found that an immunogenic composition comprising: (i) immunogenic polypeptide from non-typeable Haemophilus influenzae, (ii) a PE-PilA fusion protein, and (iii) optionally an immunogenic polypeptide of UspA2 from Moraxella catarrhalis, can be used in the treatment or prevention of chronic obstructive pulmonary disease (COPD) in a subject (e.g. human) for reducing the frequency of severe exacerbations (severe AECOPDs). This may be particularly useful in specific subsets of subjects suffering from COPD. For example, this may be useful where the subject has one of more of the following: (i) the subject has GOLD 4 (very severe) COPD status, (ii) the subject has experienced at least 2 moderate AECOPD or at least one severe AECOPD in the previous 12 months, (iii) the subject is taking ICS as a maintenance therapy, and (iv) the subject has received a pneumococcal vaccine. These findings can be used to determine appropriate treatments for a given subject (e.g. a COPD patient).

Accordingly, there is provided in one aspect of the invention an immunogenic composition comprising: (i) an immunogenic polypeptide from non-typeable Haemophilus influenzae, (ii) a PE-PilA fusion protein, (iii) optionally an immunogenic polypeptide of UspA2 from Moraxella catarrhalis, and (iv) an adjuvant, for use in reducing the frequency of severe exacerbations, in a subject (e.g. human) having chronic obstructive pulmonary disease (COPD).

There is provided the use of an immunogenic composition comprising: (i) an immunogenic polypeptide from non-typeable Haemophilus influenzae, (ii) a PE-PilA fusion protein, (iii) optionally an immunogenic polypeptide of UspA2 from Moraxella catarrhalis, and (iv) an adjuvant, in the manufacture of a medicament for reducing the frequency of severe exacerbations, in a subject (e.g. human) having chronic obstructive pulmonary disease (COPD).

There is provided a method of treatment or prevention of chronic obstructive pulmonary disease (COPD) by reducing the frequency of severe exacerbations, in a subject (e.g. human) having chronic obstructive pulmonary disease (COPD), said method comprising administering to said subject a therapeutically effective amount of an immunogenic composition comprising: (i) an immunogenic polypeptide from non-typeable Haemophilus influenzae, (ii) a PE-PilA fusion protein, (iii) optionally an immunogenic polypeptide of UspA2 from Moraxella catarrhalis, and (iv) an adjuvant. There is also provided a method of treatment of chronic obstructive pulmonary disease (COPD) by reducing the frequency of severe exacerbations, in a subject (e.g. human) having chronic obstructive pulmonary disease (COPD), said method comprising administering to said subject a therapeutically effective amount of an immunogenic composition comprising: (i) an immunogenic polypeptide from non-typeable Haemophilus influenzae, (ii) a PE-PilA fusion protein, (iii) optionally an immunogenic polypeptide of UspA2 from Moraxella catarrhalis, and (iv) an adjuvant. There is also provided a method of reducing the frequency of severe exacerbations, in a subject (e.g. human) having chronic obstructive pulmonary disease (COPD), said method comprising administering to said subject a therapeutically effective amount of an immunogenic composition comprising: (i) an immunogenic polypeptide from non-typeable Haemophilus influenzae, (ii) a PE-PilA fusion protein, (iii) optionally an immunogenic polypeptide of UspA2 from Moraxella catarrhalis, and (iv) an adjuvant.

### DETAILED DESCRIPTION

### Terminology

A “subject” as used herein is a mammal, including humans, non-human primates, and non-primate mammals such as members of the rodent genus (including but not limited to mice and rats) and members of the order Lagomorpha (including but not limited to rabbits). In one embodiment, the subject is a human.

As used herein, “adjuvant” means a compound or substance that, when administered to a subject in conjunction with a vaccine, immunotherapeutic, or other antigen- or immunogen-containing composition, increases or enhances the subject's immune response to the administered antigen or immunogen (as compared to the immune response that would be obtained in the absence of adjuvant).

As used herein, the term “immunogenic polypeptide” is a polypeptide or fragment of a polypeptide (i.e. an immunogenic fragment), that is capable of eliciting a humoral and/or cellular immune response in a host animal, e.g. human, specific for that polypeptide. Immunogenic polypeptides can be produced using techniques known in the art, e.g. recombinantly, by proteolytic digestion, or by chemical synthesis. Immunogenic polypeptides may be derived from an amino acid sequence at least 70%, 80%, 85%, 90%, 91%, 92%, 93%, 94%, 95%, 96%, 97%, 98%, 99%, or 100% identical to a reference sequence (e.g. SEQ ID NO: 1 to 21 as described herein) which has been modified by the deletion and/or addition and/or substitution of one or more amino acids (e.g. 1, 2, 3, 4, 5, 6, 7, 8, 9, 10, 11 or 12 amino acids). Amino acid substitution may be conservative or non-conservative. In one aspect, amino acid substitution is conservative. Substitutions, deletions, additions or any combination thereof may be combined in a single variant so long as the variant is an immunogenic polypeptide. In an embodiment, an immunogenic polypeptide is a recombinant polypeptide.

As used herein, the term “immunogenic fragment” is a fragment of a polypeptide, that is capable of eliciting a humoral and/or cellular immune response in a host animal, e.g. human, specific for that polypeptide. Fragments of a polypeptide can be produced using techniques known in the art, e.g. recombinantly, by proteolytic digestion, or by chemical synthesis. Internal or terminal fragments of a polypeptide can be generated by removing one or more nucleotides from one end (for a terminal fragment) or both ends (for an internal fragment) of a nucleic acid which encodes the polypeptide. Typically, fragments comprise at least 10, 20, 30, 40 or 50 contiguous amino acids of the full length sequence. Fragments may be readily modified by adding or removing 1, 2, 3, 4, 5, 6, 7, 8, 9, 10, 20, 30, 40 or 50 amino acids from either or both of the N and C termini.

As used herein, the term “conservative amino acid substitution” involves substitution of a native amino acid residue with a non-native residue such that there is little or no effect on the size, polarity, charge, hydrophobicity, or hydrophilicity of the amino acid residue at that position, and without resulting in decreased immunogenicity. For example, these may be substitutions within the following groups: valine, glycine; glycine, alanine; valine, isoleucine, leucine; aspartic acid, glutamic acid; asparagine, glutamine; serine, threonine; lysine, arginine; and phenylalanine, tyrosine. Conservative amino acid modifications to the sequence of a polypeptide (and the corresponding modifications to the encoding nucleotides) may produce polypeptides having functional and chemical characteristics similar to those of a reference polypeptide.

As used herein “signal peptide” refers to a short (less than 60 amino acids, for example, 3 to 60 amino acids) polypeptide present on precursor proteins (typically at the N terminus), and which is typically absent from the mature protein. The signal peptide (sp) is typically rich in hydrophobic amino acids. The signal peptide directs the transport and/or secretion of the translated protein through the membrane. Signal peptides may also be called targeting signals, transit peptides, localization signals, or signal sequences. For example, the signal sequence may be a co-translational or post-translational signal peptide.

As used herein, the term “deletion” is the removal of one or more amino acid residues from the polypeptide sequence. Typically, no more than about from 1 to 6 residues (e.g. 1 to 4 residues) are deleted at any one site within the protein molecule.

As used herein, the terms “insertion” or “addition” (including other tenses thereof such as “inserted”) means the addition of one or more non-native amino acid residues in the polypeptide sequence. Typically, no more than about from 1 to 10 residues, (e.g. 1 to 7 residues, 1 to 6 residues, or 1 to 4 residues) are inserted at any one site within the polypeptide molecule.

The terms “identical” or percent “identity” refer to amino acid sequences that are the same or have a specified percentage of amino acid residues that are the same (e.g. 80%, 85%, 90%, 92%, 95%, 96%, 97%, 98% or 99% identity over a specified region), when compared and aligned for maximum correspondence using, for example, sequence comparison algorithms or by manual alignment and visual inspection. In general, when calculating percentage identity the two sequences to be compared are aligned to give a maximum correlation between the sequences. Identity between polypeptides may be calculated by various algorithms. The Gap and Needle programs are an implementation of the Needleman-Wunsch algorithm described in: Needleman, S. B. and Wunsch, C. D. (1970) J. Mol. Biol. 48, 443-453. These programs are using frequently the BLOSUM62 scoring matrix (Steven Henikoft and Jorja G. Henikoft (1992), “Amino acid substitution matrices from protein blocks”), Proc. Natl. Acad. Sci. USA 89 (Biochemistry): 10915-10919) with gap open and extension penalties of, respectively, 8 and 2. As used herein, identity is calculated using the Needle program, from the EMBOSS package (Free software; EMBOSS: The European Molecular Biology Open Software Suite) or using the algorithm described by Dufresne et al. in Nature Biotechnology in 2002 (vol. 20, pp. 1269-71) used in the software GenePAST (Genome Quest Life Sciences, Inc. Boston, M A, 2021). The GenePAST “percent identity” algorithm finds the best fit between the query sequence and the subject sequence, and expresses the alignment as an exact percentage. GenePAST makes no alignment scoring adjustments based on considerations of biological relevance between query and subject sequences. Identity between two sequences is calculated across the entire length of both sequences and is expressed as a percentage of the reference sequence (e.g. SEQ ID NO: 1 to 21 as described herein).

As used herein the term “recombinant” means artificial or synthetic. In certain embodiments, a “recombinant protein” or a “recombinant polypeptide” refers to a protein or polypeptide that has been made using recombinant nucleotide sequences (nucleotide sequences introduced into a host cell). In certain embodiments, the nucleotide sequence that encodes a “recombinant protein” or “recombinant polypeptide” is heterologous to the host cell.

As used herein the terms “isolated” or “purified” mean a polypeptide in a form not found in nature. This includes, for example, a polypeptide having been separated from host cell or organism (including crude extracts) or otherwise removed from its natural environment. In certain embodiments, an isolated or purified polypeptide essentially free from all other polypeptides with which the protein is innately associated (or innately in contact with).

As used herein, the term “acute exacerbation of COPD” or “AECOPD” is an event characterised by a worsening of the patient's respiratory symptoms that is beyond normal day-to-day variations. Typically, an AECOPD leads to a change in medication.

As used herein, the term “severe exacerbation” or “severe exacerbation of COPD” or “severe AECOPD” or “severe episode” (which terms may be used interchangeably) is an AECOPD that requires hospitalization. A severe acute exacerbation of COPD is any sustained increase in respiratory symptomatology compared with the baseline situation requiring modification of regular medication and hospital treatment. The subject has been admitted at the hospital for observation and/or treatment for AECOPD that would not have been appropriate in the physician's office or in an outpatient setting. This is in contrast to mild exacerbations which can be controlled with an increase in dosage of regular medications, and to moderate exacerbations which are treated with systemic corticosteroids and/or antibiotics, both of which can be treated without hospitalization.

As used herein, the term “therapeutically effective amount” is at least the minimum concentration required to effect a measurable improvement of a particular disorder. A therapeutically effective amount herein may vary according to factors such as the disease state, age, sex, and weight of the patient, and the ability of the antibody to elicit a desired response in the individual. A therapeutically effective amount is also one in which any toxic or detrimental effects of the antibody are outweighed by the therapeutically beneficial effects.

As used herein, the term “treatment of an COPD” means ameliorating, stabilising, reducing or eliminating the increased symptoms that are a feature of an exacerbation in a subject.

As used herein, the term “prevention of an COPD” means preventing, reducing the incidence or frequency, or reducing the severity of acute exacerbations of chronic obstructive pulmonary disease (AECOPD) in a subject. In one embodiment, an immunogenic composition according to the present invention is for the reduction of the frequency of severe exacerbations of chronic obstructive pulmonary disease (COPD) in a subject. In another embodiment, an immunogenic composition according to the present invention is for the reduction of the frequency of severe exacerbations of chronic obstructive pulmonary disease (COPD) resulting from a bacterial infection in a subject.

As used herein, the term “bacterial exacerbation” refers to an exacerbation associated with a positive bacterial pathogen on routine culture (Haemophilus influenza, Moraxella catarrhalis, Streptococcus pneumoniae, Staphylococcus aureus or Pseudomonas aeruginosa) or a total aerobic CFU count greater than or equal to 10⁷ cells. In one embodiment, the bacterial exacerbation is associated with a positive bacterial culture for
a) Haemophilus influenzae (e.g. non-typeable H. influenzae (NTHi));
b) Moraxella catarrhalis; or
c) Haemophilus influenzae (e.g. non-typeable H. influenzae (NTHi)) and Moraxella catarrhalis.

In another embodiment, the bacterial exacerbation is associated with a positive bacterial culture for Haemophilus influenzae (e.g. non-typeable H. influenzae (NTHi)).

As used herein, the term “corticosteroid” means a synthetic pharmaceutical medicament that mimics the action of naturally occurring corticosteroids. Corticosteroids are most often used to treat diseases of immunity and inflammation. Examples of oral corticosteroids include bethamethasone, prednisone, prednisolone, triamcinolone, methylprednisolone, dexamethasone and fludrocortisone. Systemic oral and injectable corticosteroids include glucocorticoids (e.g. hydrocortisone, cortisone, ethamethasoneb, prednisone, prednisolone, triamcinolone and dexamethasone) or mineralocorticoids (e.g. fludrocortisone). Examples of inhaled corticosteroids (ICS) include beclomethasone dipropionate, budesonide, ciclesonide, flunisolide, fluticasone propionate, fluticasone furoate, or mometasone furoate. Oral and injectable corticosteroids act systemically and are referred to herein as “systemic corticosteroids”, distinguishing them from inhaled corticosteroids, which act topically.

As used herein, the term “lung function” refers to a COPD patient's forced expiratory volume in 1 second (FEV₁). FEV₁ is the volume of air exhaled during the first second of maximal forced expiration starting from a position of full inspiration.

Chronic obstructive pulmonary disease (COPD) is a lung disease characterized by chronic obstruction of lung airflow that interferes with normal breathing and is not fully reversible. A COPD diagnosis is confirmed by a simple test called spirometry, which measures how deeply a person can breathe and how fast air can move into and out of the lungs. Such a diagnosis should be considered in any patient who has symptoms of cough, sputum production, or dyspnea (difficult or labored breathing), and/or a history of exposure to risk factors for the disease. Where spirometry is unavailable, the diagnosis of COPD should be made using all available tools. Clinical symptoms and signs, such as abnormal shortness of breath and increased forced expiratory time, can be used to help with the diagnosis. A low peak flow is consistent with COPD, but may not be specific to COPD because it can be caused by other lung diseases and by poor performance during testing. Chronic cough and sputum production often precede the development of airflow limitation by many years, although not all individuals with cough and sputum production go on to develop COPD.

A reduction in frequency, duration or severity of acute exacerbation or one or more symptoms of an exacerbation may be measured by clinical observation by a doctor or clinician. A reduction in frequency, duration or severity is determined relative to the frequency, duration or severity of an exacerbation or symptom in the same subject not treated according to the methods of the present invention. Suitable clinical observations by an ordinarily skilled clinician may include objective measures of lung function, as well as the frequency with which medical intervention is required. Subjective self-evaluation by the subject may also be used as a measure, for example, using an FDA-recognized subject reported outcome tool or the Exacerbations from Pulmonary Disease Tool (EXACT-PRO).

### DESCRIPTION OF FIGURES

FIG. 1: ABCD Assessment Tool for COPD

COPD is characterised by progressive worsening of airflow limitation and a decline in pulmonary function. The disease is complicated by acute exacerbations (AECOPD), which are transient and apparently stochastic periods of increased COPD symptoms requiring additional medical treatment and often hospitalization.

FIG. 2: Forest plot of vaccine efficacy for moderate and severe AECOPD in each subgroup analysis in one-year period starting one month post-dose 2 (modified total vaccinated cohort). Subgroups are of status at baseline, except where indicated. Exacerbation history is number of moderate and severe exacerbations in previous 12 months and frequent exacerbators had ≥2 moderate or severe exacerbations or ≥1 severe exacerbation in previous 12 months. CI, confidence interval; GOLD, Global Initiative for Chronic Obstructive Lung Disease; ICS, inhaled corticosteroid.

FIG. 3: Bar chart showing hospitalizations due to AECOPD from 1 m post dose 2 by Season.

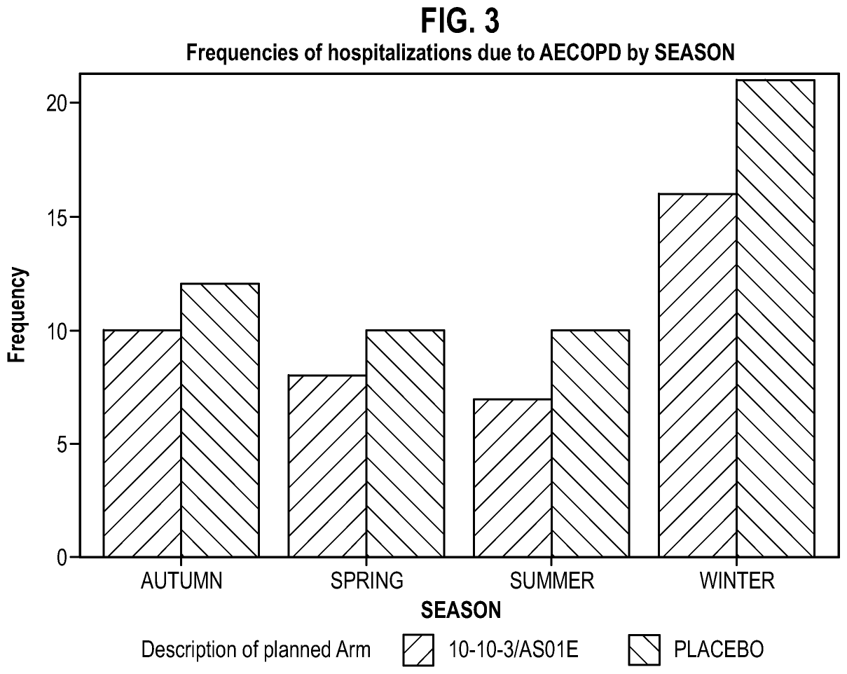

There is provided in one aspect of the present invention an immunogenic composition comprising: (i) an immunogenic polypeptide from non-typeable Haemophilus influenzae, (ii) a PE-PilA fusion protein, (iii) an immunogenic polypeptide of UspA2 from Moraxella catarrhalis, and (iv) an adjuvant, for use in reducing the frequency of severe exacerbations, in a subject (e.g. human) having chronic obstructive pulmonary disease (COPD).

There is provided the use of an immunogenic composition comprising: (i) an immunogenic polypeptide from non-typeable Haemophilus influenzae, (ii) a PE-PilA fusion protein, (iii) an immunogenic polypeptide of UspA2 from Moraxella catarrhalis, and (iv) an adjuvant, in the manufacture of a medicament for reducing the frequency of severe exacerbations, in a subject (e.g. human) having chronic obstructive pulmonary disease (COPD).

There is provided a method of treatment or prevention of chronic obstructive pulmonary disease (COPD) by reducing the frequency of severe exacerbations, in a subject (e.g. human) having chronic obstructive pulmonary disease (COPD), said method comprising administering to said subject a therapeutically effective amount of an immunogenic composition comprising: (i) an immunogenic polypeptide from non-typeable Haemophilus influenzae, (ii) a PE-PilA fusion protein, (iii) an immunogenic polypeptide of UspA2 from Moraxella catarrhalis, and (iv) an adjuvant. There is also provided a method of treatment of chronic obstructive pulmonary disease (COPD) by reducing the frequency of severe exacerbations, in a subject (e.g. human) having chronic obstructive pulmonary disease (COPD), said method comprising administering to said subject a therapeutically effective amount of an immunogenic composition comprising: (i) an immunogenic polypeptide from non-typeable Haemophilus influenzae, (ii) a PE-PilA fusion protein, (iii) an immunogenic polypeptide of UspA2 from Moraxella catarrhalis, and (iv) an adjuvant. There is also provided a method of reducing the frequency of severe exacerbations, in a subject (e.g. human) having chronic obstructive pulmonary disease (COPD), said method comprising administering to said subject a therapeutically effective amount of an immunogenic composition comprising: (i) an immunogenic polypeptide from non-typeable Haemophilus influenzae, (ii) a PE-PilA fusion protein, (iii) an immunogenic polypeptide of UspA2 from Moraxella catarrhalis, and (iv) an adjuvant.

### Reducing the Frequency of Severe Exacerbations

The present inventors have found that immunogenic compositions of the invention can be used to reduce the frequency of severe exacerbations (and thus hospitalization visits due to COPD), in a subject (e.g. human) having chronic obstructive pulmonary disease (COPD), see Table 6 in the Examples. The frequency of exacerbations may be measured in terms of yearly rate, the average number of episodes of AECOPD in a year. The immunogenic compositions of the invention may be used to reduce the yearly rate of severe exacerbations. The immunogenic compositions may be used to reduce the yearly rate of severe exacerbations, for example by at least 5%, suitably 5%, 10%, 15%, 20%, 25% 30%, 35% or 36% in a subject that has been administered the immunogenic composition compared to a subject who has not been administered the immunogenic composition. The immunogenic compositions may be used to reduce the yearly rate of severe exacerbations, for example by up to 62%, in a subject that has been administered the immunogenic composition compared to a subject who has not been administered the immunogenic composition. In particular, the immunogenic compositions may be used to reduce the yearly rate of severe exacerbations, e.g. by 36%, in a subject that has been administered the immunogenic composition compared to a subject who has not been administered the immunogenic composition. This may be useful in subjects who have experienced severe exacerbations. Thus the present invention provides an immunogenic composition for use, use of an immunogenic composition or method according to the present invention, wherein the subject (e.g. a human aged 40 to 80 years old) has experienced at least one severe episode of AECOPD within a period of 12 months (e.g. in the previous 12 months). The present invention also provides an immunogenic composition for use, use of an immunogenic composition or method according to the present invention, wherein the subject (e.g. a human aged 40 to 80 years old) has experienced at least one moderate episode of acute exacerbation in chronic obstructive pulmonary disease (AECOPD) or at least one severe episode of AECOPD within a period of 12 months (e.g. in the previous 12 months). The present invention also provides an immunogenic composition for use, use of an immunogenic composition or method according to the present invention, wherein the subject (e.g. a human aged 40 to 80 years old) has experienced at least 2 moderate episodes of acute exacerbation in chronic obstructive pulmonary disease (AECOPD) or at least one severe episode of AECOPD within a period of 12 months (e.g. in the previous 12 months).

The present invention may be used to reduce the likelihood of hospitalization due to an AECOPD in a subject having COPD. Furthermore, the present invention may be used to reduce the frequency of pneumonia due to COPD (e.g. due to an AECOPD) in a subject (e.g. human) having chronic obstructive pulmonary disease (COPD). The present invention may be used to reduce the need for intensive care and/or mechanical ventilation due to COPD (e.g. due to an AECOPD) in a subject having COPD. The present invention may also be used to reduce the likelihood of mortality due to an AECOPD in a subject having COPD.

As described in the Examples (Table 8), a reduction in frequency of severe exacerbations was observed in subjects that are “frequent exacerbators” having experienced at least 2 moderate AECOPD or at least one severe AECOPD in the previous 12 months. Thus the immunogenic composition may be useful to reduce the frequency of severe exacerbations, in a subject (e.g. human) that have experienced at least 2 moderate AECOPD or at least one severe AECOPD in the previous 12 months. The immunogenic composition of the present invention may be useful in a subject (e.g. human) that has experienced at least 2 moderate AECOPD or at least one severe AECOPD in the previous 12 months to reduce the frequency of severe exacerbations of COPD.

The present invention also provides an immunogenic composition for use, use of an immunogenic composition or method according to the invention, wherein the subject has experienced at least 2 moderate AECOPD or at least one severe AECOPD in the previous 12 months for reducing the yearly rate of severe exacerbations, e.g. by at least 13% (e.g. up to 74%), compared to a subject who is not administered the immunogenic composition. The immunogenic compositions may be used to reduce the yearly rate of severe exacerbations wherein the subject has experienced at least 2 moderate AECOPD or at least one severe AECOPD in the previous 12 months, e.g. by 52%, in a subject that has been administered the immunogenic composition compared to a subject who has not been administered the immunogenic composition. The immunogenic compositions may be used to reduce the yearly rate of severe exacerbations wherein the subject has experienced at least 2 moderate AECOPD or at least one severe AECOPD in the previous 12 months by at least 5%, for example by 10%, 20%, 30%, 40%, 50% or 52% in a subject that has been administered the immunogenic composition compared to a subject who has not been administered the immunogenic composition. The present invention also provides the immunogenic composition for use, use of an immunogenic composition or method for reducing the frequency of severe exacerbations, in a subject having COPD comprising: (a) selecting a subject who has experienced at least 2 moderate AECOPD or at least one severe AECOPD in the previous 12 months, and (b) administering to the subject the immunogenic composition.

### Particular Aspects of the Present Invention Include:

An immunogenic composition comprising: (i) an immunogenic polypeptide from non-typeable Haemophilus influenzae, (ii) a PE-PilA fusion protein, (iii) optionally an immunogenic polypeptide of UspA2 from Moraxella catarrhalis, and (iv) an adjuvant, for use in reducing the frequency of severe exacerbations, in a subject (e.g. human) having chronic obstructive pulmonary disease (COPD).
The use of an immunogenic composition comprising: (i) an immunogenic polypeptide from non-typeable Haemophilus influenzae, (ii) a PE-PilA fusion protein, (iii) optionally an immunogenic polypeptide of UspA2 from Moraxella catarrhalis, and (iv) an adjuvant, in the manufacture of a medicament for reducing the frequency of severe exacerbations, in a subject (e.g. human) having chronic obstructive pulmonary disease (COPD).
A method of reducing the frequency of severe exacerbations, in a subject (e.g. human) having chronic obstructive pulmonary disease (COPD), said method comprising administering to said subject a therapeutically effective amount of an immunogenic composition comprising: (i) an immunogenic polypeptide from non-typeable Haemophilus influenzae, (ii) a PE-PilA fusion protein, (iii) optionally an immunogenic polypeptide of UspA2 from Moraxella catarrhalis, and (iv) an adjuvant.
The immunogenic composition for use, use of an immunogenic composition or method as described above, for reducing the yearly rate of severe exacerbations, compared to a subject who has not been administered the immunogenic composition.
The immunogenic composition for use, use of an immunogenic composition or method as described above, for reducing the likelihood of hospitalization due to an AECOPD in a subject (e.g. human) having chronic obstructive pulmonary disease (COPD).
The immunogenic composition for use, use of an immunogenic composition or method as described above, for reducing the frequency of pneumonia due to COPD (e.g. due to an AECOPD) in a subject (e.g. human) having chronic obstructive pulmonary disease (COPD).
The immunogenic composition for use, use of an immunogenic composition or method as described above, for reducing the need for intensive care and/or mechanical ventilation due to COPD (e.g. due to an AECOPD) in a subject having COPD.
The immunogenic composition for use, use of an immunogenic composition or method as described above, for reducing the likelihood of mortality due to an AECOPD in a subject having COPD.
The immunogenic composition for use, use of an immunogenic composition or method as described above, wherein the subject has experienced at least 2 moderate AECOPD or at least one severe AECOPD in the previous 12 months.
The immunogenic composition for use, use of an immunogenic composition or method as described above, wherein the subject has experienced at least one severe AECOPD in the previous 12 months.
The immunogenic composition for use, use of an immunogenic composition or method as described above, wherein the immunogenic polypeptide from non-typeable Haemophilus influenzae is an immunogenic polypeptide of Protein D, suitably an isolated immunogenic polypeptide with at least 70%, 80%, 85%, 90%, 91%, 92%, 93%, 94%, 95%, 96%, 97%, 98%, 99%, or 100% to SEQ ID NO: 2.
The immunogenic composition for use, use of an immunogenic composition or method as described above, wherein the PE-PilA fusion protein is an isolated immunogenic polypeptide with at least 70%, 80%, 85%, 90%, 91%, 92%, 93%, 94%, 95%, 96%, 97%, 98%, 99%, or 100% to SEQ ID NO: 9.
The immunogenic composition for use, use of an immunogenic composition or method as described above, wherein the composition comprises an immunogenic polypeptide of UspA2 from Moraxella catarrhalis, suitably an isolated immunogenic polypeptide with at least 70%, 80%, 85%, 90%, 91%, 92%, 93%, 94%, 95%, 96%, 97%, 98%, 99%, or 100% to a polypeptide selected from the group consisting of MC-001 (SEQ ID NO: 11), MC-002 (SEQ ID NO: 12), MC-003 (SEQ ID NO: 13), MC-004 (SEQ ID NO: 14), MC-005 (SEQ ID NO: 15), MC-006 (SEQ ID NO: 16), MC-007 (SEQ ID NO: 17), MC-008 (SEQ ID NO:18), MC-009 (SEQ ID NO: 19), MC-010 (SEQ ID NO: 20) or MC-011 (SEQ ID NO: 21) e.g. MC009 SEQ ID NO: 19.
The immunogenic composition for use, use of an immunogenic composition or method as described above, wherein the immunogenic composition comprises a pharmaceutically acceptable excipient or carrier.
The immunogenic composition for use, use of an immunogenic composition or method as described above, wherein the adjuvant is an AS01 adjuvant, e.g. ASO1E.
   Use/Methods of Treatment in Subjects having GOLD 4 (Very Severe) COPD

In an embodiment the subject is an animal, preferably a mammal, including humans. In a preferred embodiment the subject is a human. As used herein, the term “subject” may be used interchangeably with the word “patient”, i.e. in an embodiment the subject is a patient.

In an embodiment, the subject may be an adult human, for example, aged between 18 and 40, or between 50 and 70, or between 40 and 85 years of age. In an embodiment, the subject is a human aged between 40 and 80 years of age. The subject has a previous history of Chronic Obstructive Pulmonary Disease (COPD), particularly, a previous history of moderate and severe Acute Exacerbation of Chronic Obstructive Pulmonary Disease (AECOPD). For example, a confirmed diagnosis of COPD, categorised as moderate, severe, or very severe according to the Global Initiative for Chronic Obstructive Lung Disease (GOLD) classification. The Global Strategy for the Diagnosis, Management and Prevention of COPD prepared by GOLD state that COPD should be considered in any patient with dyspnea, chronic cough or sputum production, and/or a history of exposure to risk factors for the disease, such as tobacco smoking, occupation, or pollutants. A spirometry assessment, measuring airflow limitation, is required to establish diagnosis. The classification of airflow limitation severity in COPD outlined in the GOLD strategy is shown in Table 1.

TABLE 1
Classification of airflow limitation severity in COPD (Based
on post-bronchodilator FEV₁) In patients with FEV₁/FVC < 0.70

| | | |
|---|---|---|
| GOLD 1 | Mild | FEV₁ ≥ 80% predicted |
| GOLD 2 | Moderate | 50% ≤ FEV₁ < 80% predicted |
| GOLD 3 | Severe | 30% ≤ FEV₁ < 50% predicted |
| GOLD 4 | Very Severe | FEV₁ < 30% predicted |

COPD assessment also includes analysis of patient symptoms, and this can be performed using comprehensive disease-specific health status questionnaires such as the Chronic Respiratory Questionnaire (CRQ) and St. George's Respiratory Questionnaire (SGRQ). For routine practice the COPD Assessment Test (CAT™) and The COPD Control Questionnaire (The CCQ© have been developed. The CAT™ and CCQ© tests do not categorise patients for the purpose of treatment, however for the SRGQ assessment a symptom score ≥25 may be used as the threshold for considered regular treatment for breathlessness. The equivalent threshold for the CAT™ is 10. A simple assessment of breathlessness is the Modified British Medical Research Council (mMRC) Questionnaire.

According to the GOLD strategy, of the patients classified at the GOLD 2 (moderate) stage, approximately 20% may experience frequent exacerbations requiring antibiotic and/or systemic corticosteroid therapy in addition to regular maintenance therapy. The risk of exacerbations is significantly higher for patients classified as GOLD 3 (severe) and GOLD 4 (very severe). In an embodiment, the subject has a confirmed diagnosis of COPD (based on post-bronchodilator spirometry) with forced expiratory volume in 1 second (FEV₁) over forced vital capacity (FVC) ratio (FEV₁/FVC)<0.7, AND FEV₁<80% predicted. In an embodiment, the subject has GOLD 2 (moderate), GOLD 3 (severe) or GOLD 4 (very severe) status. In another embodiment, the subject has a confirmed diagnosis of COPD (based on post-bronchodilator spirometry) with forced expiratory volume in 1 second (FEV₁) over forced vital capacity (FVC) ratio (FEV₁/FVC)<0.7, AND FEV₁<50% predicted. In another embodiment, the subject has GOLD 3 (severe) or GOLD 4 (very severe) status. In another embodiment, the subject has a confirmed diagnosis of COPD (based on post-bronchodilator spirometry) with forced expiratory volume in 1 second (FEV₁) over forced vital capacity (FVC) ratio (FEV₁/FVC)<0.7, AND FEV₁<30% predicted. In another embodiment, the subject has GOLD 4 (very severe) status,

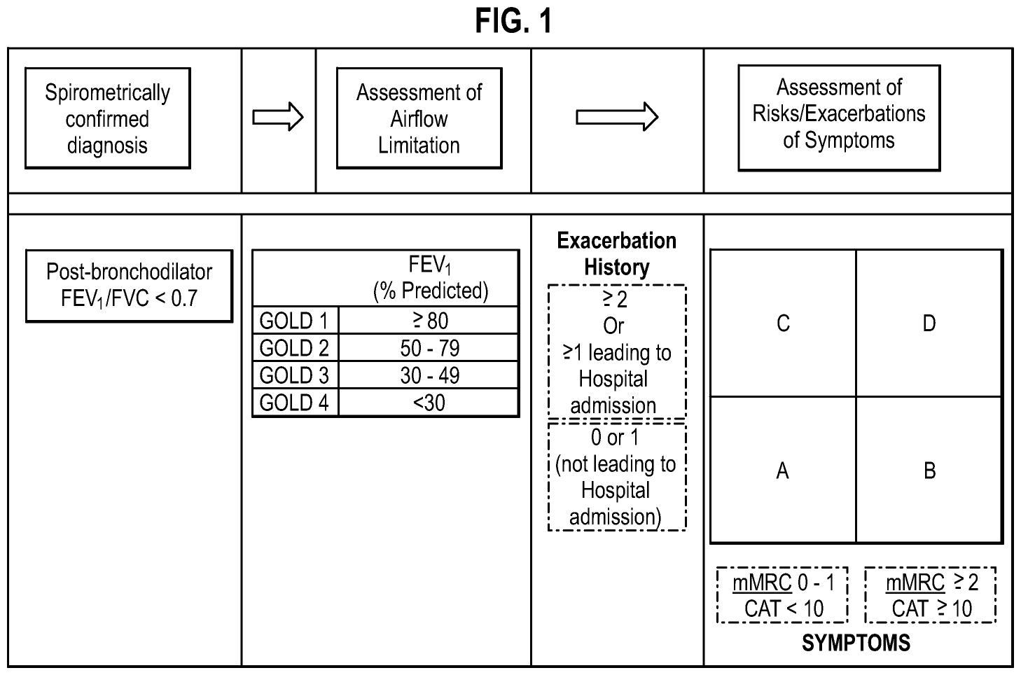

The “ABCD” assessment tool is further used to understand a COPD patient's severity of disease. This assessment combines the patient's spirometry analysis with their exacerbation history and symptom assessment to give a spirometric grade combined with an “ABCD” group. The ABCD assessment tool is shown in FIG. 1.

As described in the Examples (Table 10), a reduction in frequency of severe exacerbations has been observed in subjects having GOLD 4 (very severe) COPD status. Thus the immunogenic composition may be useful to reduce the frequency of severe exacerbations, in a subject (e.g. human) having GOLD 4 (very severe) COPD status. The immunogenic composition of the present invention may be useful in a subject (e.g. human) that has GOLD 4 (very severe) COPD status to reduce the frequency of severe exacerbations of COPD. Subjects having GOLD 4 (very severe) COPD status have an FEV₁<30% predicted. Thus, in an aspect there is provided an immunogenic composition for use, use of an immunogenic composition or method according to the present invention, wherein the subject has an FEV₁<30% predicted. There is also provided an immunogenic composition for use, use of an immunogenic composition or method according to the present invention, wherein the subject has GOLD 4 (very severe) COPD status. The immunogenic composition may be used to reduce the yearly rate of severe exacerbations wherein the subject has GOLD 4 (very severe) COPD status by at least 5%, for example by 10%, 20%, 30%, 40%, 50%, 60% or 65% in a subject that has been administered the immunogenic composition compared to a subject who has not been administered the immunogenic composition. In subjects having GOLD 4 (very severe) COPD status the yearly rate of severe exacerbations is reduced compared to a subject who has not been administered the immunogenic composition. Thus there is also provided an immunogenic composition for use, use of an immunogenic composition or method according to the present invention, wherein the subject has GOLD 4 (very severe) COPD status for reducing the yearly rate of severe exacerbations, e.g. by at least 19% (e.g. up to 75%), compared to a subject who has not been administered the immunogenic composition. There is also provided an immunogenic composition for use, use of an immunogenic composition or method according to the present invention, wherein the subject has GOLD 4 (very severe) COPD status for reducing the yearly rate of severe exacerbations, e.g. by 65% compared to a subject who has not been administered the immunogenic composition. The subject having GOLD 4 (very severe) COPD status may be selected and administered the immunogenic composition of the present invention. Thus the present invention also provides the immunogenic composition for use, use of an immunogenic composition or method for reducing the frequency of severe exacerbations, in a subject having COPD comprising: (a) selecting a subject who has GOLD 4 (very severe) COPD status, and (b) administering to the subject the immunogenic composition.

In one aspect, the present invention provides an immunogenic composition for use, use of an immunogenic composition or method according to the present invention, wherein the subject (e.g. a human aged 40 to 80 years old) has GOLD 4 (very severe) COPD status and has experienced at least 2 moderate episodes of acute exacerbation in chronic obstructive pulmonary disease (AECOPD) or at least one severe episode of AECOPD within a period of 12 months (e.g. in the previous 12 months). In another aspect, the present invention provides an immunogenic composition for use, use of an immunogenic composition or method according to the present invention, wherein the subject (e.g. a human aged 40 to 80 years old) has GOLD 4 (very severe) COPD status and has experienced at least one severe episode of AECOPD within a period of 12 months (e.g. in the previous 12 months).

### Particular Aspects of the Present Invention Include:

An immunogenic composition comprising: (i) an immunogenic polypeptide from non-typeable Haemophilus influenzae, (ii) a PE-PilA fusion protein, (iii) optionally an immunogenic polypeptide of UspA2 from Moraxella catarrhalis, and (iv) an adjuvant, for use in reducing the frequency of severe exacerbations, in a subject (e.g. human) having GOLD 4 (very severe) COPD status.
The use of an immunogenic composition comprising: (i) an immunogenic polypeptide from non-typeable Haemophilus influenzae, (ii) a PE-PilA fusion protein, (iii) optionally an immunogenic polypeptide of UspA2 from Moraxella catarrhalis, and (iv) an adjuvant, in the manufacture of a medicament for reducing the frequency of severe exacerbations, in a subject (e.g. human) having GOLD 4 (very severe) COPD status.
A method for reducing the frequency of severe exacerbations, in a subject (e.g. human) having GOLD 4 (very severe) COPD status, said method comprising administering to said subject a therapeutically effective amount of an immunogenic composition comprising: (i) an immunogenic polypeptide from non-typeable Haemophilus influenzae, (ii) a PE-PilA fusion protein, (iii) optionally an immunogenic polypeptide of UspA2 from Moraxella catarrhalis, and (iv) an adjuvant.
The immunogenic composition for use, use of an immunogenic composition or method as described above, for reducing the frequency of severe exacerbations in a subject (e.g. human) having GOLD 4 (very severe) COPD status, for reducing the yearly rate of severe exacerbations compared to a subject who has not been administered the immunogenic composition.
The immunogenic composition for use, use of an immunogenic composition or method as described above, for reducing the likelihood of hospitalization due to an AECOPD in a subject (e.g. human) having chronic obstructive pulmonary disease (COPD).
The immunogenic composition for use, use of an immunogenic composition or method as described above, for reducing the frequency of pneumonia due to COPD (e.g. due to an AECOPD) in a subject (e.g. human) having GOLD 4 (very severe) COPD status.
The immunogenic composition for use, use of an immunogenic composition or method as described above, for reducing the need for intensive care and/or mechanical ventilation due to COPD (e.g. due to an AECOPD) in a subject (e.g. human) having GOLD 4 (very severe) COPD status.
The immunogenic composition for use, use of an immunogenic composition or method as described above, for reducing the likelihood of mortality due to an AECOPD in a subject (e.g. human) having GOLD 4 (very severe) COPD status.
The immunogenic composition for use, use of an immunogenic composition or method as described above, in a subject (e.g. human) having GOLD 4 (very severe) COPD status, wherein the subject has an FEV₁<30% predicted.
The immunogenic composition for use, use of an immunogenic composition or method as described above, in a subject (e.g. human) having GOLD 4 (very severe) COPD status, for reducing the yearly rate of severe exacerbations, e.g. by at least 19%, compared to a subject who has not been administered the immunogenic composition.
The immunogenic composition for use, use of an immunogenic composition or method as described above for reducing the frequency of severe exacerbations, in a subject having COPD comprising: (a) selecting a subject who has GOLD 4 (very severe) COPD status, and (b) administering to the subject the immunogenic composition.
The immunogenic composition for use, use of an immunogenic composition or method as described above, in a subject (e.g. human) having GOLD 4 (very severe) COPD status, wherein the subject has experienced at least 2 moderate AECOPD or at least one severe AECOPD in the previous 12 months.
The immunogenic composition for use, use of an immunogenic composition or method as described above, in a subject (e.g. human) having GOLD 4 (very severe) COPD status, wherein the subject has experienced at least 2 moderate AECOPD or at least one severe AECOPD in the previous 12 months and is taking ICS.
The immunogenic composition for use, use of an immunogenic composition or method as described above, in a subject (e.g. human) having GOLD 4 (very severe) COPD status, wherein the subject has experienced at least 2 moderate AECOPD or at least one severe AECOPD in the previous 12 months and is taking short term and long term bronchodilators and ICS.
The immunogenic composition for use, use of an immunogenic composition or method as described above, in a subject (e.g. human) having GOLD 4 (very severe) COPD status, wherein the subject has experienced at least 2 moderate AECOPD or at least one severe AECOPD in the previous 12 months for reducing the yearly rate of severe exacerbations compared to a subject who is not administered the immunogenic composition.
The immunogenic composition for use, use of an immunogenic composition or method as described above for reducing the frequency of severe exacerbations, in a subject (e.g. human) having chronic obstructive pulmonary disease (COPD) comprising: (a) selecting a subject who has GOLD 4 (very severe) COPD status that is partially controlled or uncontrolled by ICS and has experienced at least 2 moderate AECOPD or at least one severe AECOPD in the previous 12 months, and (b) administering to the subject the immunogenic composition.
The immunogenic composition for use, use of an immunogenic composition or method as described above in a subject (e.g. human) having GOLD 4 (very severe) COPD status wherein the subject has previously been administered a pneumococcal vaccine.
The immunogenic composition for use, use of an immunogenic composition or method as described above in a subject (e.g. human) having GOLD 4 (very severe) COPD status wherein the subject has been administered an influenza vaccine in previous 12 months.
The immunogenic composition for use, use of an immunogenic composition or method as described above in a subject (e.g. human) having GOLD 4 (very severe) COPD status wherein the subject has previously been administered a pneumococcal vaccine and/or has been administered an influenza vaccine in the previous 12 months.
The immunogenic composition for use, use of an immunogenic composition or method as described above in a subject (e.g. human) having GOLD 4 (very severe) COPD status wherein the subject has previously been administered a pneumococcal vaccine for reducing the yearly rate of severe exacerbations compared to a subject who has not been administered the immunogenic composition.
The immunogenic composition for use, use of an immunogenic composition or method as described above in a subject (e.g. human) having GOLD 4 (very severe) COPD status for reducing the rate of severe exacerbations (and related hospitalizations) during the winter season (December-February in Europe).
The immunogenic composition for use, use of an immunogenic composition or method as described above in a subject (e.g. human) having GOLD 4 (very severe) COPD status for reducing the rate of severe exacerbations (and related hospitalizations) during the spring season (March-May in Europe).
The immunogenic composition for use, use of an immunogenic composition or method as described above in a subject (e.g. human) having GOLD 4 (very severe) COPD status for reducing the rate of severe exacerbations (and related hospitalizations) during the summer season (June-August in Europe).
The immunogenic composition for use, use of an immunogenic composition or method as described above in a subject (e.g. human) having GOLD 4 (very severe) COPD status for reducing the rate of severe exacerbations (and related hospitalizations) during the autumn season (September-November in Europe).
The immunogenic composition for use, use of an immunogenic composition or method as described above in a subject (e.g. human) having GOLD 4 (very severe) COPD status, wherein the immunogenic composition comprises an immunogenic polypeptide of Protein D, suitably an isolated immunogenic polypeptide with at least 70%, 80%, 85%, 90%, 91%, 92%, 93%, 94%, 95%, 96%, 97%, 98%, 99%, or 100% to SEQ ID NO: 2.
The immunogenic composition for use, use of an immunogenic composition or method as described above in a subject (e.g. human) having GOLD 4 (very severe) COPD status, wherein the immunogenic composition comprises a PE-PilA fusion protein, suitably an isolated immunogenic polypeptide with at least 70%, 80%, 85%, 90%, 91%, 92%, 93%, 94%, 95%, 96%, 97%, 98%, 99%, or 100% to SEQ ID NO: 9.
The immunogenic composition for use, use of an immunogenic composition or method as described above in a subject (e.g. human) having GOLD 4 (very severe) COPD status, wherein the composition comprises an immunogenic polypeptide of UspA2 from Moraxella catarrhalis, suitably an isolated immunogenic polypeptide with at least 70%, 80%, 85%, 90%, 91%, 92%, 93%, 94%, 95%, 96%, 97%, 98%, 99%, or 100% to a polypeptide selected from the group consisting of MC-001 (SEQ ID NO: 11), MC-002 (SEQ ID NO: 12), MC-003 (SEQ ID NO: 13), MC-004 (SEQ ID NO: 14), MC-005 (SEQ ID NO: 15), MC-006 (SEQ ID NO: 16), MC-007 (SEQ ID NO: 17), MC-008 (SEQ ID NO:18), MC-009 (SEQ ID NO: 19), MC-010 (SEQ ID NO: 20) or MC-011 (SEQ ID NO: 21) e.g. MC009 SEQ ID NO: 19.
The immunogenic composition for use, use of an immunogenic composition or method as described above in a subject (e.g. human) having GOLD 4 (very severe) COPD status, wherein the immunogenic composition comprises a pharmaceutically acceptable excipient or carrier.
The immunogenic composition for use, use of an immunogenic composition or method as described above in a subject (e.g. human) having GOLD 4 (very severe) COPD status, wherein the adjuvant is an AS01 adjuvant, e.g. ASO1E.

### Pharmacologic Therapy

A number of medications are available for the treatment of COPD and these are used mainly to reduce symptoms and reduce the frequency and severity of exacerbations and can include bronchodilators, such as beta₂-agonists and anticholinergics, corticosteroids (oral and inhaled), PDE-4 inhibitors, methylxanthines and combinations thereof. Bronchodilator medications are vital to symptom management in COPD and the choice between monotherapy with a beta₂-agonist, anticholinergic or theophylline, or combination therapy is dependent upon how effective the medication controls a patient's symptoms.

According to the 2014 Global Initiative for Chronic Obstructive Lung Disease (GOLD) strategy, for COPD patients with a high risk of exacerbations, inhaled corticosteroids are recommended in combination with a beta2-agonist or anticholinergic. However, exacerbations of COPD remain a major risk for patients suffering with COPD is a major cause of COPD related hospitalizations and mortality. Improved treatment options are sought.

Pharmacologic therapy for COPD is utilised to control and reduce symptoms, reduce the frequency and severity of exacerbations and improve tolerance to exercise. Classes of therapeutic agents that can be used to treat COPD include, but are not limited to, beta₂-agonists, anticholinergics, methylxanthines and phosphodiesterase-4 (PDE-4) inhibitors. Anti-inflammatory agents, such as inhaled corticosteroids are also used, typically in combination with a beta₂-agonist and/or an antibcholinergic.

Beta₂agonists include, but are not limited to, short-acting beta₂-agonists such as fenoterol, levalbuterol, salbutamol, terbutaline, and long-acting beta₂-agonists such as arformoterol, formoterol, indacaterol, vilanterol (e.g. vilanterol as the acetate, 1-naphthoate, (R)-mandelate, α-phenylcinnamate or triphenylacetate (trifenatate) salt), olodaterol and salmeterol (e.g. salmeterol xinafoate).

Anticholinergics include, but are not limited to, short-acting anticholinergics such as ipratropium (e.g. ipratropium bromide), oxitropium (e.g. oxitropium bromide), and long-acting anticholinergics such as aclidinium (e.g. aclidinium bromide, glycopyrronium (e.g. glycopyrronium bromide), tiotropium (e.g. tiotropium bromide) and umeclidinium (e.g. umeclidinium bromide).

Methylxanthines include, but are not limited to, aminophylline and theophylline (SR).

It will be clear to the person skilled in the art that, where appropriate, the other therapeutic agents may be used in the form of salts, prodrugs, esters, or solvates (e.g. hydrates) to optimise the activity and/or stability and/or physical characteristics (e.g. solubility) of the therapeutic agent. The additional therapeutic agents may be used in optically pure form and in either amorphous or crystalline form.

The present invention further provides an immunogenic composition for use, use of an immunogenic composition or method according to the present invention, wherein the subject is taking one or more other therapeutic agents for COPD. The present invention also provides an immunogenic composition, use or method according to the present invention, wherein the subject has been prescribed one or more other therapeutic agents for COPD. In an embodiment, the present invention provides an immunogenic composition for use, use of an immunogenic composition or method according to the present invention, wherein the subject is taking an inhaled corticosteroid (ICS), for example fluticasone furoate.

Combinations of pharmacologic therapies may also be used for the treatment of COPD. In an embodiment, the therapy is a dual combination of a beta₂-agonist and an inhaled corticosteroid, such as the combination of vilanterol, or a pharmaceutically acceptable salt or solvate thereof, and fluticasone furoate. In one embodiment, the therapy is a combination of vilanterol trifenatate and fluticasone furoate.

Pharmacologic therapy may also include the combination of three classes of therapeutic agents, such as the combination of a beta₂-agonist, an anticholinergic and an inhaled corticosteroid. In an embodiment, the therapy is a combination of vilanterol, or a pharmaceutically acceptable salt or solvate thereof, umeclidinium, or a pharmaceutically acceptable salt or solvate thereof, and fluticasone furoate. In a further embodiment, the therapy is a combination of vilanterol trifenatate, umeclidinium bromide and fluticasone furoate.

In an embodiment, the present invention provides an immunogenic composition for use, use of an immunogenic composition or method according to the present invention, wherein the subject is taking one or more other therapeutic agents for COPD selected from the group consisting of beta₂-agonists, anticholinergics, methylxanthines, phosphodiesterase-4 (PDE-4) inhibitors and inhaled corticosteroids.

In an embodiment, the present invention provides an immunogenic composition for use, use of an immunogenic composition or method according to the present invention, wherein the subject is taking a combination of a beta₂-agonist, for example vilanterol (e.g. vilanterol trifenatate), and an anticholinergic, for example umeclidinium (e.g. umeclidinium bromide), for COPD.

In an embodiment, the present invention provides an immunogenic composition for use, use of an immunogenic composition or method according to the present invention, wherein the subject is taking a combination of a beta₂-agonist, for example vilanterol (e.g. vilanterol trifenatate), and an inhaled corticosteroid, for example fluticasone furoate, for COPD.

In an embodiment, the present invention provides an immunogenic composition for use, use of an immunogenic composition or method according to the present invention, wherein the subject is taking a combination of a beta₂-agonist, for example vilanterol (e.g. vilanterol trifenatate), an anticholinergic, for example umeclidinium (e.g. umeclidinium bromide), and an inhaled corticosteroid, for example fluticasone furoate, for COPD.

Pharmacologic therapy may be formulated as solutions, suspensions or as dry powder compositions typically for inhalation via a reservoir dry powder inhaler, unit-dose dry powder inhaler, per-metered multi-dose dry powder inhaler, nasal inhaler, pressurised metered dose inhaler, or nebuliser. Representative dry powder inhalers are the DISKHALER™ inhaler device, the DISKUS™ inhalation device, and the ELLIPTA™ inhalation device, marketed by GlaxoSmithKline. The DISKUS™ inhalation device is, for example, described in GB 2242134A, and the ELLIPTA™ inhalation device is, for example, described in WO 2003/061743 A1, WO 2007/012871 A1 and/or WO 2007/068896 A1.

Dry powder compositions may be presented in unit dose form, as capsules, cartridges or commonly blisters. Umeclidinium, for example umeclidinium bromide, may be formulated as a dry powder composition, wherein umeclidinium is to be administered at a dose of 62.5 mcg or 125 mcg once daily, wherein the dose is the amount of the free cation (i.e. umeclidinium).

Vilanterol, for example vilanterol trifenatate, may be formulated as a dry powder composition, wherein vilanterol is to be administered at a dose of 25 mcg once daily, wherein the dose is the amount of the free base (i.e. vilanterol).

Fluticasone furoate may be formulated as a dry powder composition, wherein fluticasone furoate is to be administered at a dose of 50 mcg, 100 mcg, or 200 mcg once daily. In an embodiment, the dose is 100 mcg once daily.

Individual therapeutic agents may be administered sequentially or simultaneously in separate or combined pharmaceutical formulations/compositions. Where appropriate, the individual therapeutic agents may be admixed within the same formulation, and presented as a fixed pharmaceutical combination. In general such formulations/compositions will include pharmaceutical carriers or excipients.

In an embodiment, the present invention provides an immunogenic composition for use, use of an immunogenic composition or method according to the present invention, wherein the subject is taking a combination of a beta₂-agonist, for example vilanterol trifenatate at a dose of 25 mcg once daily, and an inhaled corticosteroid, for example fluticasone furoate at a dose of 100 mcg once daily, for COPD. In a further embodiment, the subject is taking the product Breo™.

In an embodiment, the present invention provides an immunogenic composition for use, use of an immunogenic composition or method according to the present invention, wherein the subject is taking a combination of vilanterol trifenatate and fluticasone furoate for COPD, wherein vilanterol trifenatate and fluticasone furoate are contained within the same dry powder inhaler device (e.g. the Ellipta™ Inhaler), wherein the dry powder inhaler device contains two blister strips, wherein one strip contains a blend of micronised fluticasone furoate (approximately 100 mcg per blister) and lactose monohydrate, and a second strip contains vilanterol trifenatate (approximately 25 mcg per blister of vilanterol), lactose monohydrate and magnesium stearate (for example at about 1.0% w/w based on the total weight of the dry powder composition).

In an embodiment, the present invention provides an immunogenic composition for use, use of an immunogenic composition or method according to the present invention, wherein the subject is taking a combination of a beta₂-agonist, for example vilanterol trifenatate at a dose of 25 mcg once daily, an anticholinergic, for example umeclidinium bromide at a dose of 62.5 mcg once daily, and an inhaled corticosteroid, for example fluticasone furoate at a dose of 100 mcg once daily, for COPD.

In an embodiment, the present invention provides an immunogenic composition for use, use of an immunogenic composition or method according to the present invention, wherein the subject is taking a combination of a vilanterol trifenatate, umeclidinium bromide and fluticasone furoate for COPD, wherein umeclidinium bromide, vilanterol trifenatate and fluticasone furoate are contained within the same dry powder inhaler device (e.g. the Ellipta™ Inhaler), wherein the dry powder inhaler device contains two blister strips, wherein one strip contains a blend of micronised umeclidinium bromide (approximately 125 or 62.5 mcg per blister of umeclidinium), vilanterol trifenatate (approximately 25 mcg per blister of vilanterol), magnesium stearate (for example at about 1.0% w/w/based on the total weight of the dry powder composition) and lactose monohydrate, and a second strip contains a blend of micronized fluticasone furoate (approximately 100 mcg per blister) and lactose monohydrate.

The inhaler device may deliver, when actuated, the contents of a single blister simultaneously from each of the two blister strips. Each blister strip may be a double foil laminate containing 7, 14 or 30 filled blisters per strip.

All COPD patients, in particular those with moderate to very severe persistant airflow obstruction classified as either GOLD 2, GOLD 3 or GOLD 4, are prone to exacerbations and thus may benefit from treatment or prevention with an immunogenic composition of the present invention when added to existing maintenance therapy for COPD. In an embodiment, an immunogenic composition of the present invention may be used in the treatment of human subjects (e.g. patients) with moderate to very severe COPD with a previous history of acute exacerbations (AECOPD), for example at least one (e.g. 2 or more, 3 or more) episodes of moderate to very severe AECOPD within the previous 12 months. In another embodiment, combination of an immunogenic composition of the present invention with existing ICS maintenance therapy may be used in the treatment of human subjects (e.g. patients) with moderate to very severe COPD with a previous history of acute exacerbations (AECOPD), for example at least one (e.g. 2 or more, 3 or more) episodes of moderate to very severe AECOPD within the previous 12 months.

The addition of an immunogenic composition according to the present invention to a patient's existing maintenance therapy may be complementary, and, for example, may reduce the frequency of severe exacerbations of COPD.

### Use/Methods of Treatment in Frequest Exacerbators Taking ICS

A subject suffering from COPD may be taking inhaled corticosteroids (ICS) as a maintenance therapy. Exacerbations of COPD may be treated with systemic corticosteroids and/or antibiotics. If a subject is then identified as suffering from an exacerbation of COPD that is associated with a bacterial infection, an antibiotic agent may be preferred to, or used in combination with, a corticosteroid based therapy.

A maintenance therapy is a therapy which is administered subsequent to an induction therapy (an initial course of therapy administered to an individual or subject with COPD). Maintenance therapy can be used to halt or reverse the progression of the disease/disorder. Maintenance therapy may be administered as a baseline therapy in contrast to therapies which are administered only in response to exacerbations. Maintenance therapy may be prescribed long term, e.g. for more than one month, for more than one year etc.

The immunogenic composition of the present invention may also be useful in a subject (e.g. human) that is taking ICS as maintenance therapy to reduce the frequency of severe exacerbations of COPD. As described in the Examples (Table 9(a)), a reduction in frequency of severe exacerbations was observed in subjects that are “frequent exacerbators” having experienced at least 2 moderate AECOPD or at least one severe AECOPD in the previous 12 months that were taking ICS. The immunogenic composition of the present invention may also be useful in subject that has experienced at least 2 moderate AECOPD or at least one severe AECOPD in the previous 12 months and is taking ICS as a maintenance therapy to reduce the frequency of severe exacerbations of COPD. The subject may continue their use of a maintenance inhaled corticosteroid and be administered the immunogenic composition in addition. Thus, in a further aspect of the present invention there is provided the immunogenic composition for use, use of an immunogenic composition or method according to the invention, wherein the subject has experienced at least 2 moderate AECOPD or at least one severe AECOPD in the previous 12 months and is taking ICS. There is also provided the immunogenic composition for use, use of an immunogenic composition or method according to the present invention, wherein the subject has experienced at at least one severe AECOPD in the previous 12 months and is taking ICS. Examples of inhaled corticosteroids (ICS) include beclomethasone dipropionate, budesonide, ciclesonide, flunisolide, fluticasone propionate, fluticasone furoate, or mometasone furoate. The ICS may for example be fluticasone fuorate.

The present invention also provides the immunogenic composition for use, use of an immunogenic composition or method according to the present invention, wherein the subject has experienced at least 2 moderate AECOPD or at least one severe AECOPD in the previous 12 months and is taking short term and long term bronchodilators and ICS. Thus, in addition to ICS the patient may be taking one or more therapeutic agents selected from the group consisting of beta₂-agonists, anticholinergics, methylxanthines, phosphodiesterase-4 (PDE-4) inhibitors.

The present invention may also be useful where the subject has experienced an acute exacerbation of chronic obstructive pulmonary disease (AECOPD) and has failed to achieve resolution of symptoms after ICS therapy. Thus, the present invention also provides the immunogenic composition for use, use of an immunogenic composition or method according to the invention for reducing the frequency of severe exacerbations, in a subject having COPD comprising: (a) selecting a subject who has COPD that is partially controlled or uncontrolled by ICS and has experienced at least 2 moderate AECOPD or at least one severe AECOPD in the previous 12 months, and (b) administering to the subject the immunogenic composition.

There is also provided an immunogenic composition for use, use of an immunogenic composition or method according to the present invention, wherein the subject has experienced at least 2 moderate AECOPD or at least one severe AECOPD in the previous 12 months and is taking ICS. The immunogenic compositions may be used to reduce the yearly rate of severe exacerbations wherein the subject has experienced at least 2 moderate AECOPD or at least one severe AECOPD in the previous 12 months and is taking ICS, by at least 5%, for example by 10%, 20%, 30%, 40%, 50%, 60%, 70% or 75%, in a subject that has been administered the immunogenic composition compared to a subject who has not been administered the immunogenic composition. In subjects which have experienced at least 2 moderate AECOPD or at least one severe AECOPD in the previous 12 months and are taking ICS, the yearly rate of severe exacerbations is reduced, e.g. by 75%, compared to a subject who has not been administered the immunogenic composition. Thus there is also provided an immunogenic composition for use, use of an immunogenic composition or method according to the present invention, wherein the subject has experienced at least 2 moderate AECOPD or at least one severe AECOPD in the previous 12 months and is taking ICS for reducing the yearly rate of severe exacerbations, e.g. by 75%, compared to a subject who has not been administered the immunogenic composition. The subject that has experienced at least 2 moderate AECOPD or at least one severe AECOPD in the previous 12 months and is taking ICS may be selected and administered the immunogenic composition of the present invention. Thus the present invention also provides the immunogenic composition for use, use of an immunogenic composition or method for reducing the frequency of severe exacerbations, in a subject having COPD comprising: (a) selecting a subject who has experienced at least 2 moderate AECOPD or at least one severe AECOPD in the previous 12 months and is taking ICS, and (b) administering to the subject the immunogenic composition.

As described in the Examples (Table 9(b)), a reduction in frequency of severe exacerbations was observed in subjects that were taking ICS. The immunogenic composition of the present invention may also be useful in subject that is taking ICS as a maintenance therapy to reduce the frequency of severe exacerbations of COPD. The subject may continue their use of a maintenance inhaled corticosteroid and be administered the immunogenic composition in addition. Thus, in a further aspect of the present invention there is provided the immunogenic composition for use, use of an immunogenic composition or method according to the invention, wherein the subject is taking ICS. There is also provided the immunogenic composition for use, use of an immunogenic composition or method according to the present invention, wherein the subject is taking ICS. Examples of inhaled corticosteroids (ICS) include beclomethasone dipropionate, budesonide, ciclesonide, flunisolide, fluticasone propionate, fluticasone furoate, or mometasone furoate. The ICS may for example be fluticasone fuorate.

The present invention also provides the immunogenic composition for use, use of an immunogenic composition or method according to the present invention, wherein the subject is taking short term and long term bronchodilators and ICS. Thus, in addition to ICS the patient may be taking one or more therapeutic agents selected from the group consisting of beta₂-agonists, anticholinergics, methylxanthines, phosphodiesterase-4 (PDE-4) inhibitors.

The present invention may also be useful where the subject has experienced an acute exacerbation of chronic obstructive pulmonary disease (AECOPD) and has failed to achieve resolution of symptoms after ICS therapy. Thus, the present invention also provides the immunogenic composition for use, use of an immunogenic composition or method according to the invention for reducing the frequency of severe exacerbations, in a subject having COPD comprising: (a) selecting a subject who has COPD that is partially controlled or uncontrolled by ICS, and (b) administering to the subject the immunogenic composition.

There is also provided an immunogenic composition for use, use of an immunogenic composition or method according to the present invention, wherein the subject is taking ICS. The immunogenic compositions may be used to reduce the yearly rate of severe exacerbations wherein the subject is taking ICS, by at least 5%, for example by 10%, 20%, 30%, 40%, 50%, 60% or 61%, in a subject that has been administered the immunogenic composition compared to a subject who has not been administered the immunogenic composition. In subjects which are taking ICS, the yearly rate of severe exacerbations is reduced, e.g. by 61%, compared to a subject who has not been administered the immunogenic composition. Thus there is also provided an immunogenic composition for use, use of an immunogenic composition or method according to the present invention, wherein the subject is taking ICS for reducing the yearly rate of severe exacerbations, e.g. by 61%, compared to a subject who has not been administered the immunogenic composition. The subject that is taking ICS may be selected and administered the immunogenic composition of the present invention. Thus the present invention also provides the immunogenic composition for use, use of an immunogenic composition or method for reducing the frequency of severe exacerbations, in a subject having COPD comprising: (a) selecting a subject that is taking ICS, and (b) administering to the subject the immunogenic composition.

Particular Aspects of the Present Invention Include:
An immunogenic composition comprising: (i) an immunogenic polypeptide from non-typeable Haemophilus influenzae, (ii) a PE-PilA fusion protein, (iii) optionally an immunogenic UspA2 polypeptide from Moraxella catarrhalis, and (iv) an adjuvant, for use in reducing the frequency of severe exacerbations, in a subject (e.g. human) having chronic obstructive pulmonary disease (COPD) wherein the subject is taking ICS.
The use of an immunogenic composition comprising: (i) an immunogenic polypeptide from non-typeable Haemophilus influenzae, (ii) a PE-PilA fusion protein, (iii) optionally an immunogenic UspA2 polypeptide from Moraxella catarrhalis, and (iv) an adjuvant, in the manufacture of a medicament for reducing the frequency of severe exacerbations, in a subject (e.g. human) having chronic obstructive pulmonary disease (COPD) wherein the subject is taking ICS.
A method for reducing the frequency of severe exacerbations, in a subject (e.g. human) having chronic obstructive pulmonary disease (COPD), said method comprising administering to said subject a therapeutically effective amount of an immunogenic composition comprising: (i) an immunogenic polypeptide from non-typeable Haemophilus influenzae, (ii) a PE-PilA fusion protein, and (iii) optionally an immunogenic UspA2 polypeptide from Moraxella catarrhalis, and (iv) an adjuvant, wherein the subject is taking ICS.
The immunogenic composition for use, use of an immunogenic composition or method as described above, in a subject (e.g. human) having chronic obstructive pulmonary disease (COPD) wherein the subject is taking ICS and the subject has experienced at least 2 moderate AECOPD or at least one severe AECOPD in the previous 12 months.
The immunogenic composition for use, use of an immunogenic composition or method as described above, in a subject (e.g. human) having chronic obstructive pulmonary disease (COPD) wherein the subject is taking ICS (and optionally the subject has experienced at least 2 moderate AECOPD or at least one severe AECOPD in the previous 12 months), for reducing the yearly rate of severe exacerbations compared to a subject who has not been administered the immunogenic composition.
The immunogenic composition for use, use of an immunogenic composition or method as described above, for reducing the frequency of pneumonia due to COPD (e.g. due to an AECOPD) in a subject (e.g. human) having chronic obstructive pulmonary disease (COPD) wherein the subject is taking ICS (and optionally the subject has experienced at least 2 moderate AECOPD or at least one severe AECOPD in the previous 12 months).
The immunogenic composition for use, use of an immunogenic composition or method as described above, for reducing the need for intensive care and/or mechanical ventilation due to COPD (e.g. due to an AECOPD) in a subject (e.g. human) having chronic obstructive pulmonary disease (COPD) wherein the subject is taking ICS (and optionally the subject has experienced at least 2 moderate AECOPD or at least one severe AECOPD in the previous 12 months).
The immunogenic composition for use, use of an immunogenic composition or method as described above, for reducing the likelihood of mortality due to an AECOPD in a subject (e.g. human) having chronic obstructive pulmonary disease (COPD) wherein the subject is taking ICS (and optionally the subject has experienced at least 2 moderate AECOPD or at least one severe AECOPD in the previous 12 months).
The immunogenic composition for use, use of an immunogenic composition or method as described above, in a subject (e.g. human) having chronic obstructive pulmonary disease (COPD) wherein is taking ICS (and optionally the subject has experienced at least 2 moderate AECOPD or at least one severe AECOPD in the previous 12 months), and wherein the subject has an FEV₁<30% predicted.
The immunogenic composition for use, use of an immunogenic composition or method as described above, in a subject (e.g. human) having chronic obstructive pulmonary disease (COPD) wherein the subject is taking ICS (and optionally the subject has experienced at least 2 moderate AECOPD or at least one severe AECOPD in the previous 12 months), and wherein the subject has GOLD 4 (very severe) COPD status.
The immunogenic composition for use, use of an immunogenic composition or method as described above, in a subject (e.g. human) having chronic obstructive pulmonary disease (COPD) wherein the subject is taking ICS (and optionally the subject has experienced at least 2 moderate AECOPD or at least one severe AECOPD in the previous 12 months), and wherein the subject has GOLD 4 (very severe) COPD status, for reducing the yearly rate of severe exacerbations compared to a subject who has not been administered the immunogenic composition.
The immunogenic composition for use, use of an immunogenic composition or method as described above for reducing the frequency of severe exacerbations, in a subject (e.g. human) having chronic obstructive pulmonary disease (COPD) comprising: (a) selecting a subject who has GOLD 4 (very severe) COPD status, is taking ICS (and optionally the subject has experienced at least 2 moderate AECOPD or at least one severe AECOPD in the previous 12 months), and (b) administering to the subject the immunogenic composition.
The immunogenic composition for use, use of an immunogenic composition or method as described above, in a subject (e.g. human) having chronic obstructive pulmonary disease (COPD) wherein the subject is taking short term and long term bronchodilators and ICS (and optionally the subject has experienced at least 2 moderate AECOPD or at least one severe AECOPD in the previous 12 months).
The immunogenic composition for use, use of an immunogenic composition or method as described above, in a subject (e.g. human) having chronic obstructive pulmonary disease (COPD) wherein the subject is taking ICS (and optionally the subject has experienced at least 2 moderate AECOPD or at least one severe AECOPD in the previous 12 months) for reducing the yearly rate of severe exacerbations compared to a subject who is not administered the immunogenic composition.
The immunogenic composition for use, use of an immunogenic composition or method as described above for reducing the frequency of severe exacerbations, in a subject having COPD comprising: (a) selecting a subject who has COPD that is partially controlled or uncontrolled by ICS (and optionally the subject has experienced at least 2 moderate AECOPD or at least one severe AECOPD in the previous 12 months), and (b) administering to the subject the immunogenic composition.
The immunogenic composition for use, use of an immunogenic composition or method as described above in a subject (e.g. human) having chronic obstructive pulmonary disease (COPD) wherein the subject is taking ICS (and optionally the subject has experienced at least 2 moderate AECOPD or at least one severe AECOPD in the previous 12 months), for reducing the rate of severe exacerbations (and related hospitalizations) during the winter season (December-February in Europe).
The immunogenic composition for use, use of an immunogenic composition or method as described above in a subject (e.g. human) having chronic obstructive pulmonary disease (COPD) wherein the subject is taking ICS (and optionally the subject has experienced at least 2 moderate AECOPD or at least one severe AECOPD in the previous 12 months), for reducing the rate of severe exacerbations (and related hospitalizations) during the spring season (March-May in Europe).
The immunogenic composition for use, use of an immunogenic composition or method as described above in a subject (e.g. human) having chronic obstructive pulmonary disease (COPD) wherein the subject is taking ICS (and optionally the subject has experienced at least 2 moderate AECOPD or at least one severe AECOPD in the previous 12 months), for reducing the rate of severe exacerbations (and related hospitalizations) during the summer season (June-August in Europe).
The immunogenic composition for use, use of an immunogenic composition or method as described above in a subject (e.g. human) having chronic obstructive pulmonary disease (COPD) wherein the subject is taking ICS (and optionally the subject has experienced at least 2 moderate AECOPD or at least one severe AECOPD in the previous 12 months), for reducing the rate of severe exacerbations (and related hospitalizations) during the autumn season (September-November in Europe).
The immunogenic composition for use, use of an immunogenic composition or method as described above, in a subject (e.g. human) having chronic obstructive pulmonary disease (COPD) wherein subject is taking ICS (and optionally the subject has experienced at least 2 moderate AECOPD or at least one severe AECOPD in the previous 12 months), and wherein the immunogenic composition comprises an immunogenic polypeptide of Protein D, suitably an isolated immunogenic polypeptide with at least 70%, 80%, 85%, 90%, 91%, 92%, 93%, 94%, 95%, 96%, 97%, 98%, 99%, or 100% to SEQ ID NO: 2.
The immunogenic composition for use, use of an immunogenic composition or method as described above, in a subject (e.g. human) having chronic obstructive pulmonary disease (COPD) wherein the subject is taking ICS (and optionally the subject has experienced at least 2 moderate AECOPD or at least one severe AECOPD in the previous 12 months), and wherein the immunogenic composition comprises a PE-PilA fusion protein, suitably an isolated immunogenic polypeptide with at least 70%, 80%, 85%, 90%, 91%, 92%, 93%, 94%, 95%, 96%, 97%, 98%, 99%, or 100% to SEQ ID NO: 9.
The immunogenic composition for use, use of an immunogenic composition or method as described above, in a subject (e.g. human) having chronic obstructive pulmonary disease (COPD) wherein the subject is taking ICS (and optionally the subject has experienced at least 2 moderate AECOPD or at least one severe AECOPD in the previous 12 months), and wherein the composition comprises an immunogenic polypeptide of UspA2 from Moraxella catarrhalis, suitably an isolated immunogenic polypeptide with at least 70%, 80%, 85%, 90%, 91%, 92%, 93%, 94%, 95%, 96%, 97%, 98%, 99%, or 100% to a polypeptide selected from the group consisting of MC-001 (SEQ ID NO: 11), MC-002 (SEQ ID NO: 12), MC-003 (SEQ ID NO: 13), MC-004 (SEQ ID NO: 14), MC-005 (SEQ ID NO: 15), MC-006 (SEQ ID NO: 16), MC-007 (SEQ ID NO: 17), MC-008 (SEQ ID NO:18), MC-009 (SEQ ID NO: 19), MC-010 (SEQ ID NO: 20) or MC-011 (SEQ ID NO: 21) e.g. MC009 SEQ ID NO: 19.
The immunogenic composition for use, use of an immunogenic composition or method as described above, in a subject (e.g. human) having chronic obstructive pulmonary disease (COPD) wherein the subject is taking ICS (and optionally the subject has experienced at least 2 moderate AECOPD or at least one severe AECOPD in the previous 12 months), and wherein the immunogenic composition comprises a pharmaceutically acceptable excipient or carrier.
The immunogenic composition for use, use of an immunogenic composition or method as described above, in a subject (e.g. human) having chronic obstructive pulmonary disease (COPD) wherein the subject is taking ICS (and optionally the subject has experienced at least 2 moderate AECOPD or at least one severe AECOPD in the previous 12 months), and wherein the adjuvant is an AS01 adjuvant, e.g. AS01E.
   Use/Methods of Treatment in Subjects who have been administered Pneumococcal and/or Influenza Vaccines

As described in the Examples (Table 11 and Table 12), a reduction in frequency of severe exacerbations was observed in subjects that had been administered pneumococcal and/or influenza vaccines. Thus the immunogenic composition may be useful to reduce the frequency of severe exacerbations in a subject (e.g. human) that have been administered pneumococcal and/or influenza vaccines. The immunogenic composition of the present invention may be useful in a subject (e.g. human) that has been administered pneumococcal and/or influenza vaccines to reduce the frequency of severe exacerbations of COPD.

Thus the present invention provides the immunogenic composition for use, use of an immunogenic composition or method according to the present invention, wherein the subject has previously been administered a pneumococcal vaccine. The present invention also provides the immunogenic composition for use, use of an immunogenic composition or method according to the present invention wherein the subject has been administered an influenza vaccine in previous 12 months. The present invention also provides the immunogenic composition for use, use of an immunogenic composition or method according to the present invention wherein the subject has previously been administered a pneumococcal vaccine and/or has been administered an influenza vaccine in the previous 12 months. The present invention also provides the immunogenic composition for use, use of an immunogenic composition or method according to the present invention wherein the subject has previously been administered a pneumococcal vaccine for reducing the yearly rate of severe exacerbations, e.g. by at least 2% (e.g. up to 77%), compared to a subject who has not been administered the immunogenic composition. The immunogenic compositions may be used to reduce the yearly rate of severe exacerbations wherein the subject has previously been administered a pneumococcal vaccine, by at least 2%, for example by 10%, 20%, 30%, 35%, 40%, 45%, 50% or 53%, in a subject that has been administered the immunogenic composition compared to a subject who has not been administered the immunogenic composition. The immunogenic compositions may be used to reduce the yearly rate of severe exacerbations wherein the subject has previously been administered a pneumococcal vaccine, e.g. by 53%, in a subject that has been administered the immunogenic composition compared to a subject who has not been administered the immunogenic composition.

The subject having previously been administered a pneumococcal vaccine and/or has been administered an influenza vaccine in the previous 12 months may be selected and administered the immunogenic composition of the present invention. Thus the present invention also provides the immunogenic composition for use, use of an immunogenic composition or method for reducing the frequency of severe exacerbations, in a subject having COPD comprising: (a) selecting a subject who has previously been administered a pneumococcal vaccine and/or has been administered an influenza vaccine in the previous 12 months, and (b) administering to the subject the immunogenic composition.

In one aspect, the present invention provides an immunogenic composition for use, use of an immunogenic composition or method according to the present invention, wherein the subject (e.g. a human aged 40 to 80 years old) has previously been administered a pneumococcal vaccine and/or has been administered an influenza vaccine in the previous 12 months and has experienced at least 2 moderate episodes of acute exacerbation in chronic obstructive pulmonary disease (AECOPD) or at least one severe episode of AECOPD within a period of 12 months (e.g. in the previous 12 months). In another aspect, the present invention provides an immunogenic composition for use, use of an immunogenic composition or method according to the present invention, wherein the subject (e.g. a human aged 40 to 80 years old) has previously been administered a pneumococcal vaccine and/or has been administered an influenza vaccine in the previous 12 months and has experienced at least one severe episode of AECOPD within a period of 12 months (e.g. in the previous 12 months).

Particular Aspects of the Present Invention Include:
An immunogenic composition comprising: (i) an immunogenic polypeptide from non-typeable Haemophilus influenzae, (ii) a PE-PilA fusion protein, (iii) optionally an immunogenic UspA2 polypeptide from Moraxella catarrhalis, and (iv) an adjuvant, for use in reducing the frequency of severe exacerbations, in a subject (e.g. human) having chronic obstructive pulmonary disease (COPD) wherein the subject has previously been administered a pneumococcal vaccine and/or has been administered an influenza vaccine in the previous 12 months.
The use of an immunogenic composition comprising: (i) an immunogenic polypeptide from non-typeable Haemophilus influenzae, (ii) a PE-PilA fusion protein, (iii) optionally an immunogenic UspA2 polypeptide from Moraxella catarrhalis, and (iv) an adjuvant, in the manufacture of a medicament for reducing the frequency of severe exacerbations, in a subject (e.g. human) having chronic obstructive pulmonary disease (COPD) wherein the subject has previously been administered a pneumococcal vaccine and/or has been administered an influenza vaccine in the previous 12 months.
A method for reducing the frequency of severe exacerbations, in a subject (e.g. human) having chronic obstructive pulmonary disease (COPD), said method comprising administering to said subject a therapeutically effective amount of an immunogenic composition comprising: (i) an immunogenic polypeptide from non-typeable Haemophilus influenzae, (ii) a PE-PilA fusion protein, and (iii) optionally an immunogenic UspA2 polypeptide from Moraxella catarrhalis, and (iv) an adjuvant, wherein the subject has previously been administered a pneumococcal vaccine and/or has been administered an influenza vaccine in the previous 12 months.
The immunogenic composition for use, use of an immunogenic composition or method as described above, in a subject (e.g. human) having chronic obstructive pulmonary disease (COPD) wherein the subject has previously been administered a pneumococcal vaccine.
The immunogenic composition for use, use of an immunogenic composition or method as described above, in a subject (e.g. human) having chronic obstructive pulmonary disease (COPD) wherein the subject has previously been administered a pneumococcal vaccine, for reducing the yearly rate of severe exacerbations compared to a subject who has not been administered the immunogenic composition.
The immunogenic composition for use, use of an immunogenic composition or method as described above in a subject (e.g. human) having chronic obstructive pulmonary disease
(COPD) wherein the subject has previously been administered a pneumococcal vaccine, for reducing the rate of severe exacerbations (and related hospitalizations) during the winter season (December-February in Europe).
The immunogenic composition for use, use of an immunogenic composition or method as described above in a subject (e.g. human) having chronic obstructive pulmonary disease (COPD) wherein the subject has previously been administered a pneumococcal vaccine, for reducing the rate of severe exacerbations (and related hospitalizations) during the spring season (March-May in Europe).
The immunogenic composition for use, use of an immunogenic composition or method as described above in a subject (e.g. human) having chronic obstructive pulmonary disease (COPD) wherein the subject has previously been administered a pneumococcal vaccine, for reducing the rate of severe exacerbations (and related hospitalizations) during the summer season (June-August in Europe).
The immunogenic composition for use, use of an immunogenic composition or method as described above in a subject (e.g. human) having chronic obstructive pulmonary disease (COPD) wherein the subject has previously been administered a pneumococcal vaccine, for reducing the rate of severe exacerbations (and related hospitalizations) during the autumn season (September-November in Europe).
The immunogenic composition for use, use of an immunogenic composition or method as described above, in a subject (e.g. human) having chronic obstructive pulmonary disease (COPD) wherein the subject has previously been administered a pneumococcal vaccine and/or has been administered an influenza vaccine in the previous 12 months, and wherein the immunogenic composition comprises an immunogenic polypeptide of Protein D, suitably an isolated immunogenic polypeptide with at least 70%, 80%, 85%, 90%, 91%, 92%, 93%, 94%, 95%, 96%, 97%, 98%, 99%, or 100% to SEQ ID NO: 2.
The immunogenic composition for use, use of an immunogenic composition or method as described above, in a subject (e.g. human) having chronic obstructive pulmonary disease (COPD) wherein the subject has previously been administered a pneumococcal vaccine and/or has been administered an influenza vaccine in the previous 12 months, and wherein the immunogenic composition comprises a PE-PilA fusion protein, suitably an isolated immunogenic polypeptide with at least 70%, 80%, 85%, 90%, 91%, 92%, 93%, 94%, 95%, 96%, 97%, 98%, 99%, or 100% to SEQ ID NO: 9.
The immunogenic composition for use, use of an immunogenic composition or method as described above, in a subject (e.g. human) having chronic obstructive pulmonary disease (COPD) wherein the subject has previously been administered a pneumococcal vaccine and/or has been administered an influenza vaccine in the previous 12 months, and wherein the composition comprises an immunogenic polypeptide of UspA2 from Moraxella catarrhalis, suitably an isolated immunogenic polypeptide with at least 70%, 80%, 85%, 90%, 91%, 92%, 93%, 94%, 95%, 96%, 97%, 98%, 99%, or 100% to a polypeptide selected from the group consisting of MC-001 (SEQ ID NO: 11), MC-002 (SEQ ID NO: 12), MC-003 (SEQ ID NO: 13), MC-004 (SEQ ID NO: 14), MC-005 (SEQ ID NO: 15), MC-006 (SEQ ID NO: 16), MC-007 (SEQ ID NO: 17), MC-008 (SEQ ID NO:18), MC-009 (SEQ ID NO: 19), MC-010 (SEQ ID NO: 20) or MC-011 (SEQ ID NO: 21) e.g. MC009 SEQ ID NO: 19.
The immunogenic composition for use, use of an immunogenic composition or method as described above, in a subject (e.g. human) having chronic obstructive pulmonary disease (COPD) wherein the subject has previously been administered a pneumococcal vaccine and/or has been administered an influenza vaccine in the previous 12 months, and wherein the immunogenic composition comprises a pharmaceutically acceptable excipient or carrier.
The immunogenic composition for use, use of an immunogenic composition or method as described above, in a subject (e.g. human) having chronic obstructive pulmonary disease (COPD) wherein the subject has previously been administered a pneumococcal vaccine and/or has been administered an influenza vaccine in the previous 12 months, and wherein the adjuvant is an AS01 adjuvant, e.g. ASO1E.
   Immunogenic Polypeptides from Non-Typeable H. influenzae (NTHi)

In one aspect of the present invention, the immunogenic composition comprises an immunogenic polypeptide from non-typeable Haemophilus influenzae (H. influenzae, NTHi). In one aspect of the present invention, the immunogenic polypeptide from non-typeable H. influenzae (NTHi) is an immunogenic polypeptide of Protein D from NTHi, suitably an isolated immunogenic polypeptide with at least 70%, 80%, 85%, 90%, 91%, 92%, 93%, 94%, 95%, 96%, 97%, 98%, 99%, or 100% to Protein D sequence. In another aspect of the present invention, from non-typeable H. influenzae (NTHi) is an immunogenic fragment of Protein D from NTHi, suitably an isolated immunogenic polypeptide with at least 70%, 80%, 85%, 90%, 91%, 92%, 93%, 94%, 95%, 96%, 97%, 98%, 99%, or 100% to Protein D sequence.

Protein D is described in WO91/18926. In an embodiment, the immunogenic composition of the present invention comprises Protein D from Haemophilus influenzae (PD), for example, Protein D sequence from FIG. 9 (FIGS. 9a and 9b together, 364 amino acids) of EP 0594610 (SEQ ID NO: 1). Inclusion of this protein in the immunogenic composition may provide a level of protection against Haemophilus influenzae related otitis media (Pyrmula et al Lancet 367; 740-748 (2006)). Protein D may be used as a full length protein or as a fragment (for example, Protein D may be as described in WO0056360). For example, a Protein D immunogenic polypeptide may comprise (or consist) of the Protein D fragment described in EP0594610 which begins at the sequence SSHSSNMANT (SerSerHisSerSerAsnMetAlaAsnThr) (SEQ ID NO: 3), and lacks the 19 N-terminal amino acids from FIG. 9 of EP0594610, optionally with the tripeptide MDP from NS1 fused to the N-terminal of said Protein D fragment (348 amino acids) (SEQ ID NO:2). In one aspect, the Protein D immunogenic polypeptide is unlipidated.

In one aspect of the present invention, the immunogenic composition comprises an immunogenic polypeptide of Protein D from NTHi, suitably an isolated immunogenic polypeptide with at least 70%, 80%, 85%, 90%, 91%, 92%, 93%, 94%, 95%, 96%, 97%, 98%, 99%, or 100% to SEQ ID NO: 1. Immunogenic fragments of Protein D comprise immunogenic fragments of at least 7, 10, 15, 20, 25, 30 or 50 contiguous amino acids of SEQ ID NO: 1. For example, immunogenic fragments of Protein D may comprise immunogenic fragments of at least 7, 10, 15, 20, 25, 30, 50, 100, 200 or 300 contiguous amino acids of SEQ ID NO: 1, up to 363 contiguous amino acids of SEQ ID NO: 1. The Protein D polypeptide sequence (e.g. SEQ ID NO: 1) may be modified by the deletion and/or addition and/or substitution of one or more amino acids (e.g. 1, 2, 3, 4, 5, 6, 7, 8, 9, 10, 11 or 12 amino acids). The immunogenic fragments may elicit antibodies which can bind SEQ ID NO: 1. In another aspect of the present invention, the immunogenic composition comprises a Protein D immunogenic polypeptide, suitably an isolated immunogenic polypeptide with at least 70%, 80%, 85%, 90%, 91%, 92%, 93%, 94%, 95%, 96%, 97%, 98%, 99%, or 100% to SEQ ID NO: 2. Immunogenic fragments of Protein D may comprise at least 7, 10, 15, 20, 25, 30 or 50 contiguous amino acids of SEQ ID NO: 2. Immunogenic fragments of Protein D may comprise 100, 200, 300, 310, 320, 330 or 340 contiguous amino acids of SEQ ID NO: 2. The Protein D polypeptide sequence (e.g. SEQ ID NO: 2) may be modified by the deletion and/or addition and/or substitution of one or more amino acids (e.g. 1, 2, 3, 4, 5, 6, 7, 8, 9, 10, 11 or 12 amino acids). The immunogenic fragments may elicit antibodies which can bind SEQ ID NO: 2.

In one aspect of the present invention, the immunogenic composition comprises an immunogenic polypeptide of Protein E from non-typeable H. influenzae (NTHi), suitably an isolated immunogenic polypeptide with at least 70%, 80%, 85%, 90%, 91%, 92%, 93%, 94%, 95%, 96%, 97%, 98%, 99%, or 100% to Protein E sequence. In another aspect of the present invention, the immunogenic composition comprises an immunogenic fragment of Protein E from non-typeable H. influenzae (NTHi), suitably an isolated immunogenic polypeptide with at least 70%, 80%, 85%, 90%, 91%, 92%, 93%, 94%, 95%, 96%, 97%, 98%, 99%, or 100% to Protein E sequence.

Protein E (PE) is an outer membrane lipoprotein with adhesive properties. It plays a role in the adhesion/invasion of non-typeable Haemophilus influenzae (NTHi) to epithelial cells. (J. Immunology 183: 2593-2601 (2009); The Journal of Infectious Diseases 199:522-531 (2009), Microbes and Infection 10:87-96 (2008)). It is highly conserved in both encapsulated Haemophilus influenzae and non-typeable H. influenzae and has a conserved epithelial binding domain. (The Journal of Infectious Diseases 201:414-419 (2010)). Thirteen different point mutations have been described in different Haemophilus species when compared with Haemophilus influenzae Rd as a reference strain. Its expression is observed on both logarithmic growing and stationary phase bacteria. (WO2007/084053).

Protein E is also involved in human complement resistance through binding vitronectin. (Immunology 183: 2593-2601 (2009)). PE, by the binding domain PKRYARSVRQ YKILNCANYH LTQVR (SEQ ID NO: 1, corresponding to amino acids 84-108 of SEQ ID NO: 4), binds vitronectin which is an important inhibitor of the terminal complement pathway. (J. Immunology 183:2593-2601 (2009)).

As used herein “Protein E”, “protein E”, “Prot E”, and “PE” mean Protein E from non-typeable H. influenzae (NTHi). Protein E may consist of or comprise the amino acid sequence of SEQ ID NO: 4 (corresponding to SEQ ID NO: 4 of WO2012/139225A1): (MKKIILTLSL GLLTACSAQI QKAEQNDVKL APPTDVRSGY IRLVKNVNYY IDSESIWVDN QEPQIVHFDA WNLDKGLYV YPEPKRYARS VRQYKILNCA NYHLTQVRTD FYDEFWGQGL RAAPKKQKKH TLSLTPDTTL YNAAQIICAN YGEAFSVDKK) as well as sequences with at least or exactly 75%, 77%, 80%, 85%, 90%, 95%, 97%, 99% or 100% identity, over the entire length, to SEQ ID NO: 4. In an aspect of the present invention, the immunogenic composition comprises an immunogenic polypeptide of Protein E from non-typeable H. influenzae (NTHi), suitably an isolated immunogenic polypeptide with at least 70%, 80%, 85%, 90%, 91%, 92%, 93%, 94%, 95%, 96%, 97%, 98%, 99%, or 100% to SEQ ID NO: 4. Immunogenic fragments of Protein E comprise immunogenic fragments of at least 7, 10, 15, 20, 25, 30 or 50 contiguous amino acids of SEQ ID NO: 4. For example, immunogenic fragments of Protein E may comprise at least 7, 10, 15, 20, 25, 30, 50, 100 or 150 contiguous amino acids of SEQ ID NO: 4, up to 159 contiguous amino acids of SEQ ID NO: 4. The immunogenic fragments may elicit antibodies which can bind SEQ ID NO: 4.

In another aspect of the present invention, the immunogenic composition comprises an immunogenic polypeptide of Protein E from non-typeable H. influenzae (NTHi), suitably an isolated immunogenic polypeptide with at least 70%, 80%, 85%, 90%, 91%, 92%, 93%, 94%, 95%, 96%, 97%, 98%, 99%, or 100% to SEQ ID NO: 5 (corresponding to SEQ ID NO: 125 of WO2012/139225A1):

SEQ ID NO: 5:
Amino acids 20-160 of Protein E
I QKAEQNDVKL APPTDVRSGY IRLVKNVNYY
IDSESIWVDN QEPQIVHFDA WVNLDKGLYV
YPEPKRYARS VRQYKILNCA NYHLTQVRTD
FYDEFWGQGL RAAPKKQKKH TLSLTPDTTL
YNAAQIICAN YGEAFSVDKK

For example, an immunogenic polypeptide of Protein E may comprise (or consist) of the amino acid sequence of SEQ ID NO: 5.

In one aspect of the present invention, the immunogenic composition comprises an immunogenic polypeptide of PilA from non-typeable H. influenzae (NTHi), suitably an isolated immunogenic polypeptide with at least 70%, 80%, 85%, 90%, 91%, 92%, 93%, 94%, 95%, 96%, 97%, 98%, 99%, or 100% to PilA sequence. In another aspect of the present invention, the immunogenic composition comprises an immunogenic fragment of PilA from non-typeable H. influenzae (NTHi), suitably an isolated immunogenic polypeptide with at least 70%, 80%, 85%, 90%, 91%, 92%, 93%, 94%, 95%, 96%, 97%, 98%, 99%, or 100% to PilA sequence.

Pilin A (PilA) is likely the major pilin subunit of non-typeable H. influenzae (NTHi) Type IV Pilus (Tfp) involved in twitching motility (Infection and Immunity, 73: 1635-1643 (2005)). NTHi PilA is a conserved adhesin expressed in vivo. It has been shown to be involved in NTHi adherence, colonization and biofilm formation. (Molecular Microbiology 65: 1288-1299 (2007)).

As used herein “PilA” means Pilin A from non-typeable H. influenzae (NTHi). PilA may consist of or comprise the amino acid sequence of SEQ ID NO: 6 (corresponding to SEQ ID NO: 58 of WO2012/139225A1) (MKLTTQQTLK KGFTLIELMI VIAIIAILAT IAIPSYQNYT KKAAVSELLQ ASAPYKADVE LCVYSTNETT NCTGGKNGIA ADITTAKGYV KSVTTSNGAI TVKGDGTLAN MEYILQATGN AATGVTWTTT CKGTDASLFP ANFCGSVTQ) as well as sequences with 80% to 100% identity to SEQ ID NO: 6. For example, PilA may be at least 80%, 85%, 90%, 95%, 97% or 100% identical to SEQ ID NO: 6. In an aspect of the present invention, the immunogenic composition comprises an immunogenic polypeptide of PilA from non-typeable H. influenzae (NTHi), suitably an isolated immunogenic polypeptide with at least 70%, 80%, 85%, 90%, 91%, 92%, 93%, 94%, 95%, 96%, 97%, 98%, 99%, or 100% to SEQ ID NO: 6.

For example, immunogenic fragments of PilA comprise immunogenic fragments of at least 7, 10, 15, 20, 25, 30 or 50 contiguous amino acids of SEQ ID NO: 6. For example, immunogenic fragments of PilA may comprise at least 7, 10, 15, 20, 25, 30, 50 or 100 contiguous amino acids of SEQ ID NO: 6, up to 148 contiguous amino acids of SEQ ID NO: 6. The immunogenic fragments may elicit antibodies which can bind SEQ ID NO: 6.

In another aspect of the present invention, the immunogenic composition comprises an immunogenic polypeptide of PilA from non-typeable H. influenzae (NTHi), suitably an isolated immunogenic polypeptide with at least 70%, 80%, 85%, 90%, 91%, 92%, 93%, 94%, 95%, 96%, 97%, 98%, 99%, or 100% to SEQ ID NO: 7 (corresponding to SEQ ID NO: 127 of WO2012/139225A1):

SEQ ID NO: 7
Amino acids 40-149 of PilA
from H. influenzae strain 86-028NP
T KKAAVSELLQ ASAPYKADVE LCVYSTNETT
NCTGGKNGIA ADITTAKGYV KSVTTSNGAI
TVKGDGTLAN MEYILQATGN AATGVTWTTT
CKGTDASLFP ANFCGSVTQ.

For example, an immunogenic polypeptide of PilA may comprise (or consist) of the amino acid sequence of SEQ ID NO: 7.

Protein E and Pilin A may be presented as a PE-PilA fusion protein. A PE-PilA fusion protein comprises both an immunogenic polypeptide of Protein E from non-typeable H. influenzae (NTHi) and an immunogenic polypeptide of PilA from non-typeable H. influenzae (NTHi). In an embodiment, a A PE-PilA fusion protein comprises both an immunogenic fragment of Protein E from non-typeable H. influenzae (NTHi) and an immunogenic fragment of PilA from non-typeable H. influenzae (NTHi).

In an aspect of the present invention, the immunogenic composition comprises an immunogenic polypeptide of Protein E from non-typeable H. influenzae (NTHi) and an immunogenic polypeptide of PilA from non-typeable H. influenzae (NTHi), wherein the immunogenic polypeptides of Protein E and PilA are present as a fusion protein (i.e. a PE-PilA fusion protein), suitably an isolated immunogenic polypeptide with at least 70%, 80%, 85%, 90%, 91%, 92%, 93%, 94%, 95%, 96%, 97%, 98%, 99%, or 100% to LVL-735 SEQ ID NO: 8 (corresponding to SEQ ID NO: 194 of WO2012/139225A1).

SEQ ID NO: 8: LVL735 (protein):
(pelB sp)(ProtE aa 20-160)(GG)
(PilA aa40-149):
MKYLLPTAAA GLLLLAAQPA MAIQKAEQND
VKLAPPTDVR SGYIRLVKNV NYYIDSESIW
VDNQEPQIVH FDAVVNLDKG LYVYPEPKRY
ARSVRQYKIL NCANYHLTQV RTDFYDEFWG
QGLRAAPKKQ KKHTLSLTPD TTLYNAAQII
CANYGEAFSV DKKGGTKKAA VSELLQASAP
YKADVELCVY STNETTNCTG GKNGIAADIT
TAKGYVKSVT TSNGAITVKG DGTLANMEYI
LQATGNAATG VTWTTTCKGT DASLFPANFC
GSVTQ

For example, a PE-PilA fusion protein may comprise (or consist) of the amino acid sequence of SEQ ID NO: 8.

In another aspect of the present invention, the immunogenic composition comprises immunogenic polypeptides of Protein E and PilA, wherein Protein E and PilA are present as a fusion protein (i.e. a PE-PilA fusion protein), suitably an isolated immunogenic polypeptide with at least 70%, 80%, 85%, 90%, 91%, 92%, 93%, 94%, 95%, 96%, 97%, 98%, 99%, or 100% to LVL-735, wherein the signal peptide has been removed, SEQ ID NO: 9 (Corresponding to SEQ ID NO: 219 of WO2012/139225A1).

SEQ ID NO: 9:
PE-PilA fusion protein without
signal peptide
IQKAEQND VKLAPPTDVR SGYIRLVKNV
NYYIDSESIW VDNQEPQIVH FDAVVNLDKG
LYVYPEPKRY ARSVRQYKIL NCANYHLTQV
RTDFYDEFWG QGLRAAPKKQ KKHTLSLTPD
TTLYNAAQII CANYGEAFSV DKKGGTKKAA
VSELLQASAP YKADVELCVY STNETTNCTG
GKNGIAADIT TAKGYVKSVT TSNGAITVKG
DGTLANMEYI LQATGNAATG VTWTTTCKGT
DASLFPANFC GSVTQ

For example, a PE-PilA fusion protein may comprise (or consist) of the amino acid sequence of SEQ ID NO: 9.

The immunogenicity of Protein E (PE) and Pilin A (PilA) polypeptides may be measured as described in WO2012/139225A1; the contents of which are incorporated herein by reference.

In one embodiment, identity is calculated using the Needle program, from the EMBOSS package (Free software; EMBOSS: The European Molecular Biology Open Software Suite). In another embodiment, identity is calculated using the algorithm described by Dufresne et al. in Nature Biotechnology in 2002 (vol. 20, pp. 1269-71) and is used in the software GenePAST (Genome Quest Life Sciences, Inc. Boston, M A, 2021).

Immunogenic Polypeptides from Moraxella catarrhalis

In one aspect of the present invention, the immunogenic composition comprises an immunogenic polypeptide from Moraxella catarrhalis (M. catarrhalis). In another aspect of the present invention, the immunogenic composition comprises an immunogenic polypeptide of UspA2 from M. catarrhalis.

In one aspect of the present invention, the immunogenic composition comprises an immunogenic polypeptide of UspA2 from M. catarrhalis, suitably an isolated immunogenic polypeptide with at least 70%, 80%, 85%, 90%, 91%, 92%, 93%, 94%, 95%, 96%, 97%, 98%, 99%, or 100% to UspA2 sequence. In another aspect of the present invention, the immunogenic polypeptide of UspA2 from Moraxella catarrhalis is an immunogenic fragment of UspA2 from M. catarrhalis, suitably an isolated immunogenic polypeptide with at least 70%, 80%, 85%, 90%, 91%, 92%, 93%, 94%, 95%, 96%, 97%, 98%, 99%, or 100% to UspA2 sequence.

Ubiquitous Surface Protein A2 (UspA2) is a trimeric autotransporter that appears as a lollipop-shared structure in electron micrographs (Hoiczyk et al. EMBO J. 19: 5989-5999 (2000)). It is composed of a N-terminal head, followed by a stalk which ends by an amphipathic helix and a C-terminal membrane domain. (Hoiczyk et al. EMBO J. 19: 5989-5999 (2000)). UspA2 contains a very well conserved domain (Aebi et al., Infection & Immunity 65(11) 4367-4377 (1997)), which is recognized by a monoclonal antibody that was shown protective upon passive transfer in a mouse Moraxella catarrhalis challenge model (Helminnen et al. J Infect Dis. 170(4): 867-72 (1994)).

UspA2 has been shown to interact with host structures and extracellular matrix proteins like fibronectin (Tan et al., J Infect Dis. 192(6): 1029-38 (2005)) and laminin (Tan et al., J Infect Dis. 194(4): 493-7 (2006)), suggesting it can play a role at an early stage of Moraxella catarrhalis infection.

UspA2 also seems to be involved in the ability of Moraxella catarrhalis to resist the bactericidal activity of normal human serum. (Attia AS et al. Infect Immun 73(4): 2400-2410 (2005)). It (i) binds the complement inhibitor C4 bp, enabling Moraxella catarrhalis to inhibit the classical complement system, (ii) prevents activation of the alternative complement pathway by absorbing C3 from serum and (iii) interferes with the terminal stages of the complement system, the Membrane Attack Complex (MAC), by binding the complement regulator protein vitronectin. (de Vries et al., Microbiol Mol Biol Rev. 73(3): 389-406 (2009)).

As used herein “UspA2” means Ubiquitous Surface Protein A2 from Moraxella catarrhalis. UspA2 may consist of or comprise the amino acid sequence of SEQ ID NO: 10 from ATCC 25238:

(SEQ ID NO: 10)
MKTMKLLPLKIAVTSAMIIGLGAASTANAQAKNDITLEDLP
YLIKKIDQNELEADIGDITALEKYLALSQYGNILALEELN
KALEELDEDVGWNONDIANLEDDVETLTKNQNALAEQGEA
IKEDLQGLADFVEGQEGKILQNETSIKKNTQRNLVNGFEI
EKNKDAIAKNNESIEDLYDFGHEVAESIGEIHAHNEAQNE
TLKGLITNSIENTNNITKNKADIQALENNVVEELFNLSGR
LIDQKADIDNNINNIYELAQQQDQHSSDIKTLKKNVEEGL
LELSGHLIDQKTDIAQNQANIQDLATYNELQDQYAQKQTE
AIDALNKASSENTQNIEDLAAYNELQDAYAKQQTEAIDAL
NKASSENTQNIEDLAAYNELQDAYAKQQTEAIDALNKASS
ENTQNIAKNQADIANNINNIYELAQQQDQHSSDIKTLAKA
SAANTDRIAKNKADADASFETLTKNQNTLIEKDKEHDKLI
TANKTAIDANKASADTKFAATADAITKNGNAITKNAKSIT
DLGTKVDGFDSRVTALDTKVNAFDGRITALDSKVENGMAA
QAALSGLFQPYSVGKFNATAALGGYGSKSAVAIGAGYRVN
PNLAFKAGAAINTSGNKKGSYNIGVNYEF

as well as sequences with at least or exactly 63%, 66%, 70%, 72%, 74%, 75%, 77%, 80%, 84%, 85%, 90%, 95%, 96%, 97%, 98%, 99% or 100% identity, over the entire length, to SEQ ID NO: 10.

UspA2 polypeptides may be full length UspA2 or an immunogenic fragment thereof. In particular embodiments, the immunogenic composition comprises an UspA2 polypeptide having at least 70%, 80%, 85%, 90%, 91%, 92%, 93%, 94%, 95%, 96%, 97%, 98%, 99%, or 100% identity to SEQ ID NO: 10. In another embodiment, the immunogenic composition comprises an immunogenic fragment of UspA2 from Moraxella catarrhalis having at least 70%, 80%, 85%, 90%, 91%, 92%, 93%, 94%, 95%, 96%, 97%, 98%, or 99% identity to SEQ ID NO: 10. For example, immunogenic fragments of UspA2 may comprise at least 7, 10, 15, 20, 25, 30 or 50 contiguous amino acids of SEQ ID NO: 10. For example, immunogenic fragments of UspA2 may comprise at least 7, 10, 15, 20, 25, 30, 50, 100, 200, 300, 400, 500 or 600 contiguous amino acids of SEQ ID NO: 10, up to 629 contiguous amino acids of SEQ ID NO: 10. The immunogenic fragments may elicit antibodies which can bind SEQ ID NO: 10.

UspA2 as described in SEQ ID NO: 10 contains a signal peptide (for example, amino acids 1 to 29 of SEQ ID NO: 10), a laminin binding domain (for example, amino acids 30 to 177 of SEQ ID NO: 10), a fibronectin binding domain (for example, amino acids 165 to 318 of SEQ ID NO: 10) (Tan et al. JID 192: 1029-38 (2005)), a C3 binding domain (for example, amino acids 30 to 539 of SEQ ID NO: 10 (WO2007/018463), or a fragment of amino acids 30 to 539 of SEQ ID NO: 10, for example, amino acids 165 to 318 of SEQ ID NO: 1 (Hallstrom T et al. J. Immunol. 186: 3120-3129 (2011)), an amphipathic helix (for example, amino acids 519 to 564 of SEQ ID NO: 10 or amino acids 520-559 of SEQ ID NO:10, identified using different prediction methods) and a C terminal anchor domain (for example, amino acids 576 to 630 amino acids of SEQ ID NO: 10 (Brooks et al., Infection & Immunity, 76(11), 5330-5340 (2008)).

In an embodiment, an immunogenic fragment of UspA2 is the mature polypeptide lacking the signal peptide. In an embodiment, an immunogenic fragment of UspA2 contains a laminin binding domain and a fibronectin binding domain. In an additional embodiment, an immunogenic fragment of UspA2 contains a laminin binding domain, a fibronectin binding domain and a C3 binding domain. In a further embodiment, an immunogenic fragment of UspA2 contains a laminin binding domain, a fibronectin binding domain, a C3 binding domain and an amphipathic helix.

UspA2 amino acid differences have been described for various Moraxella catarrhalis species. See for example, J Bacteriology 181(13):4026-34 (1999), Infection and Immunity 76(11):5330-40 (2008) and PLoS One 7(9):e45452 (2012).

UspA2 may consist of or comprise an amino acid sequence that differs from SEQ ID NO: 10 at any one or more amino acid selected from the group consisting of: AA (amino acid) 30 to 298, AA 299 to 302, AA 303 to 333, AA 334 to 339, AA 349, AA 352 to 354, AA 368 to 403, AA 441, AA 451 to 471, AA 472, AA474 to 483, AA 487, AA 490, AA 493, AA 529, AA 532 or AA 543.

UspA2 may consist of or comprise an amino acid sequence that differs from SEQ ID NO: 10 in that it contains an amino acid insertion in comparison to SEQ ID NO: 10. UspA2 may consists of or comprise an amino acid sequence that differs from SEQ ID NO: 10 at any one of the amino acid differences in SEQ ID NO: 22 through SEQ ID NO: 58. For example, SEQ ID NO: 10 may contain K instead of Q at amino acid 70, Q instead of G at amino acid 135 and/or D instead of N at amino acid 216.

TABLE 3
UspA2 amino acid sequences from 38 strains of Moraxalla catarrhalis
(SEQ ID NO: 10 and SEQ ID NO: 22-SEQ ID NO: 58).

| Strain | UspA2 sequences |
|---|---|
| ATCC 25238 | MKTMKLLPLKIAVTSAMIIGLGAASTANAQAKNDITLEDLPYLIKKIDQNELEADIGDITALEK |
| (SEQ ID | YLALSQYGNILALEELNKALEELDEDVGWNQNDIANLEDDVETLTKNQNALAEQGEAIKEDLQG |
| NO: 10) | LADFVEGQEGKILQNETSIKKNTQRNLVNGFEIEKNKDAIAKNNESIEDLYDFGHEVAESIGEI |
| | HAHNEAQNETLKGLITNSIENTNNITKNKADIQALENNVVEELFNLSGRLIDQKADIDNNINNI |
| | YELAQQQDQHSSDIKTLKKNVEEGLLELSGHLIDQKTDIAQNQANIQDLATYNELQDQYAQKQT |
| | EAIDALNKASSENTQNIEDLAAYNELQDAYAKQQTEAIDALNKASSENTQNIEDLAAYNELQDA |
| | YAKQQTEAIDALNKASSENTQNIAKNQADIANNINNIYELAQQQDQHSSDIKTLAKASAANTDR |
| | IAKNKADADASFETLTKNQNTLIEKDKEHDKLITANKTAIDANKASADTKFAATADAITKNGNA |
| | ITKNAKSITDLGTKVDGFDSRVTALDTKVNAFDGRITALDSKVENGMAAQAALSGLFQPYSVGK |
| | FNATAALGGYGSKSAVAIGAGYRVNPNLAFKAGAAINTSGNKKGSYNIGVNYEF (630 aa) |
| American | MKTMKLLPLKIAVTSAMIIGLGAASTANAQSRDRSLEDIQDSISKLVQDDINTLKQDQQKMNKY |
| 2933 | LLLNQLANTLITDELNNNVIKNTNSIEALGDEIGWLENDIADLEEGVEELTKNQNTLIEKDEEH |
| (SEQ ID | DRLIAQNQADIQTLENNVVEELFNLSGRLIDQEADIAKNNASIEELYDFDNEVAERIGEIHAYT |
| NO: 22) | EEVNKTLENLITNSVKNTDNIDKNKADIDNNINHIYELAQQQDQHSSDIKTLKNNVEEGLLELS |
| | GHLIDQKADLTKDIKALESNVEEGLLDLSGRLLDQKADLTKDIKALESNVEEGLLDLSGRLLDQ |
| | KADIAQNQTDIQDLAAYNELQDQYAQKQTEAIDALNKASSENTQNIEDLAAYNELQDAYAKQQT |
| | EAIDALNKASSENTQNIAKNQADIANNINNIYELAQQQDQHSSDIKTLAKASAANTNRIATAEL |
| | GIAENKKDAQIAKAQANANKTAIDENKASADTKFAATADAITKNGNAITKNAKSITDLGTKVDG |
| | FDGRVTALDTKVNAFDGRITALDSKVENGMAAQAALSGLFQPYSVGKENATAALGGYGSKSAVA |
| | IGAGYRVNPNLAFKAGAAINTSGNKKGSYNIGVNYEF (613 aa) |
| American | MKTMKLLPLKIAVTSALIIGLGAASTANAQQQLQTETFLPNFLSNDNYDLTDPFYHNMILGDTA |
| 2912 | LLDKQDGSQPQLKFYSNDKDSVPDSLLESKLLHEQQLNGFKKGDTIIPLDKDGKPVYQVDYKLD |
| (SEQ ID | GKGKKQKRRQVYSVTTKTATDDDVNSAYSRGILGKVDDLDDEMNFLNHDITSLYDVTANQQDAI |
| NO: 23) | KDLKKGVKGLNKELKELDKEVGVLSRDIGSLNDDVAQNNESIEDLYDESQEVADSIGEIHAHNK |
| | AQNETLQDLITNSVENTNNITKNKADIQALENNVVEELFNLSGRLIDQKADLTKDIKTLESNVE |
| | EGLLELSGHLIDQKADIAKNQADIAQNQANIQDLAAYNELQDAYAKQQTEAIDALNKASSENTQ |
| | NIEDLAAYNELQDAYAKQQTEAIDALNKASSENTQNIAKNQADIANNINNIYELAQQQDQHSSD |
| | IKTLAKASAANTDRIAKNKADADASFETLTKNQNTLIEKDKEHDKLITANKTAIDENKASADTK |
| | FAATADAITKNGNAITKNAKSITDLGTKVDGFDSRVTALDTKVNAFDGRITALDSKVENGMAAQ |
| | AALSGLFQPYSVGKFNATAALGGYGSKSAVAIGAGYRVNPNLAFKAGAAINTSGNKKGSYNIGV |
| | NYEF (644 aa) |
| American | MKTMKLLPLKIAVTSALIVGLGAASTANAQLVERFFPNIFLDKPLAKQHYHNVVVGDTSIVSDL |
| 2908 | QSNSDQLKFYSDDEGLVPDSLLFNKMLHEQLLNGFKEGDTIIPLDENGKPVYKVDYKLDGKEPR |
| (SEQ ID | KVYSVTTKIATAEDVATSSYANGIQKDIDDLYDFDHQVTERLTQHGKTIYRNGERILANEESVQ |
| NO: 24) | YLNKEVQNNIEHIYELAQQQDQHSSDIKTLESNVEKGLLELSGHLIDQKADLTKDIKTLESNVE |
| | EGLLDLSGRLIDQKADLTKDIKTLESNVEEGLLDLSGRLIDQKADIAQNQANIQDLAAYNELQD |
| | QYAQKQTEAIDALNKASSENTQNIEDLAAYNELQDAYAKQQTEAIDALNKASSENTQNIAKNQA |
| | DIANNINNIYELAQQQDQHSSDIKTLAKASAANTNRIATAELGIAENKKDAQIAKAQANANKTA |
| | IDENKASADTKFAATADAITKNGNAITKNAKSITDLGTKVDGFDSRVTALDTKVNAFDGRITAL |
| | DSKVENGMAAQAALSGLFQPYSVGKENATAALGGYGSKSAVAIGAGYRVNPNLAFKAGAAINTS |
| | GNKKGSYNIGVNYEF (591 aa) |
| Finnish | MKTMKLLPLKIAVTSAMIIGLGAASTANAQQQQSRTEIFFPNIFFNENHDELDDAYH |
| 307 | NIILGDTALLDKQDGSQPQLKFYSNDKDSVPDSLLFSKLLHEQQLNGFKKGDTIIPLDKDGKPV |
| (SEQ ID | YQVDYKLDGKGKKQKRRQVYSVTTKTATDDDVNSAYSRGILGKVDDLDDEMNFLNHDITSLYDV |
| NO: 25) | TANQQDAIKGLKKGVKGLNKELKELDKEVGVLSRDIGSLNDDVAQNNESIEDLYDFSQEVADSI |
| | GEIHAHNKAQNETLQDLITNSVENTNNITKNKADIQALENNVVEELFNLSGRLIDQKADLTKDI |
| | KTLESNVEEGLLELSGHLIDQKADIAKNQADIAQNQANIQDLAAYNELQDAYAKQQTEAIDALN |
| | KASSENTQNIEDLAAYNELQDAYAKQQTEAIDALNKASSENTQNIEDLAAYNELQDAYAKQQTE |
| | AIDALNKASSENTQNIAKNQADIANNINNIYELAQQQDQHSSDIKTLAKASAANTDRIAKNKAD |
| | ADASFETLTKNQNTLIEKDKEHDKLITANKTAIDENKASADTKFAATADAITKNGNAITKNAKS |
| | ITDLGTKVDAFDGRVTALDTKVNAFDGRITALDSKVENGMAAQAALSGLFQPYSVGKENATAAL |
| | GGYGSKSAVAIGAGYRVNPNLAFKAGAAINTSGNKKGSYNIGVNYEF (687 aa) |
| Finnish | MKTMKLLPLKIAVTSAMIVGLGMASTANAQQQKSPKTETFLPNIFFNEYADDLDTLYHNMILGD |
| 353 | TAITHDDQYKFYADDATEVPDSLFFNKILHDQLLYGFKEGDKIIPLDENGKPVYKLDKRLENGV |
| (SEQ ID | QKTVYSVTTKTATADDVNSAYSRGIQGDIDDLYEANKENVNRLIEHGDKIFANEESVQYLNREV |
| NO: 26) | QNNIENIHELAQQQDQHSSDIKTLKKNVEKDLLDLSGRLIAQKEDIAQNQTDIQDLATYNELQD |
| | QYAQKQTEAIDALNKASSENTQNIAKNSNHIKTLENNIEEGLLELSGHLIDQKADLTKDIKALE |
| | SNVEEGLLDLSGRLIDQKADIAQNQANIQDLAAYNELQDAYAKQQTEAIDALNKASSENTQNIE |
| | DLAAYNELQDAYAKQQTEAIDALNKASSENTQNIEDLAAYNELQDAYAKQQTEAIDALNKASSE |
| | NTQNIAKNQADIANNINNIYELAQQQDQHSSDIKTLAKASAANTDRIAKNKADADASFETLTKN |
| | QNTLIEKDKEHDKLITANKTAIDANKASADTKFAATADAITKNGNAITKNAKSITDLGTKVDGF |
| | DGRVTALDTKVNALDTKVNAFDGRITALDSKVENGMAAQAALSGLFQPYSVGKFNATAALGGYG |
| | SKSAVAIGAGYRVNPNLAFKAGAAINTSGNKKGSYNIGVNYEF (683 amino acids) |
| Finnish | MKTMKLLPLKIAVTSAMMVGLGMASTANAQQQKSPKTEIFLPNLFDNDNTELTDPLYHNMILGN |
| 358 | TALLTQENQYKFYADDGNGVPDSLLENKILHDQLLHGFKEGGTIIPLDENGKPVYKLDSIVEQG |
| (SEQ ID | KTKTVYSVTTKTATADDVNSAYSRGIQGDIDDLYEANKENVNRLIEHGDKIFANEESVQYLNRE |
| NO: 27) | VQNNIENIHELAQQQDQHSSDIKTLKKNVEKDLLDLSGRLIAQKEDIAQNQTDIQDLATYNELQ |
| | DQYAQKQTEAIDALNKASSENTQNIAKNSNHIKTLENNIEEGLLELSGHLIDQKADLTKDIKAL |
| | ESNVEEGLLDLSGRLIDQKADIAQNQANIQDLAAYNELQDAYAKQQTEAIDALNKASSENTQNI |
| | EDLAAYNELQDAYAKQQTEAIDALNKASSENTQNIEDLAAYNELQDAYAKQQTEAIDALNKASS |
| | ENTQNIAKNQADIANNINNIYELAQQQDQHSSDIKTLAKASAANTDRIAKNKADADASFETLTK |
| | NQNTLIEKDKEHDKLITANKTAIDANKASADTKFAATADAITKNGNAITKNAKSITDLGTKVDG |
| | FDGRVTALDTKVNALDTKVNAFDGRITALDSKVENGMAAQAALSGLFQPYSVGKENATAALGGY |
| | GSKSAVAIGAGYRVNPNLAFKAGAAINTSGNKKGSYNIGVNYEF (684 amino acids) |
| Finnish | MKTMKLLPLKIAVTSAMIIGLGAASTANAQQQQKTKTEVFLPNLFDNDYYDLTDPLYHSMILGD |
| 216 | TATLFDQQDNSKSQLKFYSNDKDSVPDSLLESKLLHEQQLNGFKAGDTIIPLDKDGKPVYTQDT |
| (SEQ ID | RTKDGKVETVYSVTTKIATQDDVEQSAYSRGIQGDIDDLYDINREVNEYLKATHDYNERQTEAI |
| NO: 28) | DALNKASSANTDRIDTAEERIDKNEYDIKALESNVGKDLLDLSGRLIAQKEDIDNNINHIYELA |
| | QQQDQHSSDIKTLKNNVEEGLLELSGHLIDQKADLTKDIKTLENNIEEGLLELSGHLIDQKADL |
| | TKDIKTLENNIEEGLLELSGHLIDQKADIAQNQANIQDLAAYNELQDQYAQKQTEAIDALNKAS |
| | SENTQNIEDLAAYNELQDAYAKQQTEAIDALNKASSENTQNIEDLAAYNELQDAYAKQQTEAID |
| | ALNKASSENTQNIAKNQADIANNINNIYELAQQQDQHSSDIKTLAKVSAANTDRIAKNKADADA |
| | SFETLTKNQNTLIEKDKEHDKLITANKTAIDANKASADTKFAATADAITKNGNAITKNAKSITD |
| | LGTKVDGFDGRVTALDTKVNAFDGRITALDSKVENGMAAQAALSGLFQPYSVGKENATAALGGY |
| | GSKSAVAIGAGYRVNPNLAFKAGAAINTSGNKKGSYNIGVNYEF (684 amino acids) |
| Dutch H2 | MKTMKLLPLKIAVTSAMMVGLGMASTANAQQQKSPKTEIFLPNLFDNDNTELTDPLYHNMILGN |
| (SEQ ID | TALLTQENQYKFYADDGNGVPDSLLENKILHDQLLHGFKKGDTIIPLDENGKPVYKLDSIVEQG |
| NO: 29) | KTKTVYSVTTKTATADDVNSAYSRGIQGDIDDLYEANKENVNRLIEHGDKIFANEESVQYLNRE |
| | VQNNIENIYELVQQQDQHSSDIKTLKKNVEKDLLDLSGRLIAQKEDIAQNQTDIQDLATYNELQ |
| | DQYAQKQTEAIDALNKASSENTQNIAKNSNHIKTLENNIEEGLLELSGHLIDQKADLTKDIKAL |
| | ESNVEEGLLDLSGRLIDQKADIAQNQANIQDLAAYNELQDAYAKQQTEAIDALNKASSENTQNI |
| | EDLAAYNELQDAYAKQQTEAIDALNKASSENTQNIEDLAAYNELQDAYAKQQTEAIDALNKASS |
| | ENTQNIAKNQADIANNINNIYELAQQQDQHSSDIKTLAKASAANTDRIAKNKADADASFETLTK |
| | NQNTLIEKDKEHDKLITANKTAIDANKASADTKFAATADAITKNGNAITKNAKSITDLGTKVDG |
| | FDGRVTALDTKVNALDTKVNAFDGRITALDSKVENGMAAQAALSGLFQPYSVGKENATAALGGY |
| | GSKSAVAIGAGYRVNPNLAFKAGAAINTSGNKKGSYNIGVNYEF (684 amino acids) |
| Dutch F10 | MKTMKLLPLKIAVTSAMIIGLGAASTANAQLAEQFFPNIFSNHAPVKQHYHNVVVGDTSIVENL |
| (SEQ ID | QDSDDTQLKFYSNDEYSVPDSLLFNKMLHEQQLNGFKKGDTIIPLDENGKPVYKVDYKLDGQEP |
| NO: 30) | RRVYSVTTKIATQDDVDNSPYSRGIQGDIDDLYEANKENVNRLIEHGDKIFANEESVQYLNKEV |
| | QNNIENIYELAQQQDQHSSDIKTLKKNVEEGLLELSGHLIDQKADLTKDIKTLESNVEEGLLEL |
| | SGHLIDQKADIAKNQADIAQNQANIQDLAAYNELQDAYAKQQTEAIDALNKASSENTQNIEDLA |
| | AYNELQDAYAKQQTEAIDALNKASSENTQNIAKNQADIANNINNIYELAQQQDQHSSDIKTLAK |
| | ASAANTDRIAKNKADADASFETLTKNQNTLIEKDKEHDKLITANKTAIDANKASADTKFAATAD |
| | AITKNGNAITKNAKSITDLGTKVDAFDGRVTALDTKVNAFDGRITALDSKVENGMAAQAALSGL |
| | FQPYSVGKFNATAALGGYGSKSAVAIGAGYRVNPNLAFKAGAAINTSGNKKGSYNIGVNYEF |
| | (574 amino acids) |
| Norwegian | MKTMKLLPLKIAVTSALIVGLGAASTANAQQQPQTETFFPNIFFNENHDALDDVYHNMILGDTA |
| 1 | ITQDNQYKFYADAISEVPDSLLENKILHDQQLNGFKEGDTIIPLDENGKPVYKLDEKVENGVKK |
| (SEQ ID | SVYSVTTKTATRADVEQSAYSRGIQGDIDDLYEANKENVNRLIEHGDKIFANEESVQYLNKEVQ |
| NO: 31) | NNIENIHELAQQQDQHSSDIKTLKKNVEEGLLELSGHLIDQKADLTKDIKTLESNVEEGLLDLS |
| | GRLLDQKADIAQNQANIQDLAAYNELQDAYAKQQTEAIDALNKASSENTQNIEDLAAYNELQDA |
| | YAKQQTEAIDALNKASSENTQNIEDLAAYNELQDAYAKQQTEAIDALNKASSENTQNIEDLAAY |
| | NELQDAYAKQQTEAIDALNKASSENTQNIEDLAAYNELQDAYAKQQTEAIDALNKASSENTQNI |
| | AKNQADIANNINNIYELAQQQDQHSSDIKTLAKASAANTDRIAKNKADADASFETLTKNQNTLI |
| | EKDKEHDKLITANKTAIDANKASADTKFAATADAITKNGNAITKNAKSITDLGTKVDAFDGRVT |
| | ALDTKVNALDTKVNAFDGRITALDSKVENGMAAQAALSGLFQPYSVGKFNATAALGGYGSKSAV |
| | AIGAGYRVNPNLAFKAGAAINTSGNKKGSYNIGVNYEF (678 amino acids) |
| Norwegian | MKTMKLLPLKIAVTSAMIVGLGAASTANAQQQQQPRTETFFPNIFFNENHDALDDVYHNMILGD |
| 13 | TAITQDNQYKFYADAISEVPDSLLENKILHDQQLNGFKEGDTIIPLDENGKPVYKLDEKVENGV |
| (SEQ ID | KKSVYSVTTKTATRADVEQSAYSRGIQGDIDDLYEANKENVNRLIEHGDKIFANEESVQYLNRE |
| NO: 32) | VQNNIENIHELAQQQDQHSSDIKTLKKNVEKDLLDLSGRLIAQKEDIAQNQTDIQDLATYNELQ |
| | DQYAQKQTEAIDALNKASSENTQNIAKNSNHIKTLENNIEEGLLELSGHLIDQKADLTKDIKTL |
| | ENNIEEGLLELSGHLIDQKADLTKDIKALESNVEEGLLDLSGRLLDQKADIAQNQANIQDLAAY |
| | NELQDQYAQKQTEAIDALNKASSENTQNIEDLAAYNELQDAYAKQQTEAIDALNKASSENTQNI |
| | AKNQADIANNINNIYELAQQQDQHSSDIKTLAKASAANTDRIAKNKADADASFETLTKNQNTLI |
| | EKDKEHDKLITANKTAIDTNKASADTKFAATADAITKNGNAITKNAKSITDLGTKVDGFDGRVT |
| | ALDTKVNALDTKVNAFDGRITALDSKVENGMAAQAALSGLFQPYSVGKENATAALGGYGSKSAV |
| | AIGAGYRVNPNLAFKAGAAINTSGNKKGSYNIGVNYEF (678 amino acids) |
| Norwegian | MKTMKLLPLKIAVTSALIVGLGAASTANAQLVERFFPNIFLDKPLAKQHYHNVVVGDTSIVSDL |
| 33 | QSNSDQLKFYSDDEGLVPDSLLFNKMLHEQLLNGFKEGDTIIPLDENGKPVYKVDYKLDGKEPR |
| (SEQ ID | KVYSVTTKIATAEDVATSSYANGIQKDIDDLYDFDHQVTERLTQHGKTIYRNGERILANEESVQ |
| NO: 33) | YLNKEVQNNIEHIYELAQQQDQHSSDIKTLESNVEKGLLELSGHLIDQKADLTKDIKTLENNVE |
| | EGLLDLSGRLIDQKADIAQNQANIQDLAAYNELQDQYAQKQTEAIDALNKASSENTQNIEDLAA |
| | YNELQDAYAKQQTEAIDALNKASSENTQNIAKNQADIANNINNIYELAQQQDQHSSDIKTLAKA |
| | SAANTDRIAKNKADADASFETLTKNQNTLIEKDKEHDKLITANKTAIDTNKASADTKFAATADA |
| | ITKNGNAITKNAKSITDLGTKVDGFDSRVTALDTKVNALDTKVNALDTKVNAFDGRITALDSKV |
| | ENGMAAQAALSGLFQPYSVGKENATAALGGYGSKSAVAIGAGYRVNPNLAFKAGAAINTSGNKK |
| | GSYNIGVNYEF (587 amino acids) |
| Norwegian | MKTMKLLPLKIAVTSAMIVGLGAASTANAQQQQQPRTETFFPNIFFNENHDALDDVYHNMILGD |
| 25 | TAITQDNQYKFYADAISEVPDSLLENKILHDQQLNGFKEGDTIIPLDENGKPVYKLDEKVENGV |
| (SEQ ID | KKSVYSVTTKTATRADVEQSAYSRGIQGDIDDLYEANKENVNRLIEHGDKIFANEESVQYLNRE |
| NO: 34) | VQNNIENIHELAQQQDQHSSDIKTLKKNVEKDLLDLSGRLIAQKEDIAQNQTDIQDLATYNELQ |
| | DQYAQKQTEAIDALNKASSENTQNIAKNSNHIKTLENNIEEGLLELSGHLIDQKADLTKDIKTL |
| | ENNIEEGLLELSGHLIDQKADLTKDIKALESNVEEGLLDLSGRLLDQKADIAQNQANIQDLAAY |
| | NELQDQYAQKQTEAIDALNKASSENTQNIEDLAAYNELQDAYAKQQTEAIDALNKASSENTQNI |
| | AKNQADIANNINNIYELAQQQDQHSSDIKTLAKASAANTDRIAKNKADADASFETLTKNQNTLI |
| | EKDKEHDKLITANKTAIDTNKASADTKFAATADAITKNGNAITKNAKSITDLGTKVDGFDGRVT |
| | ALDTKVNALDTKVNAFDGRITALDSKVENGMAAQAALSGLFQPYSVGKFNATAALGGYGSKSAV |
| | AIGAGYRVNPNLAFKAGAAINTSGNKKGSYNIGVNYEF (678 amino acids) |
| Norwegian | MKTMKLLPLKIAVTSALIVGLGAASTANAQVRDKSLEDIEALLGKIDISKLEKEKKQQTELQKY |
| 27 | LLLSQYANVLTMEELNKNVEKNTNSIEALGYEIGWLENDIADLEEGVEELTKNQNTLIEKDEEH |
| (SEQ ID | DRLIAQNQADIKTLENNVVEELFNLSDRLIDQEADIAKNNASIEELYDEDNEVAERIGEIHAYT |
| NO: 35) | EEVNKTLEKLITNSVKNTDNIDKNKADIQALENNVEEGLLELSGHLIDQKADLTKDIKALESNV |
| | EEGLLDLSGRLLDQKADIAKNQADIAQNQTDIQDLAAYNELQDQYAQKQTEAIDALNKASSENT |
| | QNIEDLAAYNELQDAYAKQQTEAIDALNKASSENTQNIEDLAAYNELQDAYAKQQTEAIDALNK |
| | ASSENTQNIAKNQADIANNINNIYELAQQQDQHSSDIKTLAKVSAANTDRIAKNKADADASFET |
| | LTKNQNTLIEKDKEHDKLITANKTAIDANKASADTKFAATADAITKNGNAITKNAKSITDLGTK |
| | VDGFDSRVTALDTKVNAFDGRITALDSKVENGMAAQAALSGLFQPYSVGKENATAALGGYGSKS |
| | AVAIGAGYRVNPNLAFKAGAAINTSGNKKGSYNIGVNYEF (616 amino acids) |
| Norwegian | MKTMKLLPLKIAVTSALIVGLGAASTANAQATETFLPNLFDNDYTETTDPLYHGMILGNTAITQ |
| 36 | DTQYKFYAENGNEVPDSLFFNKILHDQQLNGFKEGDTIIPLDENGKPVYKLDEITENGVKRKVY |
| (SEQ ID | SVTTKTATREDVEQSAYSRGIQGDIDDLYEANKENVNRLIEHGDKIFANEESVQYLNKEVQNNI |
| NO: 36) | ENIHELAQQQDQHSSDIKTLKKNVEEGLLELSGHLIDQKADLTKDIKALESNVEEGLLDLSGHL |
| | IDQKADLTKDIKALESNVEEGLLDLSGRLLDQKADIAKNQADIAQNQTDIQDLAAYNELQDQYA |
| | QKQTEAIDALNKASSENTQNIEDLAAYNELQDQYAQKQTEAIDALNKASSENTQNIEDLAAYNE |
| | LQDQYAQKQTEAIDALNKASSENTQNIEDLAAYNELQDQYAQKQTEAIDALNKASSENTQNIAK |
| | NQADIANNINNIYELAQQQDQHSSDIKTLAKASAANTDRIAKNKADADASFETLTKNQNTLIEK |
| | DKEHDKLITANKTAIDANKASADTKFAATADAITKNGNAITKNAKSITDLGTKVDGFDGRVTAL |
| | DTKVNALDTKVNAFDGRITALDSKVENGMAAQAALSGLFQPYSVGKFNATAALGGYGSKSAVAI |
| | GAGYRVNPNLAFKAGAAINTSGNKKGSYNIGVNYEF (676 amino acids) |
| BC5SV | MKTMKLLPLKIAVTSALIVGLGAASTANAQNGTSTKLKNLKEYAQYLDNYAQYLDDDIDDL |
| (SEQ ID | DKEVGELSQNIAKNQANIKDLNKKLSRDIDSLREDVYDNQYEIVNNQADIEKNQDDIKELE |
| NO: 37) | NNVGKELLNLSGRLLDQKADIDNNINNIYELAQQQDQHSSDIKTLKKNVEEGLLELSGHLI |
| | DQKSDIAQNQTDIQDLATYNELQDQYAQKQTEAIDALNKASSENTQNIEDLAAYNELQDAY |
| | AKQQTEAIDALNKASSENTQNIQDLAAYNELQDAYAKQQTEAIDALNKASSENTQNIEDLA |
| | AYNELQDAYAKQQTEAIDALNKASSENTQNIEDLAAYNELQDAYAKQQTEAIDALNKASSE |
| | NTQNIEDLAAYNELQDAYAKQQTEAIDALNKASSENTQNIAKNQADIANNINNIYELAQQQ |
| | DQHSSDIKTLAKASAANTDRIAKNKADADASFETLTKNQNTLIEKDKEHDKLITANKTAID |
| | ANKASADTKFAATADAITKNGNAITKNAKSITDLGTKVDAFDGRVTALDTKVNAFDGRITA |
| | LDSKVENGMAAQAALSGLFQPYSVGKFNATAALGGYGSKSAVAIGAGYRVNPNLAFKAGAA |
| | INTSGNKKGSYNIGVNYEF (629 amino acids) |
| Norwegian | MKTMKLLPLKIAVTSAMIVGLGMASTANAQQQRSPKTETFLPNIFFNEYADDLDTLYHNMI |
| 14 | LGDTAITHDDQYKFYADDATEVPDSLFENKILHDQLLYGFKEGDKIIPLDENGKPVYKLDK |
| (SEQ ID | RLDNGVQKTVYSVTTKTATADDVNSAYSRGIQGDIDDLYEANKENVNRLIEHGDKIFANEE |
| NO: 38) | SVQYLNKEVQNNIENIHELAQQQDQHSSDIKTLKKNVEEGLLELSGHLIDQKTDIAQNQTD |
| | IQDLATYNELQDQYAQKQTEAIDALNKASSENTQNIAKNSNRIKALENNIEEGLLELSGHL |
| | IDQKADLTKDIKALESNVEEGLLDLSGRLIDQKADIAQNQANIQDLAAYNELQDAYAKQQT |
| | EAIDALNKASSENTQNIEDLAAYNELQDAYAKQQTEAIDALNKASSENTQNIEDLAAYNEL |
| | QDAYAKQQTEAIDALNKASSENTQNIAKNQADIANNINNIYELAQQQDQHSSDIKTLAKAS |
| | AANTDRIAKNKADADASFETLTKNQNTLIEKDKEHDKLITANKTAIDANKASADTKFAATA |
| | DAITKNGNAITKNAKSITDLGTKVDGFDGRVTALDTKVNALDTKVNAFDGRITALDSKVEN |
| | GMAAQAALSGLFQPYSVGKFNATAALGGYGSKSAVAIGAGYRVNPNLAFKAGAAINTSGNK |
| | KGSYNIGVNYEF (683 amino acids) |
| Norwegian | MKTMKLLPLKIAVTSAMIVGLGAASTANAQAQSNRSLDQVQALLRGIDETKIKKEIQQSQQ |
| 3 | PELNKYLTFNQLANALNIEELNNNVQKNTQRLDSAATLYGDLSKTVPKSIKENKESIKENK |
| (SEQ ID | ESIKENKESIKENKESIKENKESIKENKESITTLTRKSFQNQVDIVRNNASIEDLYAYGQE |
| NO: 39) | VAKSIGEIHAYTEEVNKTLENLITNSVENTNNITKNKADIQALENNVVEELFNLSGRLIDQ |
| | KADIDNNINNIYELAQQQDQHSSDIKTLKKNVEEGLLELSGHLIDQKADLTKDIKTLESNV |
| | EEGLLDLSGRLLDQKADIAQNQANIQDLAAYNELQDAYAKQQTEAIDALNKASSENTQNIE |
| | DLAAYNELQDAYAKQQTEAIDALNKASSENTQNIEDLAAYNELQDAYAKQQTEAIDALNKA |
| | SSENTQNIEDLAAYNELQDAYAKQQTEAIDALNKASSENTQNIAKNQADIANNINNIYELA |
| | QQQDQHSSDIKTLAKASAANTDRIAKNKADADASFETLTKNQNTLIEKDKEHDKLITANKT |
| | VIDANKASADTKFAATADAITKNGNAITKNAKSITDLGTKVDGFDGRVTALDTKVNALDTK |
| | VNAFDGRITALDSKVENGMAAQAALSGLFQPYSVGKFNATAALGGYGSKSAVAIGAGYRVN |
| | PNLAFKAGAAINTSGNKKGSYNIGVNYEF (700 amino acids) |
| Finnish | MKTMKLLPLKIAVTSALIVGLGAASTANAQATETFLPNLFDNDYIETTDPLYHGMILGNTA |
| 414 | ITQDTQYKFYAENGNEVPDSLFFNKILHDQQLNGFKEGDTIIPLDENGKPVYKLDEITENG |
| (SEQ ID | VKRKVYSVTTKTATREDVEQSAYSRGIQGDIDDLYEANKENVNRLIEHGDKIFANEESVQY |
| NO: 40) | LNKEVQNNIENIHELAQQQDQHSSDIKTLKKNVEEGLLELSGHLIDQKADLTKDIKTLENN |
| | VEEGLLELSGHLIDQKADLTKDIKALESNVEEGLLDLSGRLLDQKADIAKNQADIAQNQTD |
| | IQDLAAYNELQDQYAQKQTEAIDALNKASSENTQNIEDLAAYNELQDQYAQKQTEAIDALN |
| | KASSENTQNIEDLAAYNELQDQYAQKQTEAIDALNKASSENTQNIEDLAAYNELQDQYAQK |
| | QTEAIDALNKASSENTQNIAKNQADIANNINNIYELAQQQDQHSSDIKTLAKASAANTDRI |
| | AKNKADADASFETLTKNQNTLIEKDKEHDKLITANKTAIDANKASADTKFAATADAITKNG |
| | NAITKNAKSITDLGTKVDGFDGRVTALDTKVNALDTKVNAFDGRITALDSKVENGMAAQAA |
| | LSGLFQPYSVGKFNATAALGGYGSKSAVAIGAGYRVNPNLAFKAGAAINTSGNKKGSYNIG |
| | VNYEF (676 amino acids) |
| Japanese | MKTMKLLPLKIAVTSAMIIGLGAASTANAQLAEQFFPNIFSNHAPVKQHYHNVVVGDTSIV |
| Z7476 | ENLQDSDDTQLKFYSNDEYSVPDSLLFNKMLHEQQLNGFKKGDTIIPLDENGKPVYKVDYK |
| (SEQ ID | LDGQEPRRVYSVTTKIATQDDVDNSPYSRGIQGDIDDLYEANKENVNRLIEHGDKIFANEE |
| NO: 41) | SVQYLNKEVQNNIENIYELAQQQDQHSSDIKTLKKNVEEGLLELSGRLIDQKADIAQNQAN |
| | IQDLAAYNELQDQYAQKQTEAIDALNKASSENTQNIEDLAAYNELQDAYAKQQTEAIDALN |
| | KASSENTQNIEDLAAYNELQDAYAKQQTEAIDALNKASSENTQNIEDLAAYNELQDAYAKQ |
| | QTEAIDALNKASSENTQNIEDLAAYNELQDAYAKQQTEAIDALNKASSENTQNIEDLAAYN |
| | ELQDAYAKQQTEAIDALNKASSENTQNIAKNQADIANNINNIYELAQQQDQHSSDIKTLAK |
| | VSAANTDRIAKNKADADASFETLTKNQNTLIEKDKEHDKLITANKTAIDANKASADTKFAA |
| | TADAITKNGNAITKNAKSITDLGTKVDGFDGRVTALDTKVNAFDGRITALDSKVENGMAAQ |
| | AALSGLFQPYSVGKFNATAALGGYGSKSAVAIGAGYRVNPNLAFKAGAAINTSGNKKGSYN |
| | IGVNYEF (678 amino acids) |
| Belgian | MKTMKLLPLKIAVTSAMIIGLGAASTANAQSRDRSLEDIQDSISKLVQDDINTLKQDQQKM |
| Z7530 | NKYLLLNQLANTLITDELNNNVIKNTNSIEALGDEIGWLENDIADLEEGVEELTKNQNTLI |
| (SEQ ID | EKDEEHDRLIAQNQADIQTLENNVVEELFNLSGRLIDQEADIAKNNASIEELYDEDNEVAE |
| NO: 42) | RIGEIHAYTEEVNKTLENLITNSVKNTDNIDKNKADIDNNINHIYELAQQQDQHSSDIKTL |
| | KNNVEEGLLELSGHLIDQKADLTKDIKALESNVEEGLLDLSGRLLDQKADLTKDIKALESN |
| | VEEGLLDLSGRLLDQKADIAQNQTDIQDLAAYNELQDQYAQKQTEAIDALNKASSENTQNI |
| | EDLAAYNELQDAYAKQQTEAIDALNKASSENTQNIAKNQADIANNINNIYELAQQQDQHSS |
| | DIKTLAKASAANTNRIATAELGIAENKKDAQIAKAQANANKTAIDENKASADTKFAATADA |
| | ITKNGNAITKNAKSITDLGTKVDGFDGRVTALDTKVNAFDGRITALDSKVENGMAAQAALS |
| | GLFQPYSVGKFNATAALGGYGSKSAVAIGAGYRVNPNLAFKAGAAINTSGNKKGSYNIGVN |
| | YEF (613 amino acids) |
| German | MKTMKLLPLKIAVTSALIVGLGAASTANAQATNKDITLEDVLKSIEEIDPYELRDYIEYPT |
| Z8063 | AIERFLLLSQYGNTLTLEEFDNDIELLDQDVEDLEESVTELAKNQNSLIEQGEAIKEDLQG |
| (SEQ ID | LADFVERQEDKILQNETSIKKNTQRNLVNGFEIEKNKDAIAKNNESIEDLYDFGHEVAKSI |
| NO: 43) | GEIHAHNEAQNETLKDLITNSVKNTDNITKNKADIQALESNVEKGLLELSGHLIDQKADID |
| | NNINNIHELAQQQDQHSSDIKTLKKNVEEGLLELSGHLIDQKSDIAQNQANIQDLATYNEL |
| | QDQYAQKQTEAIDALNKASSENTQNIEDLAAYNELQDAYAKQQTEAIDALNKASSENTQNI |
| | AKNQADIANNINNIYELAQQQDQHSSDIKTLAKASAANTDRIAKNKADADASFETLTKNQN |
| | TLIEKDKEHDKLITANKTAIDANKASADTKFAATADAITKNGNAITKNAKSITDLGTKVDG |
| | FDSRVTALDTKVNAFDGRITALDSKVENGMAAQAALSGLFQPYSVGKFNATAALGGYGSKS |
| | AVAIGAGYRVNPNLAFKAGAAINTSGNKKGSYNIGVNYEF (589 amino acids) |
| American | MKTMKLLPLKIAVTSAMMVGLGMASTANAQQQKSPKTEIFLPNLFDNDNTELTDPLYHNMI |
| O12E | LGNTALLTQENQYKFYADDGNGVPDSLLENKILHDQLLHGFKEGDTIIPLDENGKPVYKLD |
| (SEQ ID | SIVEQGKTKTVYSVTTKTATADDVNSAYSRGIQGDIDDLYEANKENVNRLIEHGDKIFANE |
| NO: 44) | ESVQYLNREVQNNIENIHELAQQQDQHSSDIKTLKKNVEKDLLDLSGRLIAQKEDIAQNQT |
| | DIQDLATYNELQDQYAQKQTEAIDALNKASSENTQNIAKNSNHIKTLENNIEEGLLELSGH |
| | LIDQKADLTKDIKALESNVEEGLLDLSGRLIDQKADIAQNQANIQDLAAYNELQDAYAKQQ |
| | TEAIDALNKASSENTQNIEDLAAYNELQDAYAKQQTEAIDALNKASSENTQNIEDLAAYNE |
| | LQDAYAKQQTEAIDALNKASSENTQNIAKNQADIANNINNIYELAQQQDQHSSDIKTLAKA |
| | SAANTDRIAKNKADADASFETLTKNQNTLIEKDKEHDKLITANKTAIDANKASADTKFAAT |
| | ADAITKNGNAITKNAKSITDLGTKVDGFDGRVTALDTKVNALDTKVNAFDGRITALDSKVE |
| | NGMAAQAALSGLFQPYSVGKFNATAALGGYGSKSAVAIGAGYRVNPNLAFKAGAAINTSGN |
| | KKGSYNIGVNYEF (684 amino acids) |
| Greek | MKTMKLLPLKIAVTSALIVGLGAASTANAQQQQKTKTEVFLPNLFYNDYIEETDLLYHNMI |
| MC317 | LGDTAALVDRQNYSNSQLKFYSNDEESVPDSLLFSKMLNNQQLNGFKAGDIIIPVDANGQV |
| (SEQ ID | IYQKDTRVEGGKTRTVLSVTTKIATQQDVDSAYSRGIQGKVNDLDDEMNFLNHDITSLYDV |
| NO: 45) | TANQQDDIKGLKKGVKDLKKGVKGLNKELKELDKEVGVLSRDIGSLNDDVAQNNESIEDLY |
| | DESQEVADSIGEIHAHNKAQNETLQDLITNSVENTNNITKNKADIQALENNVVEELFNLSG |
| | RLIDQKADLTKDIKTLESNVEEGLLELSGHLIDQKADIAKNQADIAQNQANIQDLAAYNEL |
| | QDAYAKQQTEAIDALNKASSENTQNIEDLAAYNELQDAYAKQQTEAIDALNKASSENTQNI |
| | AKNQADIANNINNIYELAQQQDQHSSDIKTLAKASAANTDRIAKNKADADASFETLTKNQN |
| | TLIEKDKEHDKLITANKTAIDENKASADTKFAATADAITKNGNAITKNAKSITDLGTKVDG |
| | FDGRVTALDTKVNAFDGRITALDSKVENGMAAQAALSGLFQPYSVGKENATAALGGYGSKS |
| | AVAIGAGYRVNPNLAFKAGAAINTSGNKKGSYNIGVNYEF (650 amino acids) |
| American | MKTMKLLPLKIAVTSALIVGLGAVSTTNAQAQSRSLDQIQTKLADLAGKIAAGKNGGGQNN |
| V1122 | QNNQNDINKYLFLSQYANILTMEELNNNVVKNSSSIETLETDFGWLENDVADLEDGVEELT |
| (SEQ ID | KNQNTLIEKDEEHDRLIAQNQADIQTLENNVVEELENLSDRLIDQKADIAKNQADIAQNNE |
| NO: 46) | SIEELYDFDNEVAEKIGEIHAYTEEVNKTLQDLITNSVKNTDNIDKNKADIDNNINHIYEL |
| | AQQQDQHSSDIKTLKNNVEEGLLELSGHLIDQKADLTKDIKTLENNVEEGLLDLSGRLIDQ |
| | KADIAKNQADIAQNQTDIQDLAAYNELQDQYAQKQTEAIDALNKASSENTQNIEDLAAYNE |
| | LQDAYAKQQTEAIDALNKASSENTQNIAKNQADIANNINNIYELAQQQDQHSSDIKTLAKA |
| | SAANTDRIAKNKADADASFETLTKNQNTLIEKDKEHDKLITANKTAIDENKASADTKFAAT |
| | ADAITKNGNAITKNAKSITDLGTKVDGFDGRVTALDTKVNAFDGRITALDSKVENGMAAQA |
| | ALSGLFQPYSVGKFNATAALGGYGSKSAVAIGAGYRVNPNLAFKAGAAINTSGNKKGSYNI |
| | GVNYEF (616 amino acids) |
| American | MKTMKLLPLKIAVTSALIVGLGTASTANAQVASPANQKIQQKIKKVRKELRQDIKSLRNDI |
| P44 | DSNTADIGSLNDDVADNQDDILDNQADIAKNQDDIEKNQADIKELDKEVGVLSREIGSLND |
| (SEQ ID | DIADNYTDIIDNYTDIIDNQANIAKNQDDIEKNQADIKELDKEVGVLSREIGSLNDDVADN |
| NO: 47) | QDDIAKNQADIQTLENNVEEGLLELSGHLLDQKADIDNNINNIYELAQQQDQHSSDIKTLK |
| | KNVEEGLLELSGHLIDQKTDIAQNQANIQDLATYNELQDQYAQEQTEAIDALNKASSENTQ |
| | NIAKNSNRIKALESNVEEGLLELSGHLIDQKADLTKDIKALESNVEEGLLELSGHLIDQKA |
| | DIAQNQANIQDLAAYNELQDQYAQKQTEAIDALNKASSENTQNIEDLAAYNELQDAYAKQQ |
| | TEAIDALNKASSENTQNIAKNQADIANNINNIYELAQQQDQHSSDIKTLAKASAANTDRIA |
| | KNKADADASFETLTKNQNTLIEKDKEHDKLITANKTAIDANKVSADTKFAATADAITKNGN |
| | AITKNAKSITDLGTKVDAFDSRVTALDTKVNAFDGRITALDSKVENGMAAQAALSGLFQPY |
| | SVGKFNATAALGGYGSKSAVAIGAGYRVNPNLAFKAGAAINTSGNKKGSYNIGVNYEF |
| | (668 amino acids) |
| American | MKTMKLLPLKIAVTSAMIVGLGATSTVNAQVVEQFFPNIFFNENHDELDDAYHNMILGDTA |
| V1171 | IVSNSQDNSTQLKFYSNDEDSVPDSLLFSKLLHEQQLNGFKAGDTIIPLDKDGKPVYTKDT |
| (SEQ ID | RTKDGKVETVYSVTTKIATQDDVEQSAYSRGIQGDIDDLYDINREVNEYLKATHDYNERQT |
| NO: 48) | EAIDALNKASSANTDRIDTAEERIDKNEYDIKALESNVEEGLLELSGHLIDQKADLTKDIK |
| | ALESNVEEGLLELSGHLIDQKADLTKDIKALESNVEEGLLDLSGRLIDQKADIAQNQANIQ |
| | DLAAYNELQDAYAKQQTEAIDALNKASSENTQNIEDLAAYNELQDAYAKQQTEAIDALNKA |
| | SSENTQNIEDLAAYNELQDAYAKQQTEAIDALNKASSENTQNIEDLAAYNELQDAYAKQQT |
| | EAIDALNKASSENTQNIAKNQADIANNINNIYELAQQQDQHSSDIKTLAKASAANTDRIAK |
| | NKADADASFETLTKNQNTLIEKDKEHDKLITANKTAIDANKASADTKFAATADAITKNGNA |
| | ITKNAKSITDLGTKVDGFDGRVTALDTKVNALDTKVNAFDGRITALDSKVENGMAAQAALS |
| | GLFQPYSVGKFNATAALGGYGSKSAVAIGAGYRVNPNLAFKAGAAINTSGNKKGSYNIGVN |
| | YEF (674 amino acids) |
| American | MKTMKLLPLKIAVTSAMIIGLGAASTANAQSRDRSLEDIQDSISKLVQDDIDTLKQDQQKM |
| TTA24 | NKYLLLNQLANTLITDELNNNVIKNTNSIEALGDEIGWLENDIADLEEGVEELTKNQNTLI |
| (SEQ ID | EKDEEHDRLIAQNQADIQTLENNVVEELFNLSGRLIDQEADIAKNNASIEELYDEDNEVAE |
| NO: 49) | RIGEIHAYTEEVNKTLENLITNSVKNTDNIDKNKADIDNNINHIYELAQQQDQHSSDIKTL |
| | KNNVEEGLLELSGHLIDQKADLTKDIKALESNVEEGLLDLSGRLLDQKADLTKDIKALESN |
| | VEEGLLDLSGRLLDQKADIAQNQTDIQDLAAYNELQDQYAQKQTEAIDALNKASSENTQNI |
| | EDLAAYNELQDAYAKQQTEAIDALNKASSENTQNIAKNQADIANNINNIYELAQQQDQHSS |
| | DIKTLAKASAANTNRIATAELGIAENKKDAQIAKAQANANKTAIDENKASADTKFAATADA |
| | ITKNGNAITKNAKSITDLGTKVDGFDGRVTALDTKVNAFDGRITALDSKVENGMAAQAALS |
| | GLFQPYSVGKFNATAALGGYGSKSAVAIGAGYRVNPNLAFKAGAAINTSGNKKGSYNIGVN |
| | YEF (613 amino acids) |
| American | MKTMKLLPLKIAVTSAMIVGLGATSTVNAQVVEQFFPNIFFNENHDELDDAYHNMILGDTA |
| 035E | IVSNSQDNSTQLKFYSNDEDSVPDSLLESKLLHEQQLNGFKAGDTIIPLDKDGKPVYTKDT |
| (SEQ ID | RTKDGKVETVYSVTTKIATQDDVEQSAYSRGIQGDIDDLYDINREVNEYLKATHDYNERQT |
| NO: 50) | EAIDALNKASSANTDRIDTAEERIDKNEYDIKALESNVEEGLLELSGHLIDQKADLTKDIK |
| | ALESNVEEGLLELSGHLIDQKADLTKDIKALESNVEEGLLDLSGRLLDQKADIAKNQADIA |
| | QNQTDIQDLAAYNELQDAYAKQQTEAIDALNKASSENTQNIAKNQADIANNINNIYELAQQ |
| | QDQHSSDIKTLAKASAANTDRIAKNKADADASFETLTKNQNTLIEKDKEHDKLITANKTAI |
| | DANKASADTKFAATADAITKNGNAITKNAKSITDLGTKVDGFDGRVTALDTKVNALDTKVN |
| | AFDGRITALDSKVENGMAAQAALSGLFQPYSVGKFNATAALGGYGSKSAVAIGAGYRVNPN |
| | LAFKAGAAINTSGNKKGSYNIGVNYEF (576 amino acids) |
| American | MKTMKLLPLKIAVTSAMMVGLGMASTANAQQQKSPKTEIFLPNLFDNDNTELTDPLYHNMI |
| SP12-6 | LGNTALLTQENQYKFYADDGNGVPDSLLENKILHDQLLHGFKEGDTIIPLDENGKPVYKLD |
| (SEQ ID | SIVEQGKTKTVYSVTTKTATADDVNSAYSRGIQGDIDDLYEANKENVNRLIEHGDKIFANE |
| NO: 51) | ESVQYLNREVQNNIENIHELAQQQDQHSSDIKTLKKNVEKDLLDLSGRLIAQKEDIAQNQT |
| | DIQDLATYNELQDQYAQKQTEAIDALNKASSENTQNIAKNSNHIKTLENNIEEGLLELSGH |
| | LIDQKADLTKDIKALESNVEEGLLDLSGRLIDQKADIAQNQANIQDLAAYNELQDAYAKQQ |
| | TEAIDALNKASSENTQNIEDLAAYNELQDAYAKQQTEAIDALNKASSENTQNIEDLAAYNE |
| | LQDAYAKQQTEAIDALNKASSENTQNIAKNQADIANNINNIYELAQQQDQHSSDIKTLAKA |
| | SAANTDRIAKNKADADASFETLTKNQNTLIEKDKEHDKLITANKTAIDANKASADTKFAAT |
| | ADAITKNGNAITKNAKSITDLGTKVDGFDGRVTALDTKVNALDTKVNAFDGRITALDSKVE |
| | NGMAAQAALSGLFQPYSVGKFNATAALGGYGSKSAVAIGAGYRVNPNLAFKAGAAINTSGN |
| | KKGSYNIGVNYEF (684 amino acids) |
| American | MKTMKLLPLKIAVTSAMIIGLGAASTANAQATETFLPNLFDNDYTETTDPLYHGMILGNTA |
| SP12-5 | ITQDTQYKFYAENGNEVPDSLFFNKILHDQQLNGFKEGDTIIPLDENGKPVYKLDEITENG |
| (SEQ ID | VKRKVYSVTTKTATREDVEQSAYSRGIQGDIDDLYEANKENVNRLIEHGDKIFANEESVQY |
| NO: 52) | LNKEVQNNIENIHELAQQQDQHSSDIKTLKKNVEEGLLELSGRLIAQKEDIAQNQTDIQDL |
| | ATYNELQDQYAQKQTEAIDALNKASSENTQNIAKNSNHIKTLENNIEEGLLELSGHLIDQK |
| | ADLTKDIKALESNVEEGLLDLSGRLLDQKADIAKNQADIAQNQTDIQDLAAYNELQDQYAQ |
| | KQTEAIDALNKASSENTQNIEDLAAYNELQDAYAKQQTEAIDALNKASSENTQNIEDLAAY |
| | NELQDAYAKQQTEAIDALNKASSENTQNIAKNQADIANNINNIYELAQQQDQHSSDIKTLA |
| | KASAANTDRIAKNKADADASFETLTKNQNTLIEKDKEHDKLITANKTAIDANKASADTKFA |
| | ATADAITKNGNAITKNAKSITDLGTKVDGFDGRVTALDTKVNALDTKVNAFDGRITALDSK |
| | VENGMAAQAALSGLFQPYSVGKFNATAALGGYGSKSAVAIGAGYRVNPNLAFKAGAAINTS |
| | GNKKGSYNIGVNYEF (686 amino acids) |
| Swedish | MKTMKLLPLKIAVTSAMIIGLGAASTANAQAKNDITLEDLPYLIKKIDQNELEADIGDITALEK |
| BC5 | YLALSQYGNILALEELNKALEELDEDVGWNQNDIANLEDDVETLTKNQNALAEQGEAIKEDLQG |
| (SEQ ID | LADFVEGQEGKILQNETSIKKNTQRNLVNGFEIEKNKDAIAKNNESIEDLYDFGHEVAESIGEI |
| NO: 53) | HAHNEAQNETLKGLITNSIENTNNITKNKADIQALENNVVEELENLSGRLIDQKADIDNNINNI |
| | YELAQQQDQHSSDIKTLKKNVEEGLLELSGHLIDQKTDIAQNQANIQDLATYNELQDQYAQKQT |
| | EAIDALNKASSENTQNIEDLAAYNELQDAYAKQQTEAIDALNKASSENTQNIEDLAAYNELQDA |
| | YAKQQTEAIDALNKASSENTQNIAKNQADIANNINNIYELAQQQDQHSSDIKTLAKASAANTDR |
| | IAKNKADADASFETLTKNQNTLIEKDKEHDKLITANKTAIDANKASADTKFAATADAITKNGNA |
| | ITKNAKSITDLGTKVDGFDSRVTALDTKVNAFDGRITALDSKVENGMAAQAALSGLFQPYSVGK |
| | FNATAALGGYGSKSAVAIGAGYRVNPNLAFKAGAAINTSGNKKGSYNIGVNYEF (630 |
| | amino acids) |
| American | MKTMKLLPLKIAVTSALIVGLGAASTANAQAQDRSLEQIQDKLANLVEKIEQAKSQNGQSQ |
| 7169 | KDINQYLLLSQYANVLTMEELNNNVVKNSSSIETLDNDIAWLNDDLIDLDKEVGVLSRDIG |
| (SEQ ID | SLHDDVAQNQADIKTLKNNVVEELFNLSDRLIDQEADIAQNNESIEDLYDFGREVAESIGE |
| NO: 54) | IHAHNEAQNETLKDLITNSVKNTDNITKNKADIQALENDVGKELLNLSGRLIDQKADIDNN |
| | INHIYELAQQQDQHSSDIKTLKNNVEEGLLELSGHLIDQKADLTKDIKALESNVEEGLLDL |
| | SGRLLDQKADIAQNQANIQDLAAYNELQDAYAKQQTEAIDALNKASSENTQNIEDLAAYNE |
| | LQDAYAKQQTEAIDALNKASSENTQNIAKNQADIANNINNIYELAQQQDQHSSDIKTLAKA |
| | SAANTDRIAKNKADADASFETLTKNQNTLIEKDKEHDKLITANKTAIDANKASADTKFAAT |
| | ADAITKNGNAITKNAKSITDLGTKVDGFDSRVTALDTKVNAFDGRITALDSKVENGMAAQA |
| | ALSGLFQPYSVGKENATAALGGYGSKSAVAIGAGYRVNPNLAFKAGAAINTSGNKKGSYNI |
| | GVNYEF (616 amino acids) |
| Finnish | MKTMKLLPLKIAVTSAMIIGLGATSTVNAQVVEQFFPNIFFNENHDELDDAYHNMILGDTA |
| FIN2344 | IVSNSQDNSTQLKFYSNDEDSVPDSLLESKLLHEQQLNGFKAGDTIIPLDKDGKPVYTKDT |
| (SEQ ID | RTKDGKVETVYSVTTKIATQDDVEQSAYSRGIQGDIDDLYDINREVNEYLKATHDYNERQT |
| NO: 55) | EAIDALNKASSANTDRIDTAEERIDKNEYDIKALESNVGKDLLDLSGRLIAQKEDIDNNIN |
| | HIYELAQQQDQHSSDIKTLKNNVEEGLLELSGHLIDQKADLTKDIKTLESNVEEGLLDLSG |
| | RLIDQKADIAQNQANIQDLAAYNELQDQYAQKQTEAIDALNKASSENTQNIEDLAAYNELQ |
| | DAYAKQQTEAIDALNKASSENTQNIAKNQADIANNINNIYELAQQQDQHSSDIKTLAKVSA |
| | ANTDRIAKNKADADASFETLTKNQNTLIEKDKEHDKLITANKTAIDANKASADTKFAATAD |
| | AITKNGNAITKNAKSITDLGTKVDGFDGRVTALDTKVNAFDGRITALDSKVENGMAAQAAL |
| | SGLFQPYSVGKFNATAALGGYGSKSAVAIGAGYRVNPNLAFKAGAAINTSGNKKGSYNIGV |
| | NYEF (614 amino acids) |
| American | MKTMKLPPLKIAVTSAMIIGLGAASTANAQTTETFLPNLFDNDYTETTDPLYHGMILGDTA |
| V1118 | ITQDTQYKFYAENGNEVPDSLFFNKILHDQLLNGFKAGDTIIPLDENGKPVYKLDERTENG |
| (SEQ ID | VKRKVYSVTTKTATQADVEQSAYSRGIQGDIDDLYEANKENVNRLIEHGDKIFANEESVQY |
| NO: 56) | LNREVQNNIENIHELAQQQDQHSSDIKTLKKNVEKDLLDLSGRLIAQKEDIAQNQTDIQDL |
| | ATYNELQDQYAQKQTEAIDALNKASSENTQNIAKNSNHIKTLENNIEECLLELSGHLIDQK |
| | ADLTKDIKALESNVEEGLLDLSGRLIDQKADIAQNQANIQDLAAYNELQDAYAKQQTEAID |
| | ALNKASSENTQNIEDLAAYNELQDAYAKQQTEAIDALNKASSENTQNIEDLAAYNELQDAY |
| | AKQQTEAIDALNKASSENTQNIAKNQADIANNINNIYELAQQQDQHSSDIKTLAKASAANT |
| | DRIAKNKADADASFETLTKNQNTLIEKDKEHDKLITANKTAIDANKASADTKFAATADAIT |
| | KNGNAITKNAKSITDLGTKVDGFDGRVTALDTKVNALDTKVNAFDGRITALDSKVENGMAA |
| | QAALSGLFQPYSVGKFNATAALGGYGSKSAVAIGAGYRVNPNLAFKAGAAINTSGNKKGSY |
| | NIGVNYEF (679 amino acids) |
| American | MKTMKLLPLKIAVTSALIVGLGAASTANAQETLEEVLESIKQINEQDLQDDIGYNSALDRY |
| V1145 | LVLSQYGNLLIAKELNENVEKNSNSIAKNSNSIADLEADVGYLAENQNTLIEQNETINQEL |
| (SEQ ID | EGITHELESFIAYAHAQDQKNLVNEFEIEKNKDAIAKNNESIEDLYDFGHEVAESIGEIHA |
| NO: 57) | YTEEVNKTLENLITNSVKNTDNITKNKADIQALESNVEKELLNLSGRLIDQKADIDNNINH |
| | IYELAQQQDQHSSDIKTLKKNVEEGLLELSGHLIDQKSDIAQNQTDIQDLATYNELQDQYA |
| | QKQTEAIDALNKASSENTQNIEDLAAYNELQDAYAKQQTEAIDALNKASSENTQNIEDLAA |
| | YNELQDAYAKQQTEAIDALNKASSENTQNIEDLAAYNELQDAYAKQQTEAIDALNKASSEN |
| | TQNIEDLAAYNELQDAYAKQQTEAIDALNKASSENTQNIEDLAAYNELQDAYAKQQTEAID |
| | ALNKASSENTQNIAKNQADIANNINNIYELAQQQDQHSSDIKTLAKASAANTDRIAKNKAD |
| | ADASFETLTKNQNTLIEKDKEHDKLITANKTAIDANKASADTKFAATADAITKNGNAITKN |
| | AKSITDLGTKVDGFDSRVTALDTKVNAFDGRITALDSKVENGMAAQAALSGLFQPYSVGKF |
| | NATAALGGYGSKSAVAIGAGYRVNPNLAFKAGAAINTSGNKKGSYNIGVNYEF |
| | (724 amino acids) |
| American | MKTMKLLPLKIAVTSALIVGLGAASTANAQAQARDRSLEDIQALIGNIDVDKIRSQKQKNP |
| V1156 | EIFQYLLLNQLSNTLITDELNNNVIKNTNSIETLDNDIAWLNDDLIDLDKEVGVLSRDIGS |
| (SEQ ID | LHDDVAQNQADIKTLENNVVEELFNLSDRLIDQEAEIAQNNESIEDLYDFGREVAESIGEI |
| NO: 58) | HAHNEAQNETLKDLITNSVKNTDNIDKNKADIQALENNVEEGLLELSGHLIDQKADLTKDI |
| | KALESNVEEGLLDLSGRLLDQKADIAKNQADIAQNQTDIQDLAAYNELQDQYAQKQTEAID |
| | ALNKASSENTQNIEDLAAYNELQDAYAKQQTEAIDALNKASSENTQNIEDLAAYNELQDAY |
| | AKQQTEAIDALNKASSENTQNIAKNQADIANNINNIYELAQQQDQHSSDIKTLAKVSAANT |
| | DRIAKNKADADASFETLTKNQNTLIEKDKEHDKLITANKTAIDANKASADTKFAATADAIT |
| | KNGNAITKNAKSITDLGTKVDGFDSRVTALDTKVNAFDGRITALDSKVENGMAAQAALSGL |
| | FQPYSVGKFNATAALGGYGSKSAVAIGAGYRVNPNLAFKAGAAINTSGNKKGSYNIGVNYE |
| | F (611 amino acids) |

UspA2 may be UspA2 from M. catarrhalis strain ATCC(a US registered trademark) 25238™, American 2933. American 2912, American 2908, Finnish 307, Finnish 353, Finnish 358, Finnish 216, Dutch H2, Dutch F10, Norwegian 1, Norwegian 13, Norwegian 20, Norwegian 25, Norwegian 27, Norwegian 36, BC5SV, Norwegian 14, Norwegian 3, Finish 414, Japanese Z7476, Belgium Z7530, German Z8063, American 012E, Greek MC317, American V1122, American P44, American V1171, American TTA24, American 035E, American SP12-6, American SP12-5, Swedish BC5, American 7169, Finnish FIN2344, American V1118, American V1145 or American V1156. UspA2 may be UspA2 as set forth in any of SEQ ID NO: 10 or SEQ ID NO: 22-SEQ ID NO: 38. UspA2 may be UspA2 from another source which corresponds to the sequence of UspA2 in any one of SEQ ID NO: 10 or SEQ ID NO: 22-SEQ ID NO: 58.

UspA2 may be a sequence with at least 95% identity, over the entire length, to any of SEQ ID NO: 10 or SEQ ID NO: 22-SEQ ID NO: 58. In one embodiment, UspA2 may be a sequence as set forth in an amino acid sequence selected from the group consisting of SEQ ID NO: 10, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57 and SEQ ID NO: 58 or any subset of SEQ ID NO: 1 or SEQ ID NO:22 through SEQ ID NO:58.

Immunogenic fragments of UspA2 comprise immunogenic fragments of at least 450 contiguous amino acids of SEQ ID NO: 1, 490 contiguous amino acids of SEQ ID NO: 10 (for example, the UspA2 fragment of MC-004 or MC-005), 511 contiguous amino acids of SEQ ID NO: 10 (for example, the UspA2 fragment of construct MC-001, MC-002, MC-003 or MC-004), 534 contiguous amino acids of SEQ ID NO: 10 (for example, the UspA2 fragment of MC-009 or MC-011) or 535 contiguous amino acids of SEQ ID NO: 10 (for example, the UspA2 fragment of MC-007, MC-008 or MC-010). The immunogenic fragments may elicit antibodies which can bind SEQ ID NO: 10.

Immunogenic fragments of UspA2 may comprise immunogenic fragments of at least 450, 490, 511, 534 or 535 contiguous amino acids of SEQ ID NO: 10. Immunogenic fragments of UspA2 may comprise immunogenic fragments of UspA2, for example any of the UspA2 constructs MC-001 (SEQ ID NO: 11), MC-002 (SEQ ID NO: 12), MC-003 (SEQ ID NO: 13), MC-004 (SEQ ID NO: 14), MC-005 (SEQ ID NO: 15), MC-006 (SEQ ID NO: 16), MC-007 (SEQ ID NO: 17), MC-008 (SEQ ID NO:18), MC-009 (SEQ ID NO: 19), MC-010 (SEQ ID NO: 20) or MC-011 (SEQ ID NO: 21). The immunogenic fragments may elicit antibodies which can bind the full length sequence from which the fragment is derived.

In another aspect of the present invention, the immunogenic composition comprises an immunogenic fragment of UspA2, suitably an isolated immunogenic polypeptide with at least 70%, 80%, 85%, 90%, 91%, 92%, 93%, 94%, 95%, 96%, 97%, 98%, 99%, or 100% to a polypeptide selected from the group consisting of MC-001 (SEQ ID NO: 11), MC-002 (SEQ ID NO: 12), MC-003 (SEQ ID NO: 13), MC-004 (SEQ ID NO: 14), MC-005 (SEQ ID NO: 15), MC-006 (SEQ ID NO: 16), MC-007 (SEQ ID NO: 17), MC-008 (SEQ ID NO:18), MC-009 (SEQ ID NO: 19), MC-010 (SEQ ID NO: 20) or MC-011 (SEQ ID NO: 21) e.g. MC009 SEQ ID NO: 19 (corresponding to SEQ ID NO: 69 of WO2015/125118A1). For example, an immunogenic polypeptide of UspA2 may comprise (or consist) of the amino acid sequence of SEQ ID NO: 19 (MC009).

Immunogenicity of UspA2 polypeptides may be measured as described in WO2015/125118A1; the contents of which are incorporated herein by reference.

The immunogenic compositions of the present invention may comprise an immunogenic polypeptide of Protein D from non-typeable H. influenzae (NTHi), a PE-PilA fusion protein and an immunogenic polypeptide of UspA2 from M. catarrhalis for example:
PD 10 μg/PE-PilA (LVL735 construct, as described in WO2012/139225) 10 μg/UspA2 (MC009 construct, as described in WO2015125118) 10 μg/ASO1E
PD 10 μg/PE-PilA (LVL735 construct, as described in WO2012/139225) 10 μg/20

UspA2 (MC009 construct, as described in WO2015125118) 3.3 μg/ASO1E The above two specific immunogenic compositions were evaluated in a mouse Moraxella catarrhalis lung inflammation model in WO2015125118 (Example 14).

In one embodiment, identity is calculated using the Needle program, from the EMBOSS package (Free software; EMBOSS: The European Molecular Biology Open Software Suite). In another embodiment, identity is calculated using the algorithm described by Dufresne et al. in Nature Biotechnology in 2002 (vol. 20, pp. 1269-71) and is used in the software GenePAST (Genome Quest Life Sciences, Inc. Boston, M A, 2021).

### Dosage

The amount of the immunogenic composition which is required to achieve the desired therapeutic or biological effect will depend on a number of factors such as means of administration, the recipient and the type and severity of the condition being treated, and will be ultimately at the discretion of the attendant physician or veterinarian. The present invention provides an immunogenic composition comprising an immunogenic polypeptide from non-typeable Haemophilus influenzae or an immunogenic fragment thereof and/or an immunogenic polypeptide from Moraxella catarrhalis or an immunogenic fragment thereof for use in the treatment of chronic obstructive pulmonary disease (COPD) associated with a bacterial infection in a subject. In general, a typical dose of the immunogenic polypeptide from Moraxella catarrhalis or an immunogenic fragment thereof may be expected to lie in the range of from about 0.001 mg-0.120 mg. More specifically, a typical dose in a human may lie in the range of from about 0.003 mg to about 0.03 mg of an immunogenic polypeptide. In general, a typical dose of the immunogenic polypeptide from H. influenzae or an immunogenic fragment thereof may be expected to lie in the range of from about 0.005 mg to about 0.05 mg. This dose may be administered as a single unit dose. Several separate unit doses may also be administered. For example, separate unit doses may be administered as separate priming doses within the first year of life or as separate booster doses given at regular intervals (for example, every 1, 5 or 10 years).

The present invention provides an immunogenic composition comprising: (i) an immunogenic polypeptide from non-typeable Haemophilus influenzae, (ii) a PE-PilA fusion protein, (iii) optionally an immunogenic polypeptide of UspA2 from Moraxella catarrhalis, and (iv) an adjuvant, for use in reducing the frequency of severe exacerbations, in a subject (e.g. human) having chronic obstructive pulmonary disease (COPD) comprising: (a) selecting a subject who has COPD (e.g. GOLD 4 (very severe) COPD), and (b) administering to the subject the immunogenic composition. Suitably, the immunogenic composition is in the format of an intramuscular injection. Suitably, the immunogenic composition is administered to a subject in at least two doses e.g. 2 doses or 3 doses. Thus the present invention also provides an immunogenic composition for use, use of an immunogenic composition or method according to the invention, wherein the immunogenic composition is in the format of an intramuscular injection and is administered to a subject in at least two doses. In a further embodiment, two doses of an immunogenic composition according to the present invention are administered, optionally according to a 0, 2 month vaccination schedule, wherein the second dose is administered about two-months after the first dose (e.g. at the end of the second month or at the beginning or the third month, for example, the first dose on Day 1 and the second dose on Day 61).

### Formulations and Adjuvants

Formulations comprising the immunogenic compositions of the present invention may be adapted for administration by an appropriate route, for example, by the intramuscular, sublingual, transcutaneous, intradermal or intranasal route. In one embodiment, the immunogenic compositions of the present invention are administered intramuscularly. Such formulations may be prepared by any method known in the art.

The immunogenic compositions of the present invention may additionally comprise an adjuvant. When the term “adjuvant” is used in this specification, it refers to a substance that is administered in conjunction with the immunogenic composition to boost the patient's immune response to the immunogenic component of the composition.

Suitable adjuvants include an aluminum salt such as aluminum hydroxide gel or aluminum phosphate or alum, but may also be a salt of calcium, magnesium, iron or zinc, or may be an insoluble suspension of acylated tyrosine, or acylated sugars, cationically or anionically derivatized saccharides, or polyphosphazenes. In one embodiment, the immunogenic polypeptide(s) may be adsorbed onto aluminium phosphate. In another embodiment, the immunogenic polypeptide(s) may be adsorbed onto aluminium hydroxide. In a third embodiment, alum may be used as an adjuvant.

Suitable adjuvant systems which promote a predominantly Th1 response include: non-toxic derivatives of lipid A, Monophosphoryl lipid A (MPL) or a derivative thereof, particularly 3-de-O-acylated monophosphoryl lipid A (3D-MPL) (for its preparation see GB 2220211 A); and a combination of monophosphoryl lipid A, preferably 3-de-O-acylated monophosphoryl lipid A, together with either an aluminum salt (for instance aluminum phosphate or aluminum hydroxide) or an oil-in-water emulsion. In such combinations, antigen and 3D-MPL are contained in the same particulate structures, allowing for more efficient delivery of antigenic and immunostimulatory signals. Studies have shown that 3D-MPL is able to further enhance the immunogenicity of an alum-adsorbed antigen (Thoelen et al. Vaccine (1998) 16:708-14; EP 689454-B1).

AS01 is an Adjuvant System containing MPL (3-O-desacyl-4′-monophosphoryl lipid A), QS21 ((Quillaja saponaria Molina, fraction 21) Antigenics, New York, NY, USA) and liposomes. AS01B is an Adjuvant System containing MPL, QS21 and liposomes (50 μg MPL and 50 μg QS21). ASO1E is an Adjuvant System containing MPL, QS21 and liposomes (25 μg MPL and 25 μg QS21). In one embodiment, the immunogenic composition or vaccine comprises AS01. In another embodiment, the immunogenic composition or vaccine comprises AS01B or ASO1E. In a particular embodiment, the immunogenic composition or vaccine comprises ASO1E.

AS02 is an Adjuvant Aystem containing MPL and QS21 in an oil/water emulsion. ASO2V is an Adjuvant System containing MPL and QS21 in an oil/water emulsion (50 μg MPL and 50 μg QS21).

AS03 is an Adjuvant System containing α-Tocopherol and squalene in an oil/water (o/w) emulsion. ASO3_{A} is an Adjuvant System containing α-Tocopherol and squalene in an o/w emulsion (11.86 mg tocopherol). ASO3_{B} is an Adjuvant System containing α-Tocopherol and squalene in an o/w emulsion (5.93 mg tocopherol). AS03_{C} is an Adjuvant System containing α-Tocopherol and squalene in an o/w emulsion (2.97 mg tocopherol). In one embodiment, the immunogenic composition or vaccine comprises AS03.

AS04 is an Adjuvant System containing MPL (50 μg MPL) adsorbed on an aluminum salt (500 μg Al³⁺). In one embodiment, the immunogenic composition or vaccine comprises AS04.

A system involving the use of QS21 and 3D-MPL is disclosed in WO 94/00153. A composition wherein the QS21 is quenched with cholesterol is disclosed in WO 96/33739. An additional adjuvant formulation involving QS21, 3D-MPL and tocopherol in an oil in water emulsion is described in WO 95/17210. In one embodiment the immunogenic composition additionally comprises a saponin, which may be QS21. The formulation may also comprise an oil in water emulsion and tocopherol (WO 95/17210). Unmethylated CpG containing oligonucleotides (WO 96/02555) and other immunomodulatory oligonucleotides (WO 0226757 and WO 03507822) are also preferential inducers of a TH1 response and are suitable for use in the present invention.

Additional adjuvants are those selected from the group of metal salts, oil in water emulsions, Toll like receptor agonists, (in particular Toll like receptor 2 agonist, Toll like receptor 3 agonist, Toll like receptor 4 agonist, Toll like receptor 7 agonist, Toll like receptor 8 agonist and Toll like receptor 9 agonist), saponins or combinations thereof.

Possible excipients include arginine, pluronic acid and/or polysorbate. In a preferred embodiment, polysorbate 80 (for example, TWEEN (a US registered trademark) 80) is used. In a further embodiment, a final concentration of about 0.03% to about 0.06% is used. Specifically, a final concentration of about 0.03%, 0.04%, 0.05% or 0.06% polysorbate 80 (w/v) may be used.

Thus, in one aspect of the present invention, the immunogenic composition comprises a pharmaceutically acceptable excipient or carrier.

In another aspect of the present invention, the immunogenic composition comprises an adjuvant, e.g. ASO1E.

### Seasonality

The present invention provides the immunogenic composition for use, use of an immunogenic composition or method according to the invention for reducing the rate of severe exacerbations (and related hospitalizations) at any time during the year (i.e. independent of season). The present invention also provides the immunogenic composition for use, use of an immunogenic composition or method according to the invention for reducing the rate of severe exacerbations (and related hospitalizations) during the winter season (e.g. December to February in Europe/US). The present invention also provides the immunogenic composition for use, use of an immunogenic composition or method according to the invention for reducing the rate of severe exacerbations (and related hospitalizations) during the spring season (e.g. March to May in Europe/US). The present invention also provides the immunogenic composition for use, use of an immunogenic composition or method according to the invention for reducing the rate of severe exacerbations (and related hospitalizations) during the summer season (e.g. June to August in Europe/US). The present invention also provides the immunogenic composition for use, use of an immunogenic composition or method according to the invention for reducing the rate of severe exacerbations (and related hospitalizations) during the autumn season (e.g. September to November in Europe/US).

### Embodiments of the Invention are Further Described in the Subsequent Numbered Paragraphs:

1. An immunogenic composition comprising: (i) an immunogenic polypeptide from non-typeable Haemophilus influenzae, (ii) a PE-PilA fusion protein, (iii) optionally an immunogenic polypeptide of UspA2 from Moraxella catarrhalis, and (iv) an adjuvant, for use in reducing the frequency of severe exacerbations, in a subject (e.g. human) having chronic obstructive pulmonary disease (COPD).
2. The use of an immunogenic composition comprising: (i) an immunogenic polypeptide from non-typeable Haemophilus influenzae, (ii) a PE-PilA fusion protein, (iii) optionally an immunogenic polypeptide of UspA2 from Moraxella catarrhalis, and (iv) an adjuvant, in the manufacture of a medicament for reducing the frequency of severe exacerbations, in a subject (e.g. human) having chronic obstructive pulmonary disease (COPD).
3. A method of reducing the frequency of severe exacerbations, in a subject (e.g. human) having chronic obstructive pulmonary disease (COPD), said method comprising administering to said subject a therapeutically effective amount of an immunogenic composition comprising: (i) an immunogenic polypeptide from non-typeable Haemophilus influenzae, (ii) a PE-PilA fusion protein, (iii) optionally an immunogenic polypeptide of UspA2 from Moraxella catarrhalis, and (iv) an adjuvant.
4. The immunogenic composition for use, use of an immunogenic composition or method according to paragraphs 1-3, for reducing the yearly rate of severe exacerbations, compared to a subject who has not been administered the immunogenic composition.
5. The immunogenic composition for use, use of an immunogenic composition or method according to paragraphs 1-4, for reducing the likelihood of hospitalization due to an AECOPD in a subject (e.g. human) having chronic obstructive pulmonary disease (COPD).
6. The immunogenic composition for use, use of an immunogenic composition or method according to paragraphs 1-5, for reducing the frequency of pneumonia due to COPD (e.g. due to an AECOPD) in a subject (e.g. human) having chronic obstructive pulmonary disease (COPD).
7. The immunogenic composition for use, use of an immunogenic composition or method according to paragraphs 1-6, for reducing the need for intensive care and/or mechanical ventilation due to COPD (e.g. due to an AECOPD) in a subject having chronic obstructive pulmonary disease (COPD).
8. The immunogenic composition for use, use of an immunogenic composition or method according to paragraphs 1-7, for reducing the likelihood of mortality due to an AECOPD in a subject having COPD.
9. The immunogenic composition for use, use of an immunogenic composition or method according to paragraphs 1-8, wherein the subject has an FEV₁<30% predicted.
10. The immunogenic composition for use, use of an immunogenic composition or method according to paragraphs 1-9, wherein the subject has GOLD 4 (very severe) COPD status.
11. The immunogenic composition for use, use of an immunogenic composition or method according to paragraphs 1-10, wherein the subject has GOLD 4 (very severe) COPD status for reducing the yearly rate of severe exacerbations (e.g. by at least 19%) compared to a subject who has not been administered the immunogenic composition.
12. The immunogenic composition for use, use of an immunogenic composition or method according to paragraphs 1-11 for reducing the frequency of severe exacerbations, in a subject having COPD comprising: (a) selecting a subject who has GOLD 4 (very severe) COPD status, and (b) administering to the subject the immunogenic composition.
13. The immunogenic composition for use, use of an immunogenic composition or method according to paragraphs 1-12, wherein the subject has experienced at least 2 moderate AECOPD or at least one severe AECOPD in the previous 12 months.
14. The immunogenic composition for use, use of an immunogenic composition or method according to paragraphs 1-13, wherein the subject is taking ICS.
15. The immunogenic composition for use, use of an immunogenic composition or method according to paragraphs 1-14, wherein the subject has experienced at least 2 moderate AECOPD or at least one severe AECOPD in the previous 12 months and is taking ICS.
16. The immunogenic composition for use, use of an immunogenic composition or method according to paragraphs 1-15, wherein the subject has experienced at least 2 moderate AECOPD or at least one severe AECOPD in the previous 12 months and is taking short term and long term bronchodilators and ICS.
17. The immunogenic composition for use, use of an immunogenic composition or method according to paragraphs 1-16, wherein the subject has experienced at least 2 moderate AECOPD or at least one severe AECOPD in the previous 12 months for reducing the yearly rate of severe exacerbations (e.g. by at least 13%) compared to a subject who is not administered the immunogenic composition.
18. The immunogenic composition for use, use of an immunogenic composition or method according to paragraphs 1-17 for reducing the frequency of severe exacerbations, in a subject having COPD comprising: (a) selecting a subject who has COPD that is partially controlled or uncontrolled by ICS and has experienced at least 2 moderate AECOPD or at least one severe AECOPD in the previous 12 months, and (b) administering to the subject the immunogenic composition.
19. The immunogenic composition for use, use of an immunogenic composition or method according to paragraphs 1-18 wherein the subject has previously been administered a pneumococcal vaccine.
20. The immunogenic composition for use, use of an immunogenic composition or method according to paragraphs 1-19 wherein the subject has been administered an influenza vaccine in previous 12 months.
21. The immunogenic composition for use, use of an immunogenic composition or method according to paragraphs 1-20 wherein the subject has previously been administered a pneumococcal vaccine and/or has been administered an influenza vaccine in the previous 12 months.
22. The immunogenic composition for use, use of an immunogenic composition or method according to paragraphs 1-21, wherein the subject has experienced at least one severe AECOPD in the previous 12 months.
23. The immunogenic composition for use, use of an immunogenic composition or method according to paragraphs 1-22 for reducing the rate of severe exacerbations (and related hospitalizations) during the winter season.
24. The immunogenic composition for use, use of an immunogenic composition or method according to paragraphs 1-23 for reducing the rate of severe exacerbations (and related hospitalizations) during the spring season.
25. The immunogenic composition for use, use of an immunogenic composition or method according to paragraphs 1-24 for reducing the rate of severe exacerbations (and related hospitalizations) during the summer season.
26. The immunogenic composition for use, use of an immunogenic composition or method according to paragraphs 1-25 for reducing the rate of severe exacerbations (and related hospitalizations) during the autumn season.
27. The immunogenic composition for use, use of an immunogenic composition or method according to any of paragraphs 1 to 26, wherein the immunogenic polypeptide from non-typeable Haemophilus influenzae is an immunogenic polypeptide of Protein D, suitably an isolated immunogenic polypeptide with at least 70%, 80%, 85%, 90%, 91%, 92%, 93%, 94%, 95%, 96%, 97%, 98%, 99%, or 100% to SEQ ID NO: 2.
28. The immunogenic composition for use, use of an immunogenic composition or method according to paragraphs 1 to 27, wherein the PE-PilA fusion protein is an isolated immunogenic polypeptide with at least 70%, 80%, 85%, 90%, 91%, 92%, 93%, 94%, 95%, 96%, 97%, 98%, 99%, or 100% to SEQ ID NO: 9.
29. The immunogenic composition for use, use of an immunogenic composition or method according to paragraphs 1-28, wherein the composition comprises an immunogenic polypeptide of UspA2 from Moraxella catarrhalis, suitably an isolated immunogenic polypeptide with at least 70%, 80%, 85%, 90%, 91%, 92%, 93%, 94%, 95%, 96%, 97%, 98%, 99%, or 100% to a polypeptide selected from the group consisting of MC-001 (SEQ ID NO: 11), MC-002 (SEQ ID NO: 12), MC-003 (SEQ ID NO: 13), MC-004 (SEQ ID NO: 14), MC-005 (SEQ ID NO: 15), MC-006 (SEQ ID NO: 16), MC-007 (SEQ ID NO: 17), MC-008 (SEQ ID NO:18), MC-009 (SEQ ID NO: 19), MC-010 (SEQ ID NO: 20) or MC-011 (SEQ ID NO: 21) e.g. MC009 SEQ ID NO: 19.
30. The immunogenic composition for use, use of an immunogenic composition or method according to any of paragraphs 1 to 29, wherein the immunogenic composition comprises a pharmaceutically acceptable excipient or carrier.
31. The immunogenic composition for use, use of an immunogenic composition or method according to any of paragraphs 1 to 30, wherein the adjuvant is an AS01 adjuvant, e.g. ASO1E.
32. The immunogenic composition for use, use of an immunogenic composition or method according to any of paragraphs 1 to 31, wherein the immunogenic composition is in the format of an intramuscular injection and is administered to a subject in at least two doses.

### EXAMPLES

### Example 1—Phase IIB Study of Investigational NTHi-Mcat Vaccine

An AS01E-adjuvanted formulation containing 10 μg of PD, 10 μg of the PE-PilA fusion protein and 3.3 μg of UspA2 was evaluated in a Phase IIB study. The antigens and formulation were prepared and tested as described in WO2015/125118.

TABLE 4
Study vaccines

| Treatment | Vaccine/ | | | Volume to be | Number |
|---|---|---|---|---|---|
| name | product name | Formulation | Presentation | administered | of doses |
| 10-10-3/ | NTHi-Mcat/ | PD = 10 μg; PE-PilA = 10 μg; | Freeze-dried | 0.5 ml | 2 |
| AS01_{E} | 10-10-3.3 | UspA2 = 3.3 μg | antigens in | | |
| | | | monodose vial | | |
| | AS01E | MPL = 25 μg; QS21 = 25 μg; | Liquid in | | |
| | | Liposomes | monodose vial | | |
| Placebo | Formulation | Na₂HPO₄ = 0.4 mg; | Liquid in | 0.5 ml | 2 |
| | buffer S9b | KH₂PO₄ = 56 μg; | monodose vial | | |
| | | NaCl = 1.16 mg; KCl = 30 μg; | | | |
| | | MgCl₂ = 15 μg | | | |

MPL = 3-O-desacyl-4′-monophosphoryl lipid A; QS-21 = Quillaja saponaria Molina, fraction 21 (Licensed by GSK from Antigenics Inc, a wholly owned subsidiary of Agenus Inc., a Delaware, USA corporation).

Experimental Design

A Phase IIB, randomised, observer-blind, placebo-controlled, multi-centre study was carried out to evaluate the efficacy, safety, reactogenicity and immunogenicity of the vaccine when administered intramuscularly according to a 0, 2 month schedule in COPD patients aged 40 to 80 years with a previous history of acute exacerbation (AECOPD).

In this study, moderate, severe and very severe COPD patients (i.e. GOLD grade 2, 3 and 4) with a history of AECOPD received 2 doses of the NTHi-Mcat investigational vaccine or placebo intramuscularly (IM) according to a 0, 2 month vaccination schedule, in addition to standard care.

Study Groups

Eligibility criteria included patients aged 40-80 years with a documented history of at least one moderate or severe AECOPD in the previous 12 months. COPD was confirmed as moderate, severe, or very severe, according to Global Initiative for Chronic Obstructive Lung Disease (GOLD) grade 2, 3, or 4, based on post-bronchodilator spirometry, with forced expiratory volume in 1 second over forced vital capacity ratio (FEVs/FVC)<0.7, and FEV₁<80% of predicted normal. A documented history of AECOPD was defined as a medical record of worsening COPD symptoms that required systemic or oral corticosteroids and/or antibiotics (moderate exacerbation) or hospitalization (severe exacerbation).

Patients received intramuscular injections of two doses of the candidate NTHi-Mcat vaccine or placebo (phosphate-buffered saline). The NTHi-Mcat vaccine contained 10 μg PD, 10 μg PE-PilA, and 3.3 μg UspA2 and included the Adjuvant System AS01, a liposome-based vaccine adjuvant system containing two immunostimulants (3-O-desacyl-4′-monophosphoryl lipid A and the saponin QS-21 with adjuvantation).

TABLE 5
Dosage and administration

| Type of | | | Volume to | | | | |
|---|---|---|---|---|---|---|---|
| contact and | Study | Treatment | be | | Site | | |
| timepoint | group | name | administered | Route ¹ | Location | Directionality² | Laterality³ |
| Visit 1 | 10-10-3-AS | 10-10-3/AS01E | 0.5 ml | IM | Deltoid | Upper | Non- |
| (Day 1) | CONTROL | Placebo | | | | | dominant |
| Visit 3 | 10-10-3-AS | 10-10-3/AS01E | 0.5 ml | IM | Deltoid | Upper | Non- |
| (Day 61) | CONTROL | Placebo | | | | | dominant |

¹ Intramuscular (IM)
²Directionality is a qualifier for further detailing the location of the vaccine administration (e.g. Upper, Lower)
³The non-dominant arm is the preferred arm of injection. In case it is not possible to administer the vaccine in the non-dominant arm, an injection in the dominant arm may be performed.

All Subjects were Asked to Record COPD Symptoms in their Electronic Diary Card:
Daily in the morning throughout the study (including during AECOPD): morning symptoms questionnaire.
Daily in the evening throughout the study (including during AECOPD): EXACT-PRO questionnaire

A potential exacerbation was defined, based on Anthonisen criteria (Anthonisen N R, Manfreda J, Warren C P, Hershfield E S, Harding G K, Nelson N A. Antibiotic therapy in exacerbations of chronic obstructive pulmonary disease. Ann Intern Med 1987; 106: 196-204), as worsening of two or more of the major symptoms, dyspnoea, sputum volume, and sputum purulence, for at least two consecutive days, or worsening of any major symptom together with any of the following minor symptoms for at least two consecutive days: sore throat, cold (nasal discharge and/or nasal congestion), fever (oral temperature ≥37.5° C.), increased cough, and increased wheeze.

Vaccine efficacy was defined as (1-(R_{Vaccine}/R_{Placebo}))*100, where R_{vaccine} is the average yearly rate of (moderate and) severe AECOPD per patient in the vaccine group and R_{placebo} is the average yearly rate of (moderate and) severe AECOPD per patient in the placebo group.

Results

Between Nov. 27, 2017, and Nov. 30, 2018, 606 adults with confirmed COPD and a history of moderate/severe AECOPD were recruited and allocated, through a randomisation algorithm, to receive two doses of the NTHi-Mcat vaccine or placebo. The study was completed by 281 patients in the vaccine group and 263 in the placebo group, with 23 and 39 dropouts, respectively; four and 15, respectively, withdrew because of an AE. The primary analysis population (modified total vaccinated cohort) included 571 patients who received two doses (279 in NTHi-Mcat vaccine group, 292 in placebo group). Patients' baseline characteristics were well balanced between groups. In the primary analysis population, 271 (47.5%) patients had a history of one moderate/severe exacerbation and 300 (52.5%) had a history of at least two moderate/severe exacerbations in the year before enrolment.

In the total vaccinated cohort, 1005 exacerbations with known severity were reported during the entire study period (512 in NTHi-Mcat vaccine group and 493 in placebo group). Most exacerbations were moderate (408 in vaccine group, 364 in placebo group). In the vaccine group, 54 mild and 50 severe exacerbations were reported, and 50 mild and 79 severe exacerbations were reported in the placebo group.

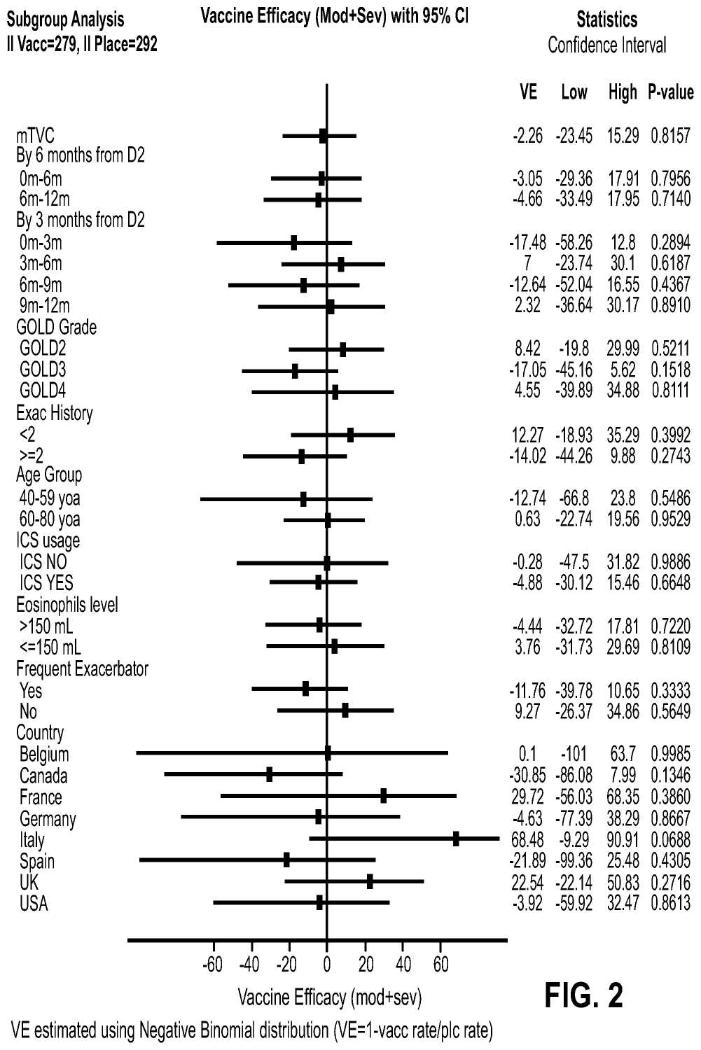

Vaccine efficacy estimates for mild, moderate, and severe exacerbation severity are shown in Table 6. The point estimate for vaccine efficacy in reducing severe episodes was 36.5% (95% CI-4.7 to 61.5; p=0.075). The hazard ratio for relative risk of having a severe AECOPD was 0.72 (95% CI 0.45 to 1.16) for the NTHi-Mcat vaccine group (p=0.176). Results of additional subgroup analyses of vaccine efficacy are presented in FIG. 2. Vaccine efficacy was similar among the subgroups categorised by GOLD grade, country, history of exacerbations, usage of inhaled corticosteroid at the first study visit, eosinophil level at baseline, or age group. Analysis of moderate/severe AECOPD yearly rates derived from consecutive three-month follow-up periods showed no trend for rate reduction (Table 7). This subperiod analysis of AECOPD categorised as moderate or severe showed a trend for reduction in the yearly rate of severe episodes at 9-12 months after one month post-dose 2 (vaccine efficacy 75.8%; 95% CI 27 to 92).

During the 30-day post-vaccination period, at least one unsolicited AE was reported by 36.2% of patients (174 events) in the NTHi-Mcat vaccine group and 34.1% of patients (182 events) in the placebo group, most commonly nasopharyngitis (all mild or moderate and unrelated to vaccination), reported by 4.3% of patients in each group (13 reports in vaccine group, 15 in placebo group). Most of the unsolicited AEs were mild or moderate, with 13 patients (4.3%) in the NTHi-Mcat vaccine group and 19 (6.3%) in the placebo group reporting severe symptoms. Unsolicited AEs considered related to study vaccination were reported by nine patients (3.0%; 16 events) in the NTHi-Mcat vaccine group and eight (2.6%; nine events) in the placebo group. There were 149 unsolicited AEs leading to hospitalization reported by 86 patients (28.3%) in the vaccine group and 209 reported by 96 patients (31.8%) in the placebo group. The most common AEs leading to hospitalization were pneumonia (five events in vaccine group, 17 in placebo group) and COPD (55 and 82, respectively).

During the entire study, there was one death in the NTHi-Mcat group (due to acute respiratory failure; not considered related to study vaccination) and 10 in the placebo group (three deaths due to COPD and three to respiratory failure; remainder due to different separate causes). There were 158 serious AEs (SAEs) reported by 89 patients (29.3%) in the NTHi-Mcat vaccine group and 214 SAEs reported by 99 patients (32.8%) in the placebo group, most frequently pneumonia (five reports in the NTHi-Mcat vaccine group, 17 in placebo group), acute respiratory failure (eight and seven reports, respectively), and COPD (55 and 83 reports, respectively). Potential immune-mediated disease was reported for nine patients (six with 10 events in NTHi-Mcat vaccine group, three with three events in placebo group). None of the SAE or potential immune-mediated disease reports were considered related to vaccination.

### CONCLUSION

In conclusion, the phase 2 trial did not meet its primary efficacy objective of reducing the frequency of moderate and severe exacerbations when the NTHi-Mcat vaccine was administered in a two-dose schedule to patients with COPD. The results confirm the vaccine's acceptable safety and reactogenicity profile and good immunogenicity. Observations suggesting possible reductions in the vaccinated group in the frequency (yearly rate) of severe exacerbations and related hospitalizations encourage further evaluation.

TABLE 6
Vaccine efficacy against AECOPD by exacerbation severity

| | | | | Total | | | VE | |
|---|---|---|---|---|---|---|---|---|
| | | N | n | Time | Yearly | | 95% | p- |
| Severity | Group | (Subj) | (events) | (days) | rate | VE | CI | value |
| MILD | 10-10-3 | 278 | 37 | 102123 | 0.13 | −9.48% | −94%:38% | 0.756 |
| | AS01E | | | | | | | |
| | Placebo | 292 | 33 | 104443 | 0.12 | | | |
| MOD | 10-10-3 | 278 | 302 | 102123 | 1.08 | −10.2% | −35%:10% | 0.3561 |
| | AS01E | | | | | | | |
| | Placebo | 292 | 274 | 104443 | 0.96 | | | |
| SEVERE | 10-10-3 | 278 | 38 | 102123 | 0.14 | 36.54 | −4.7%:62% | 0.075 |
| | AS01E | | | | | | | |
| | Placebo | 292 | 59 | 104443 | 0.21 | | | |

TABLE 7
Yearly rate of AECOPD by three-month subperiod, according to exacerbation severity

| | | | | | | | |
|---|---|---|---|---|---|---|---|
| Period | | | | Total | Yearly | Vaccine | |
| (relative to | | Number | | follow- | rate | efficacy | |
| 1 month | | of | Number of | up time | (95% | (95% | p |
| post-dose 2) | Group | patients | exacerbations | (days) | CI) | CI) | value |

Moderate and severe AECOPD

| | | | | | | | |
|---|---|---|---|---|---|---|---|
| Before | NTHi- | 279 | 90 | 26898 | 1.22 | 2.51% | 0.8622 |
| | Mcat | | | | (0.99 | (−29 94 | |
| | vaccine | | | | to 1.50) | to 26 86) | |
| | Placebo | 292 | 97 | 28120 | 1.26 | | |
| | | | | | (1.03 | | |
| | | | | | to 1.54) | | |
| 0-3 | NTHi- | 278 | 92 | 24810 | 1.35 | −17.48% | 0.2894 |
| months | Mcat | | | | (1.10 | (−58.26 | |
| after | vaccine | | | | to 1.66) | to 12.80) | |
| | Placebo | 292 | 82 | 26066 | 1.15 | | |
| | | | | | (0.92 | | |
| | | | | | to 1.43) | | |
| 3-6 | NTHi- | 274 | 89 | 24507 | 1.33 | 7.00% | 0.6187 |
| months | Mcat | | | | (1.08 | (−23.74 | |
| after | vaccine | | | | to 1.63) | to 30.10) | |
| | Placebo | 288 | 101 | 25550 | 1.44 | | |
| | | | | | (1.19 | | |
| | | | | | to 1.75) | | |
| 6-9 | NTHi- | 272 | 91 | 24416 | 1.36 | −12.64% | 0.4367 |
| months | Mcat | | | | (1.11 | (−52.04 | |
| after | vaccine | | | | to 1.67) | to 16.55) | |
| | Placebo | 280 | 81 | 24873 | 1.19 | | |
| | | | | | (0.96 | | |
| | | | | | to 1.48) | | |
| 9-12 | NTHi- | 271 | 68 | 28390 | 0.87 | 2.32% | 0.8910 |
| months | Mcat | | | | (0.69 | (−36.64 | |
| after | vaccine | | | | to 1.11) | to 30.17) | |
| | Placebo | 272 | 69 | 27954 | 0.90 | | |
| | | | | | (0.71 | | |
| | | | | | to 1.14) | | |

Moderate AECOPD

| | | | | | | | |
|---|---|---|---|---|---|---|---|
| 0-3 | NTHi- | 278 | 82 | 24810 | 1.21 | −26.16% | 0.1570 |
| months | Mcat | | | | | (−74 to 9) | |
| after | vaccine | | | | | | |
| | Placebo | 292 | 68 | 26066 | 0.95 | | |
| 3-6 | NTHi- | 274 | 75 | 24507 | 1.12 | 5.17% | 0.7388 |
| months | Mcat | | | | | (−30 to 31) | |
| after | vaccine | | | | | | |
| | Placebo | 288 | 84 | 25550 | 1.20 | | |
| 6-9 | NTHi- | 272 | 81 | 24416 | 1.21 | −17.15% | 0.3346 |
| months | Mcat | | | | | (−62 to 15) | |
| after | vaccine | | | | | | |
| | Placebo | 280 | 69 | 24873 | 1.01 | | |
| 9-12 | NTHi- | 271 | 64 | 28390 | 0.82 | −19.59% | 0.3362 |
| months | Mcat | | | | | (−72 to 17) | |
| after | vaccine | | | | | | |
| | Placebo | 272 | 53 | 27954 | 0.69 | | |

Severe AECOPD

| | | | | | | | |
|---|---|---|---|---|---|---|---|
| 0-3 | NTHi- | 278 | 10 | 24810 | 0.15 | 25.23% | 0.4832 |
| months | Mcat | | | | | (−69 to 67) | |
| after | vaccine | | | | | | |
| | Placebo | 292 | 14 | 26066 | 0.20 | | |
| 3-6 | NTHi- | 274 | 14 | 24507 | 0.21 | 17.82% | 0.5885 |
| months | Mcat | | | | | (−67 to 60) | |
| after | vaccine | | | | | | |
| | Placebo | 288 | 17 | 25550 | 0.24 | | |
| 6-9 | NTHi- | 272 | 10 | 24416 | 0.15 | 14.71% | 0.7114 |
| months | Mcat | | | | | (−98 to 63) | |
| after | vaccine | | | | | | |
| | Placebo | 280 | 12 | 24873 | 0.18 | | |
| 9-12 | NTHi- | 271 | 4 | 28390 | 0.05 | 75.82% | 0.0116 |
| months | Mcat | | | | | (27 to 92) | |
| after | vaccine | | | | | | |
| | Placebo | 272 | 16 | 27954 | 0.21 | | |

95% CI, 95% confidence interval. NTHi, non-typeable Haemophilus influenzae, Mcat, Moraxella catarrhalis
Yearly rate is derived fitting a simple Negative Binomial model without covariates. Vaccine efficacy is estimate using Negative Binomial model with group, country, GOLD grade, history of exacerbation, and age category as covariates and log time as offset variable.

TABLE 8
Vaccine efficacy by “frequent exacerbators” and by AECOPD severity

| | | | | Total | | | VE | |
|---|---|---|---|---|---|---|---|---|
| | | N | n | Time | Yearly | | 95% | p- |
| Severity | Group | (Subjcs) | (events) | (days) | rate | VE | CI | value |
| MILD | 10-10-3 | 164 | 25 | 59539 | 0.15 | −48.6% | −208%:29% | 0.2888 |
| | AS01E | | | | | | | |
| | Placebo | 177 | 16 | 63391 | 0.09 | | | |
| MODER | 10-10-3 | 164 | 208 | 59539 | 1.28 | −30.6% | −67%:−2% | 0.0339 |
| | AS01E | | | | | | | |
| | Placebo | 177 | 170 | 63391 | 0.98 | | | |
| SEVERE | 10-10-3 | 164 | 25 | 59539 | 0.15 | 52.1% | 13%:74% | 0.0153 |
| | AS01E | | | | | | | |
| | Placebo | 177 | 53 | 63391 | 0.31 | | | |

Results: Data presented in Table 8 suggest a reduction in the rate of severe AECOPD (unadjusted p-val=0.0153) and at same time an increase in the rate of moderate AECOPD (unadjusted p-avlue=0.0339) leading to the conclusion that in the vaccinated group there is a reduction in AECOPD severity (AECOPD shift from severe to moderate).

TABLE 9(a)
Vaccine efficacy against AECOPD in freq exacerbators on ICS

| | | | | Yearly rate | | |
|---|---|---|---|---|---|---|
| | | | | (model | VE (model | |
| | Vaccine/ | | | without 4 | with 4 | P- |
| Subgroup | Placebo | N | Event | covariables) | covariables) | value |
| FRQEXA(Y) | 10-10-3 | 130 | 12 | 0.093 | 74.99% | 7.24e−05 |
| ICSBLFL(Y) | AS01E | | | | | |
| | Placebo | 146 | 51 | 0.362 | | |
| All except | 10-10-3 | 149 | 26 | 0.174 | −143.39% | 0.047 |
| FRQEXA(Y) | AS01E | | | | | |
| ICSBLFL(Y) | Placebo | 146 | 8 | 0.057 | | |

Results: using FRQEXA(Y)+ICSBLFL(Y) as biomarkers can identify a subpopulation of COPD patients that has double (75%) vaccine efficacy of severe AECOPD with respect to the non-stratified population (36%). Approximately half of the COPD patients belongs to this subpopulation and they can benefits most from the vaccine.

TABLE 9(b)
Vaccine efficacy against AECOPD in patients on ICS

| | | | | Yearly rate | | |
|---|---|---|---|---|---|---|
| | | | | (model | VE (model | |
| | Vaccine/ | | | without 4 | with 4 | |
| Subgroup | Placebo | N | Event | covariables) | covariables) | P-val |
| ICSBLFL(Y) | 10-10-3 | 203 | 20 | 0.098 | 61.41% | 0.0014 |
| | AS01E | | | | | |
| | Placebo | 225 | 55 | 0.252 | | |

Results: Using ICSBLFL or FRQEXA as single markers can identify subgroups of COPD patients who benefit from vaccine (VE=61.41% for ICSBLFL(Y) subgroup, VE=52.10°/o for FRQEXA(Y) subgroup), the vaccine efficacy of ICSBLFL(Y) subgroup is higher than that of FRQEXA(Y) subgroup. The COPD patients in the subgroup identified by the combination of these 2 markers ICSBLFL(Y)+FRQEXA(Y) has the highest vaccine efficacy (VE=74.99%).

TABLE 10
Vaccine efficacy in GOLD 4 grade and AECOPD severity

| | | | | Total | | | VE | |
|---|---|---|---|---|---|---|---|---|
| GOLD | | N | n | Time | Yearly | | 95% | p- |
| grade 4 | Group | (Subjcs) | (events) | (days) | rate | VE | CI | value |
| MILD | 10-10-3 | 42 | 1 | 15075 | 0.02 | 82.5% | −155%:99% | 0.2024 |
| | AS01E | | | | | | | |
| | Placebo | 41 | 3 | 14431 | 0.08 | | | |
| MODE | 10-10-3 | 42 | 45 | 15075 | 1.11 | −29.3% | −120%:24% | 0.3433 |
| | AS01E | | | | | | | |
| | Placebo | 41 | 38 | 14431 | 0.96 | | | |
| SEVERE | 10-10-3 | 42 | 8 | 15075 | 0.19 | 65.4% | 19%:85% | 0.0148 |
| | AS01E | | | | | | | |
| | Placebo | 41 | 21 | 14431 | 0.53 | | | |

Results: In GOLD 4 patients vaccine efficacy in reducing severe AECOPDs was shown to be more pronounced (i.e. VE=65%, unadjusted p-value=0.0148).

TABLE 11
Vaccine efficacy in a subset of suject who received the influenza
vaccine before COPD/Placebo vaccine and by AECOPD severity

| | | | | Total | | | VE | |
|---|---|---|---|---|---|---|---|---|
| | | N | n | Time | Yearly | | 95% | p- |
| Severity | Group | (Subjcs) | (events) | (days) | rate | VE | CI | value |
| MILD | 10-10-3 | 187 | 29 | 69099 | 0.15 | −5.32% | −98%:44% | 0.8718 |
| | AS01E | | | | | | | |
| | Placebo | 192 | 27 | 67949 | 0.15 | | | |
| MODER | 10-10-3 | 187 | 210 | 69099 | 1.11 | −10.22% | −40%:13% | 0.4293 |
| | AS01E | | | | | | | |
| | Placebo | 192 | 186 | 67949 | 1.00 | | | |
| SEVERE | 10-10-3 | 187 | 26 | 69099 | 0.14 | 39.53% | 0.6%:63% | 0.0473 |
| | AS01E | | | | | | | |
| | Placebo | 192 | 41 | 67949 | 0.23 | | | |

Results: In the subset of COPD patients who were vaccinated with an influenza vaccine, the COPD vaccine showed similar results as in the overall populations (i.e., including subject who did not receive the influenza vaccine previously).

TABLE 12
Vaccine efficacy in a subset of subjects who received the pneumococcal
vaccine before COPD/Placebo vaccine and by AECOPD severity

| | | | | Total | | | VE | |
|---|---|---|---|---|---|---|---|---|
| | | N | n | Time | Yearly | | 95% | p- |
| Severity | Group | (Subjcs) | (events) | (days) | rate | VE | CI | value |
| MILD | 10-10-3 | 141 | 21 | 51960 | 0.15 | −9.37% | −134%:49% | 0.8169 |
| | AS01E | | | | | | | |
| | Placebo | 159 | 20 | 56137 | 0.13 | | | |
| MODER | 10-10-3 | 141 | 171 | 51960 | 1.20 | −18.51% | −54%:8.7% | 0.2029 |
| | AS01E | | | | | | | |
| | Placebo | 159 | 153 | 56137 | 1.00 | | | |
| SEVERE | 10-10-3 | 141 | 18 | 51960 | 0.13 | 52.51% | 2%:77% | 0.0439 |
| | AS01E | | | | | | | |
| | Placebo | 159 | 37 | 56137 | 0.25 | | | |

Results: In the subset of COPD patients who were vaccinated for pneumococcal, the COPD vaccine showed similar results are in the overall populations (that includes subjects who did not receive the pneumoccocal vaccine).

SEQUENCES:
SEQ ID NO: 1:
Protein D (364 amino acids)
MetLysLeuLysThrLeuAlaLeuSerLeuLeuAlaAlaGlyVal
LeuAlaGlyCysSerSerHisSerSerAsnMetAlaAsnThrGIn
MetLysSerAspLysIleIleIleAlaHisArgGlyAlaSerGly
TyrLeuProGluHisThrLeuGluSerLysAlaLeuAlaPheAla
GInGInAlaAspTyrLeuGluGInAspLeuAlaMetThrLysAsp
GlyArgLeuValValIleHisAspHisPheLeuAspGlyLeuThr
AspValAlaLysLysPheProHisArgHisArgLysAspGlyArg
TyrTyrValIleAspPheThrLeuLysGluIleGlnSerLeuGlu
MetThrGluAsnPheGluThrLysAspGlyLysGInAlaGInVal
TyrProAsnArgPheProLeuTrpLysSerHisPheArgIleHis
ThrPheGluAspGluIleGluPheIleGInGlyLeuGluLysSer
ThrGlyLysLysValGlyIleTyrProGluIleLysAlaProTrp
PheHisHisGInAsnGlyLysAspIleAlaAlaGluThrLeuLys
ValLeuLysLysTyrGlyTyrAspLysLysThrAspMetValTyr
LeuGInThrPheAspPheAsnGluLeuLysArgIleLysThrGlu
LeuLeuProGInMetGlyMetAspLeuLysLeuValGInLeuIle
AlaTyrThrAspTrpLysGluThrGInGluLysAspProLysGly
TyrTrpValAsnTyrAsnTyrAspTrpMetPheLysProGlyAla
MetAlaGluValValLysTyrAlaAspGlyValGlyProGlyTrp
TyrMetLeuValAsnLysGluGluSerLysProAspAsnIleVal
TyrThrProLeuValLysGluLeuAlaGInTyrAsnValGluVal
HisProTyrThrValArgLysAspAlaLeuProGluPhePheThr
AspValAsnGlnMetTyrAspAlaLeuLeuAsnLysSerGlyAla
ThrGlyValPheThrAspPheProAspThrGlyValGluPheLeu
LysGlyIleLys
SEQ ID NO: 2:
Protein D fragment with MDP tripeptide
from NS1 (348 amino acids)
MetAspProSerSerHisSerSerAsnMetAlaAsnThrGInMet
LysSerAspLysIleIleIleAlaHisArgGlyAlaSerGlyTyr
LeuProGluHisThrLeuGluSerLysAlaLeuAlaPheAlaGIn
GlnAlaAspTyrLeuGluGInAspLeuAlaMetThrLysAspGly
ArgLeuValValIleHisAspHisPheLeuAspGlyLeuThrAsp
ValAlaLysLysPheProHisArgHisArgLysAspGlyArgTyr
TyrValIleAspPheThrLeuLysGluIleGlnSerLeuGluMet
ThrGluAsnPheGluThrLysAspGlyLysGInAlaGInValTyr
ProAsnArgPheProLeuTrpLysSerHisPheArgIleHisThr
PheGluAspGluIleGluPheIleGInGlyLeuGluLysSerThr
GlyLysLysValGlyIleTyrProGluIleLysAlaProTrpPhe
HisHisGInAsnGlyLysAspIleAlaAlaGluThrLeuLysVal
LeuLysLysTyrGlyTyrAspLysLysThrAspMetValTyrLeu
GInThrPheAspPheAsnGluLeuLysArgIleLysThrGluLeu
LeuProGInMetGlyMetAspLeuLysLeuValGInLeuIleAla
TyrThrAspTrpLysGluThrGInGluLysAspProLysGlyTyr
TrpValAsnTyrAsnTyrAspTrpMetPheLysProGlyAlaMet
AlaGluValValLysTyrAlaAspGlyValGlyProGlyTrpTyr
MetLeuValAsnLysGluGluSerLysProAspAsnIleValTyr
ThrProLeuValLysGluLeuAlaGInTyrAsnValGluValHis
ProTyrThrValArgLysAspAlaLeuProGluPhePheThrAsp
ValAsnGInMetTyrAspAlaLeuLeuAsnLysSerGlyAlaThr
GlyValPheThrAspPheProAspThrGlyValGluPheLeuLys
GlyIleLys
SEQ ID NO: 3:
SerSerHisSerSerAsnMetAlaAsnThr
SEQ ID NO: 4:
Protein E from H. influenzae
MKKIILTLSL GLLTACSAQI QKAEQNDVKL
APPTDVRSGY IRLVKNVNYY IDSESIWVDN
QEPQIVHFDA VVNLDKGLYV YPEPKRYARS
VRQYKILNCA NYHLTQVRTD FYDEFWGQGL
RAAPKKQKKH TLSLTPDTTL YNAAQIICAN
YGEAFSVDKK
SEQ ID NO: 5:
Amino acids 20-160 of Protein E
I QKAEQNDVKL APPTDVRSGY IRLVKNVNYY
IDSESIWVDN QEPQIVHFDA VVNLDKGLYV
YPEPKRYARS VRQYKILNCA NYHLTQVRTD
FYDEFWGQGL RAAPKKQKKH TLSLTPDTTL
YNAAQIICAN YGEAFSVDKK
SEQ ID NO: 6
PilA from H. influenzae
MKLTTQQTLK KGFTLIELMI VIAIIAILAT
IAIPSYQNYT KKAAVSELLQ ASAPYKADVE
LCVYSTNETT NCTGGKNGIA ADITTAKGYV
KSVTTSNGAI TVKGDGTLAN MEYILQATGN
AATGVTWTTT CKGTDASLFP ANFCGSVTQ
SEQ ID NO: 7
Amino acids 40-149 of PilA from
H. influenzae strain 86-028NP
T KKAAVSELLQ ASAPYKADVE LCVYSTNETT
NCTGGKNGIA ADITTAKGYV KSVTTSNGAI
TVKGDGTLAN MEYILQATGN AATGVTWTTT
CKGTDASLFP ANFCGSVTQ
SEQ ID NO: 8: LVL735 (protein):
(pelB sp)(ProtE aa 20-160)(GG)
(PilA aa40-149)
MKYLLPTAAA GLLLLAAQPA MAIQKAEQND
VKLAPPTDVR SGYIRLVKNV NYYIDSESIW
VDNQEPQIVH FDAVVNLDKG LYVYPEPKRY
ARSVRQYKIL NCANYHLTQV RTDFYDEFWG
QGLRAAPKKQ KKHTLSLTPD TTLYNAAQII
CANYGEAFSV DKKGGTKKAA VSELLQASAP
YKADVELCVY STNETTNCTG GKNGIAADIT
TAKGYVKSVT TSNGAITVKG DGTLANMEYI
LQATGNAATG VTWTTTCKGT DASLFPANFC
GSVTQQ
SEQ ID NO: 9: PE-PilA fusion protein
without signal peptide
IQKAEQND VKLAPPTDVR SGYIRLVKNV
NYYIDSESIW VDNQEPQIVH FDAVVNLDKG
LYVYPEPKRY ARSVRQYKIL NCANYHLTQV
RTDFYDEFWG QGLRAAPKKQ KKHTLSLTPD
TTLYNAAQII CANYGEAFSV DKKGGTKKAA
VSELLQASAP YKADVELCVY STNETTNCTG
GKNGIAADIT TAKGYVKSVT TSNGAITVKG
DGTLANMEYI LQATGNAATG VTWTTTCKGT
DASLFPANFC GSVTQ
SEQ ID NO: 10: UspA2 from ATCC 25238
MKTMKLLPLKIAVTSAMIIGLGAASTANAQAKNDITLEDLPYLIKKI
DQNELEADIGDITALEKYLALSQYGNILALEELNKALEELDEDVGWN
QNDIANLEDDVETLTKNQNALAEQGEAIKEDLQGLADFVEGQEGKIL
QNETSIKKNTQRNLVNGFEIEKNKDAIAKNNESIEDLYDFGHEVAES
IGEIHAHNEAQNETLKGLITNSIENTNNITKNKADIQALENNVVEEL
FNLSGRLIDQKADIDNNINNIYELAQQQDQHSSDIKTLKKNVEEGLL
ELSGHLIDQKTDIAQNQANIQDLATYNELQDQYAQKQTEAIDALNKA
SSENTQNIEDLAAYNELQDAYAKQQTEAIDALNKASSENTQNIEDLA
AYNELQDAYAKQQTEAIDALNKASSENTQNIAKNQADIANNINNIYE
LAQQQDQHSSDIKTLAKASAANTDRIAKNKADADASFETLTKNQNTL
IEKDKEHDKLITANKTAIDANKASADTKFAATADAITKNGNAITKNA
KSITDLGTKVDGFDSRVTALDTKVNAFDGRITALDSKVENGMAAQAA
LSGLFQPYSVGKFNATAALGGYGSKSAVAIGAGYRVNPNLAFKAGAA
INTSGNKKGSYNIGVNYEF
SEQ ID NO: 11: MC-001 (protein)-(M)
(UspA2 amino acids 30-540)(ASHHHHHH)
MQAKNDITLEDLPYLIKKIDQNELEADIGDITALEKYLALSQYGNIL
ALEELNKALEELDEDVGWNQNDIANLEDDVETLTKNQNALAEQGEAI
KEDLQGLADFVEGQEGKILQNETSIKKNTQRNLVNGFEIEKNKDAIA
KNNESIEDLYDFGHEVAESIGEIHAHNEAQNETLKGLITNSIENTNN
ITKNKADIQALENNVVEELFNLSGRLIDQKADIDNNINNIYELAQQQ
DQHSSDIKTLKKNVEEGLLELSGHLIDQKTDIAQNQANIQDLATYNE
LQDQYAQKQTEAIDALNKASSENTQNIEDLAAYNELQDAYAKQQTEA
IDALNKASSENTQNIEDLAAYNELQDAYAKQQTEAIDALNKASSENT
QNIAKNQADIANNINNIYELAQQQDQHSSDIKTLAKASAANTDRIAK
NKADADASFETLTKNQNTLIEKDKEHDKLITANKTAIDANKASADTK
FAATADAITKNGNAITKNAKSITDLGTKVDGFDSRVTALDTKASHHH
HHH
SEQ ID NO: 12
MC-002 (Protein)-(M)(UspA2 amino acids 30-540)
MQAKNDITLEDLPYLIKKIDQNELEADIGDITALEKYLALSQYGNIL
ALEELNKALEELDEDVGWNQNDIANLEDDVETLTKNQNALAEQGEAI
KEDLQGLADFVEGQEGKILQNETSIKKNTQRNLVNGFEIEKNKDAIA
KNNESIEDLYDFGHEVAESIGEIHAHNEAQNETLKGLITNSIENTNN
ITKNKADIQALENNVVEELFNLSGRLIDQKADIDNNINNIYELAQQQ
DQHSSDIKTLKKNVEEGLLELSGHLIDQKTDIAQNQANIQDLATYNE
LQDQYAQKQTEAIDALNKASSENTQNIEDLAAYNELQDAYAKQQTEA
IDALNKASSENTQNIEDLAAYNELQDAYAKQQTEAIDALNKASSENT
QNIAKNQADIANNINNIYELAQQQDQHSSDIKTLAKASAANTDRIAK
NKADADASFETLTKNQNTLIEKDKEHDKLITANKTAIDANKASADTK
FAATADAITKNGNAITKNAKSITDLGTKVDGFDSRVTALDTK
SEQ ID NO: 13
MC-003 (Protein)-(M)
(UspA2 amino acids 30-540)(H)
MQAKNDITLEDLPYLIKKIDQNELEADIGDITALEKYLALSQYGNIL
ALEELNKALEELDEDVGWNQNDIANLEDDVETLTKNQNALAEQGEAI
KEDLQGLADFVEGQEGKILQNETSIKKNTQRNLVNGFEIEKNKDAIA
KNNESIEDLYDFGHEVAESIGEIHAHNEAQNETLKGLITNSIENTNN
ITKNKADIQALENNVVEELFNLSGRLIDQKADIDNNINNIYELAQQQ
DQHSSDIKTLKKNVEEGLLELSGHLIDQKTDIAQNQANIQDLATYNE
LQDQYAQKQTEAIDALNKASSENTQNIEDLAAYNELQDAYAKQQTEA
IDALNKASSENTQNIEDLAAYNELQDAYAKQQTEAIDALNKASSENT
QNIAKNQADIANNINNIYELAQQQDQHSSDIKTLAKASAANTDRIAK
NKADADASFETLTKNQNTLIEKDKEHDKLITANKTAIDANKASADTK
FAATADAITKNGNAITKNAKSITDLGTKVDGFDSRVTALDTKH
SEQ ID NO: 14
MC-004 (Protein)-(M)(UspA2 amino acids 30-540)
(HH)
MQAKNDITLEDLPYLIKKIDQNELEADIGDITALEKYLALSQYGNIL
ALEELNKALEELDEDVGWNQNDIANLEDDVETLTKNQNALAEQGEAI
KEDLQGLADFVEGQEGKILQNETSIKKNTQRNLVNGFEIEKNKDAIA
KNNESIEDLYDFGHEVAESIGEIHAHNEAQNETLKGLITNSIENTNN
ITKNKADIQALENNVVEELFNLSGRLIDQKADIDNNINNIYELAQQQ
DQHSSDIKTLKKNVEEGLLELSGHLIDQKTDIAQNQANIQDLATYNE
LQDQYAQKQTEAIDALNKASSENTQNIEDLAAYNELQDAYAKQQTEA
IDALNKASSENTQNIEDLAAYNELQDAYAKQQTEAIDALNKASSENT
QNIAKNQADIANNINNIYELAQQQDQHSSDIKTLAKASAANTDRIAK
NKADADASFETLTKNQNTLIEKDKEHDKLITANKTAIDANKASADTK
FAATADAITKNGNAITKNAKSITDLGTKVDGFDSRVTALDTKHH
SEQ ID NO: 15
MC-005 (Protein)-(M)(UspA2 amino acids 30-519)
(ASHHHHHH)
MQAKNDITLEDLPYLIKKIDQNELEADIGDITALEKYLALSQYGNIL
ALEELNKALEELDEDVGWNQNDIANLEDDVETLTKNQNALAEQGEAI
KEDLQGLADFVEGQEGKILQNETSIKKNTQRNLVNGFEIEKNKDAIA
KNNESIEDLYDFGHEVAESIGEIHAHNEAQNETLKGLITNSIENTNN
ITKNKADIQALENNVVEELFNLSGRLIDQKADIDNNINNIYELAQQQ
DQHSSDIKTLKKNVEEGLLELSGHLIDQKTDIAQNQANIQDLATYNE
LQDQYAQKQTEAIDALNKASSENTQNIEDLAAYNELQDAYAKQQTEA
IDALNKASSENTQNIEDLAAYNELQDAYAKQQTEAIDALNKASSENT
QNIAKNQADIANNINNIYELAQQQDQHSSDIKTLAKASAANTDRIAK
NKADADASFETLTKNQNTLIEKDKEHDKLITANKTAIDANKASADTK
FAATADAITKNGNAITKNAKSASHHHHHH
SEQ ID NO: 16
MC-006 (Protein)-(M)(UspA2 amino acids 30-519)
MQAKNDITLEDLPYLIKKIDQNELEADIGDITALEKYLALSQYGNIL
ALEELNKALEELDEDVGWNQNDIANLEDDVETLTKNQNALAEQGEAI
KEDLQGLADFVEGQEGKILQNETSIKKNTQRNLVNGFEIEKNKDAIA
KNNESIEDLYDFGHEVAESIGEIHAHNEAQNETLKGLITNSIENTNN
ITKNKADIQALENNVVEELFNLSGRLIDQKADIDNNINNIYELAQQQ
DQHSSDIKTLKKNVEEGLLELSGHLIDQKTDIAQNQANIQDLATYNE
LQDQYAQKQTEAIDALNKASSENTQNIEDLAAYNELQDAYAKQQTEA
IDALNKASSENTQNIEDLAAYNELQDAYAKQQTEAIDALNKASSENT
QNIAKNQADIANNINNIYELAQQQDQHSSDIKTLAKASAANTDRIAK
NKADADASFETLTKNQNTLIEKDKEHDKLITANKTAIDANKASADTK
FAATADAITKNGNAITKNAKS
SEQ ID NO: 17
MC-007 (Protein)-(M)
(UspA2 amino acids 30-564)(ASHHHHHH)
MQAKNDITLEDLPYLIKKIDQNELEADIGDITALEKYLALSQYGNIL
ALEELNKALEELDEDVGWNQNDIANLEDDVETLTKNQNALAEQGEAI
KEDLQGLADFVEGQEGKILQNETSIKKNTQRNLVNGFEIEKNKDAIA
KNNESIEDLYDFGHEVAESIGEIHAHNEAQNETLKGLITNSIENTNN
ITKNKADIQALENNVVEELFNLSGRLIDQKADIDNNINNIYELAQQQ
DQHSSDIKTLKKNVEEGLLELSGHLIDQKTDIAQNQANIQDLATYNE
LQDQYAQKQTEAIDALNKASSENTQNIEDLAAYNELQDAYAKQQTEA
IDALNKASSENTQNIEDLAAYNELQDAYAKQQTEAIDALNKASSENT
QNIAKNQADIANNINNIYELAQQQDQHSSDIKTLAKASAANTDRIAK
NKADADASFETLTKNQNTLIEKDKEHDKLITANKTAIDANKASADTK
FAATADAITKNGNAITKNAKSITDLGTKVDGFDSRVTALDTKVNAFD
GRITALDSKVENGMAAQAAASHHHHHH
SEQ ID NO: 18
MC-008 (Protein)-(M)(UspA2 30-564)(HH)
MQAKNDITLEDLPYLIKKIDQNELEADIGDITALEKYLALSQYGNIL
ALEELNKALEELDEDVGWNQNDIANLEDDVETLTKNQNALAEQGEAI
KEDLQGLADFVEGQEGKILQNETSIKKNTQRNLVNGFEIEKNKDAIA
KNNESIEDLYDFGHEVAESIGEIHAHNEAQNETLKGLITNSIENTNN
ITKNKADIQALENNVVEELFNLSGRLIDQKADIDNNINNIYELAQQQ
DQHSSDIKTLKKNVEEGLLELSGHLIDQKTDIAQNQANIQDLATYNE
LQDQYAQKQTEAIDALNKASSENTQNIEDLAAYNELQDAYAKQQTEA
IDALNKASSENTQNIEDLAAYNELQDAYAKQQTEAIDALNKASSENT
QNIAKNQADIANNINNIYELAQQQDQHSSDIKTLAKASAANTDRIAK
NKADADASFETLTKNQNTLIEKDKEHDKLITANKTAIDANKASADTK
FAATADAITKNGNAITKNAKSITDLGTKVDGFDSRVTALDTKVNAFD
GRITALDSKVENGMAAQAAHH
SEQ ID NO: 19
MC-009 (Protein)-(M)(UspA2 31-564)(HH)
MAKNDITLEDLPYLIKKIDQNELEADIGDITALEKYLALSQYGNILA
LEELNKALEELDEDVGWNQNDIANLEDDVETLTKNQNALAEQGEAIK
EDLQGLADFVEGQEGKILQNETSIKKNTQRNLVNGFEIEKNKDAIAK
NNESIEDLYDFGHEVAESIGEIHAHNEAQNETLKGLITNSIENTNNI
TKNKADIQALENNVVEELFNLSGRLIDQKADIDNNINNIYELAQQQD
QHSSDIKTLKKNVEEGLLELSGHLIDQKTDIAQNQANIQDLATYNEL
QDQYAQKQTEAIDALNKASSENTQNIEDLAAYNELQDAYAKQQTEAI
DALNKASSENTQNIEDLAAYNELQDAYAKQQTEAIDALNKASSENTQ
NIAKNQADIANNINNIYELAQQQDQHSSDIKTLAKASAANTDRIAKN
KADADASFETLTKNQNTLIEKDKEHDKLITANKTAIDANKASADTKF
AATADAITKNGNAITKNAKSITDLGTKVDGFDSRVTALDTKVNAFDG
RITALDSKVENGMAAQAAHH
SEQ ID NO:20
MC-010 (Protein)-(M)(UspA2 amino acids 30-564)
MQAKNDITLEDLPYLIKKIDQNELEADIGDITALEKYLALSQYGNIL
ALEELNKALEELDEDVGWNQNDIANLEDDVETLTKNQNALAEQGEAI
KEDLQGLADFVEGQEGKILQNETSIKKNTQRNLVNGFEIEKNKDAIA
KNNESIEDLYDFGHEVAESIGEIHAHNEAQNETLKGLITNSIENTNN
ITKNKADIQALENNVVEELFNLSGRLIDQKADIDNNINNIYELAQQQ
DQHSSDIKTLKKNVEEGLLELSGHLIDQKTDIAQNQANIQDLATYNE
LQDQYAQKQTEAIDALNKASSENTQNIEDLAAYNELQDAYAKQQTEA
IDALNKASSENTQNIEDLAAYNELQDAYAKQQTEAIDALNKASSENT
QNIAKNQADIANNINNIYELAQQQDQHSSDIKTLAKASAANTDRIAK
NKADADASFETLTKNQNTLIEKDKEHDKLITANKTAIDANKASADTK
FAATADAITKNGNAITKNAKSITDLGTKVDGFDSRVTALDTKVNAFD
GRITALDSKVENGMAAQAA
SEQ ID NO: 21
MC-011 (Protein)-(M)(UspA2 amino acids 31-540)
(ASHHHHHH)
MAKNDITLEDLPYLIKKIDQNELEADIGDITALEKYLALSQYGNILA
LEELNKALEELDEDVGWNQNDIANLEDDVETLTKNQNALAEQGEAIK
EDLQGLADFVEGQEGKILQNETSIKKNTQRNLVNGFEIEKNKDAIAK
NNESIEDLYDFGHEVAESIGEIHAHNEAQNETLKGLITNSIENTNNI
TKNKADIQALENNVVEELFNLSGRLIDQKADIDNNINNIYELAQQQD
QHSSDIKTLKKNVEEGLLELSGHLIDQKTDIAQNQANIQDLATYNEL
QDQYAQKQTEAIDALNKASSENTQNIEDLAAYNELQDAYAKQQTEAI
DALNKASSENTQNIEDLAAYNELQDAYAKQQTEAIDALNKASSENTQ
NIAKNQADIANNINNIYELAQQQDQHSSDIKTLAKASAANTDRIAKN
KADADASFETLTKNQNTLIEKDKEHDKLITANKTAIDANKASADTKF
AATADAITKNGNAITKNAKSITDLGTKVDGFDSRVTALDTKASHHHH
HH
SEQ ID NO: 22
UspA2 American 2933 (613 aa)
MKTMKLLPLKIAVTSAMIIGLGAASTANAQSRDRSLEDIQDSISKLV
QDDINTLKQDQQKMNKYLLLNQLANTLITDELNNNVIKNTNSIEALG
DEIGWLENDIADLEEGVEELTKNQNTLIEKDEEHDRLIAQNQADIQT
LENNVVEELFNLSGRLIDQEADIAKNNASIEELYDFDNEVAERIGEI
HAYTEEVNKTLENLITNSVKNTDNIDKNKADIDNNINHIYELAQQQD
QHSSDIKTLKNNVEEGLLELSGHLIDQKADLTKDIKALESNVEEGLL
DLSGRLLDQKADLTKDIKALESNVEEGLLDLSGRLLDQKADIAQNQT
DIQDLAAYNELQDQYAQKQTEAIDALNKASSENTQNIEDLAAYNELQ
DAYAKQQTEAIDALNKASSENTQNIAKNQADIANNINNIYELAQQQD
QHSSDIKTLAKASAANTNRIATAELGIAENKKDAQIAKAQANANKTA
IDENKASADTKFAATADAITKNGNAITKNAKSITDLGTKVDGFDGRV
TALDTKVNAFDGRITALDSKVENGMAAQAALSGLFQPYSVGKFNATA
ALGGYGSKSAVAIGAGYRVNPNLAFKAGAAINTSGNKKGSYNIGVNY
EF
SEQ ID NO: 23
UspA2 American 2912 (644 aa)
MKTMKLLPLKIAVTSALIIGLGAASTANAQQQLQTETFLPNFLSNDN
YDLTDPFYHNMILGDTALLDKQDGSQPQLKFYSNDKDSVPDSLLFSK
LLHEQQLNGFKKGDTIIPLDKDGKPVYQVDYKLDGKGKKQKRRQVYS
VTTKTATDDDVNSAYSRGILGKVDDLDDEMNFLNHDITSLYDVTANQ
QDAIKDLKKGVKGLNKELKELDKEVGVLSRDIGSLNDDVAQNNESIE
DLYDFSQEVADSIGEIHAHNKAQNETLQDLITNSVENTNNITKNKAD
IQALENNVVEELFNLSGRLIDQKADLTKDIKTLESNVEEGLLELSGH
LIDQKADIAKNQADIAQNQANIQDLAAYNELQDAYAKQQTEAIDALN
KASSENTQNIEDLAAYNELQDAYAKQQTEAIDALNKASSENTQNIAK
NQADIANNINNIYELAQQQDQHSSDIKTLAKASAANTDRIAKNKADA
DASFETLTKNQNTLIEKDKEHDKLITANKTAIDENKASADTKFAATA
DAITKNGNAITKNAKSITDLGTKVDGFDSRVTALDTKVNAFDGRITA
LDSKVENGMAAQAALSGLFQPYSVGKFNATAALGGYGSKSAVAIGAG
YRVNPNLAFKAGAAINTSGNKKGSYNIGVNYEF
SEQ ID NO: 24
UspA2 American 2908 (591 aa)
MKTMKLLPLKIAVTSALIVGLGAASTANAQLVERFFPNIFLDKPLAK
QHYHNVVVGDTSIVSDLQSNSDQLKFYSDDEGLVPDSLLFNKMLHEQ
LLNGFKEGDTIIPLDENGKPVYKVDYKLDGKEPRKVYSVTTKIATAE
DVATSSYANGIQKDIDDLYDFDHQVTERLTQHGKTIYRNGERILANE
ESVQYLNKEVQNNIEHIYELAQQQDQHSSDIKTLESNVEKGLLELSG
HLIDQKADLTKDIKTLESNVEEGLLDLSGRLIDQKADLTKDIKTLES
NVEEGLLDLSGRLIDQKADIAQNQANIQDLAAYNELQDQYAQKQTEA
IDALNKASSENTQNIEDLAAYNELQDAYAKQQTEAIDALNKASSENT
QNIAKNQADIANNINNIYELAQQQDQHSSDIKTLAKASAANTNRIAT
AELGIAENKKDAQIAKAQANANKTAIDENKASADTKFAATADAITKN
GNAITKNAKSITDLGTKVDGFDSRVTALDTKVNAFDGRITALDSKVE
NGMAAQAALSGLFQPYSVGKFNATAALGGYGSKSAVAIGAGYRVNPN
LAFKAGAAINTSGNKKGSYNIGVNYEF
SEQ ID NO: 25
UspA2 Finnish 307 (687 aa)
MKTMKLLPLKIAVTSAMIIGLGAASTANAQQQQQQQQQQQSRTEIFF
PNIFFNENHDELDDAYHNIILGDTALLDKQDGSQPQLKFYSNDKDSV
PDSLLFSKLLHEQQLNGFKKGDTIIPLDKDGKPVYQVDYKLDGKGKK
QKRRQVYSVTTKTATDDDVNSAYSRGILGKVDDLDDEMNFLNHDITS
LYDVTANQQDAIKGLKKGVKGLNKELKELDKEVGVLSRDIGSLNDDV
AQNNESIEDLYDFSQEVADSIGEIHAHNKAQNETLQDLITNSVENTN
NITKNKADIQALENNVVEELFNLSGRLIDQKADLTKDIKTLESNVEE
GLLELSGHLIDQKADIAKNQADIAQNQANIQDLAAYNELQDAYAKQQ
TEAIDALNKASSENTQNIEDLAAYNELQDAYAKQQTEAIDALNKASS
ENTQNIEDLAAYNELQDAYAKQQTEAIDALNKASSENTQNIAKNQAD
IANNINNIYELAQQQDQHSSDIKTLAKASAANTDRIAKNKADADASF
ETLTKNQNTLIEKDKEHDKLITANKTAIDENKASADTKFAATADAIT
KNGNAITKNAKSITDLGTKVDAFDGRVTALDTKVNAFDGRITALDSK
VENGMAAQAALSGLFQPYSVGKFNATAALGGYGSKSAVAIGAGYRVN
PNLAFKAGAAINTSGNKKGSYNIGVNYEF
SEQ ID NO: 26
UspA2 Finnish 353 (683 amino acids)
MKTMKLLPLKIAVTSAMIVGLGMASTANAQQQKSPKTETFLPNIFFN
EYADDLDTLYHNMILGDTAITHDDQYKFYADDATEVPDSLFFNKILH
DQLLYGFKEGDKIIPLDENGKPVYKLDKRLENGVQKTVYSVTTKTAT
ADDVNSAYSRGIQGDIDDLYEANKENVNRLIEHGDKIFANEESVQYL
NREVQNNIENIHELAQQQDQHSSDIKTLKKNVEKDLLDLSGRLIAQK
EDIAQNQTDIQDLATYNELQDQYAQKQTEAIDALNKASSENTQNIAK
NSNHIKTLENNIEEGLLELSGHLIDQKADLTKDIKALESNVEEGLLD
LSGRLIDQKADIAQNQANIQDLAAYNELQDAYAKQQTEAIDALNKAS
SENTQNIEDLAAYNELQDAYAKQQTEAIDALNKASSENTQNIEDLAA
YNELQDAYAKQQTEAIDALNKASSENTQNIAKNQADIANNINNIYEL
AQQQDQHSSDIKTLAKASAANTDRIAKNKADADASFETLTKNQNTLI
EKDKEHDKLITANKTAIDANKASADTKFAATADAITKNGNAITKNAK
SITDLGTKVDGFDGRVTALDTKVNALDTKVNAFDGRITALDSKVENG
MAAQAALSGLFQPYSVGKFNATAALGGYGSKSAVAIGAGYRVNPNLA
FKAGAAINTSGNKKGSYNIGVNYEF
SEQ ID NO: 27
UspA2 Finnish 358 (684 amino acids)
MKTMKLLPLKIAVTSAMMVGLGMASTANAQQQKSPKTEIFLPNLFDN
DNTELTDPLYHNMILGNTALLTQENQYKFYADDGNGVPDSLLFNKIL
HDQLLHGFKEGGTIIPLDENGKPVYKLDSIVEQGKTKTVYSVTTKTA
TADDVNSAYSRGIQGDIDDLYEANKENVNRLIEHGDKIFANEESVQY
LNREVQNNIENIHELAQQQDQHSSDIKTLKKNVEKDLLDLSGRLIAQ
KEDIAQNQTDIQDLATYNELQDQYAQKQTEAIDALNKASSENTQNIA
KNSNHIKTLENNIEEGLLELSGHLIDQKADLTKDIKALESNVEEGLL
DLSGRLIDQKADIAQNQANIQDLAAYNELQDAYAKQQTEAIDALNKA
SSENTQNIEDLAAYNELQDAYAKQQTEAIDALNKASSENTQNIEDLA
AYNELQDAYAKQQTEAIDALNKASSENTQNIAKNQADIANNINNIYE
LAQQQDQHSSDIKTLAKASAANTDRIAKNKADADASFETLTKNQNTL
IEKDKEHDKLITANKTAIDANKASADTKFAATADAITKNGNAITKNA
KSITDLGTKVDGFDGRVTALDTKVNALDTKVNAFDGRITALDSKVEN
GMAAQAALSGLFQPYSVGKFNATAALGGYGSKSAVAIGAGYRVNPNL
AFKAGAAINTSGNKKGSYNIGVNYEF
SEQ ID NO: 28
UspA2 Finnish 216 (684 amino acids)
MKTMKLLPLKIAVTSAMIIGLGAASTANAQQQQKTKTEVFLPNLFDN
DYYDLTDPLYHSMILGDTATLFDQQDNSKSQLKFYSNDKDSVPDSLL
FSKLLHEQQLNGFKAGDTIIPLDKDGKPVYTQDTRTKDGKVETVYSV
TTKIATQDDVEQSAYSRGIQGDIDDLYDINREVNEYLKATHDYNERQ
TEAIDALNKASSANTDRIDTAEERIDKNEYDIKALESNVGKDLLDLS
GRLIAQKEDIDNNINHIYELAQQQDQHSSDIKTLKNNVEEGLLELSG
HLIDQKADLTKDIKTLENNIEEGLLELSGHLIDQKADLTKDIKTLEN
NIEEGLLELSGHLIDQKADIAQNQANIQDLAAYNELQDQYAQKQTEA
IDALNKASSENTQNIEDLAAYNELQDAYAKQQTEAIDALNKASSENT
QNIEDLAAYNELQDAYAKQQTEAIDALNKASSENTQNIAKNQADIAN
NINNIYELAQQQDQHSSDIKTLAKVSAANTDRIAKNKADADASFETL
TKNQNTLIEKDKEHDKLITANKTAIDANKASADTKFAATADAITKNG
NAITKNAKSITDLGTKVDGFDGRVTALDTKVNAFDGRITALDSKVEN
GMAAQAALSGLFQPYSVGKFNATAALGGYGSKSAVAIGAGYRVNPNL
AFKAGAAINTSGNKKGSYNIGVNYEF
SEQ ID NO: 29
UspA2 Dutch H2 (684 amino acids)
MKTMKLLPLKIAVTSAMMVGLGMASTANAQQQKSPKTEIFLPNLFDN
DNTELTDPLYHNMILGNTALLTQENQYKFYADDGNGVPDSLLFNKIL
HDQLLHGFKKGDTIIPLDENGKPVYKLDSIVEQGKTKTVYSVTTKTA
TADDVNSAYSRGIQGDIDDLYEANKENVNRLIEHGDKIFANEESVQY
LNREVQNNIENIYELVQQQDQHSSDIKTLKKNVEKDLLDLSGRLIAQ
KEDIAQNQTDIQDLATYNELQDQYAQKQTEAIDALNKASSENTQNIA
KNSNHIKTLENNIEEGLLELSGHLIDQKADLTKDIKALESNVEEGLL
DLSGRLIDQKADIAQNQANIQDLAAYNELQDAYAKQQTEAIDALNKA
SSENTQNIEDLAAYNELQDAYAKQQTEAIDALNKASSENTQNIEDLA
AYNELQDAYAKQQTEAIDALNKASSENTQNIAKNQADIANNINNIYE
LAQQQDQHSSDIKTLAKASAANTDRIAKNKADADASFETLTKNQNTL
IEKDKEHDKLITANKTAIDANKASADTKFAATADAITKNGNAITKNA
KSITDLGTKVDGFDGRVTALDTKVNALDTKVNAFDGRITALDSKVEN
GMAAQAALSGLFQPYSVGKFNATAALGGYGSKSAVAIGAGYRVNPNL
AFKAGAAINTSGNKKGSYNIGVNYEF
SEQ ID NO: 30
UspA2 Dutch F10 (574 amino acids)
MKTMKLLPLKIAVTSAMIIGLGAASTANAQLAEQFFPNIFSNHAPVK
QHYHNVVVGDTSIVENLQDSDDTQLKFYSNDEYSVPDSLLFNKMLHE
QQLNGFKKGDTIIPLDENGKPVYKVDYKLDGQEPRRVYSVTTKIATQ
DDVDNSPYSRGIQGDIDDLYEANKENVNRLIEHGDKIFANEESVQYL
NKEVQNNIENIYELAQQQDQHSSDIKTLKKNVEEGLLELSGHLIDQK
ADLTKDIKTLESNVEEGLLELSGHLIDQKADIAKNQADIAQNQANIQ
DLAAYNELQDAYAKQQTEAIDALNKASSENTQNIEDLAAYNELQDAY
AKQQTEAIDALNKASSENTQNIAKNQADIANNINNIYELAQQQDQHS
SDIKTLAKASAANTDRIAKNKADADASFETLTKNQNTLIEKDKEHDK
LITANKTAIDANKASADTKFAATADAITKNGNAITKNAKSITDLGTK
VDAFDGRVTALDTKVNAFDGRITALDSKVENGMAAQAALSGLFQPYS
VGKFNATAALGGYGSKSAVAIGAGYRVNPNLAFKAGAAINTSGNKKG
SYNIGVNYEF
SEQ ID NO: 31
UspA2 Norwegian 1(678 amino acids)
MKTMKLLPLKIAVTSALIVGLGAASTANAQQQPQTETFFPNIFFNEN
HDALDDVYHNMILGDTAITQDNQYKFYADAISEVPDSLLFNKILHDQ
QLNGFKEGDTIIPLDENGKPVYKLDEKVENGVKKSVYSVTTKTATRA
DVEQSAYSRGIQGDIDDLYEANKENVNRLIEHGDKIFANEESVQYLN
KEVQNNIENIHELAQQQDQHSSDIKTLKKNVEEGLLELSGHLIDQKA
DLTKDIKTLESNVEEGLLDLSGRLLDQKADIAQNQANIQDLAAYNEL
QDAYAKQQTEAIDALNKASSENTQNIEDLAAYNELQDAYAKQQTEAI
DALNKASSENTQNIEDLAAYNELQDAYAKQQTEAIDALNKASSENTQ
NIEDLAAYNELQDAYAKQQTEAIDALNKASSENTQNIEDLAAYNELQ
DAYAKQQTEAIDALNKASSENTQNIAKNQADIANNINNIYELAQQQD
QHSSDIKTLAKASAANTDRIAKNKADADASFETLTKNQNTLIEKDKE
HDKLITANKTAIDANKASADTKFAATADAITKNGNAITKNAKSITDL
GTKVDAFDGRVTALDTKVNALDTKVNAFDGRITALDSKVENGMAAQA
ALSGLFQPYSVGKFNATAALGGYGSKSAVAIGAGYRVNPNLAFKAGA
AINTSGNKKGSYNIGVNYEF
SEQ ID NO: 32
UspA2 Norwegian 13 (678 amino acids)
MKTMKLLPLKIAVTSAMIVGLGAASTANAQQQQQPRTETFFPNIFFN
ENHDALDDVYHNMILGDTAITQDNQYKFYADAISEVPDSLLFNKILH
DQQLNGFKEGDTIIPLDENGKPVYKLDEKVENGVKKSVYSVTTKTAT
RADVEQSAYSRGIQGDIDDLYEANKENVNRLIEHGDKIFANEESVQY
LNREVQNNIENIHELAQQQDQHSSDIKTLKKNVEKDLLDLSGRLIAQ
KEDIAQNQTDIQDLATYNELQDQYAQKQTEAIDALNKASSENTQNIA
KNSNHIKTLENNIEEGLLELSGHLIDQKADLTKDIKTLENNIEEGLL
ELSGHLIDQKADLTKDIKALESNVEEGLLDLSGRLLDQKADIAQNQA
NIQDLAAYNELQDQYAQKQTEAIDALNKASSENTQNIEDLAAYNELQ
DAYAKQQTEAIDALNKASSENTQNIAKNQADIANNINNIYELAQQQD
QHSSDIKTLAKASAANTDRIAKNKADADASFETLTKNQNTLIEKDKE
HDKLITANKTAIDTNKASADTKFAATADAITKNGNAITKNAKSITDL
GTKVDGFDGRVTALDTKVNALDTKVNAFDGRITALDSKVENGMAAQA
ALSGLFQPYSVGKFNATAALGGYGSKSAVAIGAGYRVNPNLAFKAGA
AINTSGNKKGSYNIGVNYEF
SEQ ID NO: 33
UspA2 Norwegian 33 (587 amino acids)
MKTMKLLPLKIAVTSALIVGLGAASTANAQLVERFFPNIFLDKPLAK
QHYHNVVVGDTSIVSDLQSNSDQLKFYSDDEGLVPDSLLFNKMLHEQ
LLNGFKEGDTIIPLDENGKPVYKVDYKLDGKEPRKVYSVTTKIATAE
DVATSSYANGIQKDIDDLYDFDHQVTERLTQHGKTIYRNGERILANE
ESVQYLNKEVQNNIEHIYELAQQQDQHSSDIKTLESNVEKGLLELSG
HLIDQKADLTKDIKTLENNVEEGLLDLSGRLIDQKADIAQNQANIQD
LAAYNELQDQYAQKQTEAIDALNKASSENTQNIEDLAAYNELQDAYA
KQQTEAIDALNKASSENTQNIAKNQADIANNINNIYELAQQQDQHSS
DIKTLAKASAANTDRIAKNKADADASFETLTKNQNTLIEKDKEHDKL
ITANKTAIDTNKASADTKFAATADAITKNGNAITKNAKSITDLGTKV
DGFDSRVTALDTKVNALDTKVNALDTKVNAFDGRITALDSKVENGMA
AQAALSGLFQPYSVGKFNATAALGGYGSKSAVAIGAGYRVNPNLAFK
AGAAINTSGNKKGSYNIGVNYEF
SEQ ID NO: 34
UspA2 Norwegian 25 (678 amino acids)
MKTMKLLPLKIAVTSAMIVGLGAASTANAQQQQQPRTETFFPNIFFN
ENHDALDDVYHNMILGDTAITQDNQYKFYADAISEVPDSLLFNKILH
DQQLNGFKEGDTIIPLDENGKPVYKLDEKVENGVKKSVYSVTTKTAT
RADVEQSAYSRGIQGDIDDLYEANKENVNRLIEHGDKIFANEESVQY
LNREVQNNIENIHELAQQQDQHSSDIKTLKKNVEKDLLDLSGRLIAQ
KEDIAQNQTDIQDLATYNELQDQYAQKQTEAIDALNKASSENTQNIA
KNSNHIKTLENNIEEGLLELSGHLIDQKADLTKDIKTLENNIEEGLL
ELSGHLIDQKADLTKDIKALESNVEEGLLDLSGRLLDQKADIAQNQA
NIQDLAAYNELQDQYAQKQTEAIDALNKASSENTQNIEDLAAYNELQ
DAYAKQQTEAIDALNKASSENTQNIAKNQADIANNINNIYELAQQQD
QHSSDIKTLAKASAANTDRIAKNKADADASFETLTKNQNTLIEKDKE
HDKLITANKTAIDTNKASADTKFAATADAITKNGNAITKNAKSITDL
GTKVDGFDGRVTALDTKVNALDTKVNAFDGRITALDSKVENGMAAQA
ALSGLFQPYSVGKFNATAALGGYGSKSAVAIGAGYRVNPNLAFKAGA
AINTSGNKKGSYNIGVNYEF
SEQ ID NO: 35
UspA2 Norwegian 27 (616 amino acids)
MKTMKLLPLKIAVTSALIVGLGAASTANAQVRDKSLEDIEALLGKID
ISKLEKEKKQQTELQKYLLLSQYANVLTMEELNKNVEKNTNSIEALG
YEIGWLENDIADLEEGVEELTKNQNTLIEKDEEHDRLIAQNQADIKT
LENNVVEELFNLSDRLIDQEADIAKNNASIEELYDFDNEVAERIGEI
HAYTEEVNKTLEKLITNSVKNTDNIDKNKADIQALENNVEEGLLELS
GHLIDQKADLTKDIKALESNVEEGLLDLSGRLLDQKADIAKNQADIA
QNQTDIQDLAAYNELQDQYAQKQTEAIDALNKASSENTQNIEDLAAY
NELQDAYAKQQTEAIDALNKASSENTQNIEDLAAYNELQDAYAKQQT
EAIDALNKASSENTQNIAKNQADIANNINNIYELAQQQDQHSSDIKT
LAKVSAANTDRIAKNKADADASFETLTKNQNTLIEKDKEHDKLITAN
KTAIDANKASADTKFAATADAITKNGNAITKNAKSITDLGTKVDGFD
SRVTALDTKVNAFDGRITALDSKVENGMAAQAALSGLFQPYSVGKFN
ATAALGGYGSKSAVAIGAGYRVNPNLAFKAGAAINTSGNKKGSYNIG
VNYEF
SEQ ID NO: 36
UspA2 Norwegian 36 (676 amino acids)
MKTMKLLPLKIAVTSALIVGLGAASTANAQATETFLPNLFDNDYTET
TDPLYHGMILGNTAITQDTQYKFYAENGNEVPDSLFFNKILHDQQLN
GFKEGDTIIPLDENGKPVYKLDEITENGVKRKVYSVTTKTATREDVE
QSAYSRGIQGDIDDLYEANKENVNRLIEHGDKIFANEESVQYLNKEV
QNNIENIHELAQQQDQHSSDIKTLKKNVEEGLLELSGHLIDQKADLT
KDIKALESNVEEGLLDLSGHLIDQKADLTKDIKALESNVEEGLLDLS
GRLLDQKADIAKNQADIAQNQTDIQDLAAYNELQDQYAQKQTEAIDA
LNKASSENTQNIEDLAAYNELQDQYAQKQTEAIDALNKASSENTQNI
EDLAAYNELQDQYAQKQTEAIDALNKASSENTQNIEDLAAYNELQDQ
YAQKQTEAIDALNKASSENTQNIAKNQADIANNINNIYELAQQQDQH
SSDIKTLAKASAANTDRIAKNKADADASFETLTKNQNTLIEKDKEHD
KLITANKTAIDANKASADTKFAATADAITKNGNAITKNAKSITDLGT
KVDGFDGRVTALDTKVNALDTKVNAFDGRITALDSKVENGMAAQAAL
SGLFQPYSVGKFNATAALGGYGSKSAVAIGAGYRVNPNLAFKAGAAI
NTSGNKKGSYNIGVNYEF
SEQ ID NO: 37
UspA2 BC5SV (629 amino acids)
MKTMKLLPLKIAVTSALIVGLGAASTANAQNGTSTKLKNLKEYAQYL
DNYAQYLDDDIDDLDKEVGELSQNIAKNQANIKDLNKKLSRDIDSLR
EDVYDNQYEIVNNQADIEKNQDDIKELENNVGKELLNLSGRLLDQKA
DIDNNINNIYELAQQQDQHSSDIKTLKKNVEEGLLELSGHLIDQKSD
IAQNQTDIQDLATYNELQDQYAQKQTEAIDALNKASSENTQNIEDLA
AYNELQDAYAKQQTEAIDALNKASSENTQNIQDLAAYNELQDAYAKQ
QTEAIDALNKASSENTQNIEDLAAYNELQDAYAKQQTEAIDALNKAS
SENTQNIEDLAAYNELQDAYAKQQTEAIDALNKASSENTQNIEDLAA
YNELQDAYAKQQTEAIDALNKASSENTQNIAKNQADIANNINNIYEL
AQQQDQHSSDIKTLAKASAANTDRIAKNKADADASFETLTKNQNTLI
EKDKEHDKLITANKTAIDANKASADTKFAATADAITKNGNAITKNAK
SITDLGTKVDAFDGRVTALDTKVNAFDGRITALDSKVENGMAAQAAL
SGLFQPYSVGKFNATAALGGYGSKSAVAIGAGYRVNPNLAFKAGAAI
NTSGNKKGSYNIGVNYEF
SEQ ID NO: 38
UspA2 Norwegian 14 (683 amino acids)
MKTMKLLPLKIAVTSAMIVGLGMASTANAQQQRSPKTETFLPNIFFN
EYADDLDTLYHNMILGDTAITHDDQYKFYADDATEVPDSLFFNKILH
DQLLYGFKEGDKIIPLDENGKPVYKLDKRLDNGVQKTVYSVTTKTAT
ADDVNSAYSRGIQGDIDDLYEANKENVNRLIEHGDKIFANEESVQYL
NKEVQNNIENIHELAQQQDQHSSDIKTLKKNVEEGLLELSGHLIDQK
TDIAQNQTDIQDLATYNELQDQYAQKQTEAIDALNKASSENTQNIAK
NSNRIKALENNIEEGLLELSGHLIDQKADLTKDIKALESNVEEGLLD
LSGRLIDQKADIAQNQANIQDLAAYNELQDAYAKQQTEAIDALNKAS
SENTQNIEDLAAYNELQDAYAKQQTEAIDALNKASSENTQNIEDLAA
YNELQDAYAKQQTEAIDALNKASSENTQNIAKNQADIANNINNIYEL
AQQQDQHSSDIKTLAKASAANTDRIAKNKADADASFETLTKNQNTLI
EKDKEHDKLITANKTAIDANKASADTKFAATADAITKNGNAITKNAK
SITDLGTKVDGFDGRVTALDTKVNALDTKVNAFDGRITALDSKVENG
MAAQAALSGLFQPYSVGKFNATAALGGYGSKSAVAIGAGYRVNPNLA
FKAGAAINTSGNKKGSYNIGVNYEF
SEQ ID NO: 39
UspA2 Norwegian 3 (700 amino acids)
MKTMKLLPLKIAVTSAMIVGLGAASTANAQAQSNRSLDQVQALLRGI
DETKIKKEIQQSQQPELNKYLTFNQLANALNIEELNNNVQKNTQRLD
SAATLYGDLSKTVPKSIKENKESIKENKESIKENKESIKENKESIKE
NKESIKENKESITTLTRKSFQNQVDIVRNNASIEDLYAYGQEVAKSI
GEIHAYTEEVNKTLENLITNSVENTNNITKNKADIQALENNVVEELF
NLSGRLIDQKADIDNNINNIYELAQQQDQHSSDIKTLKKNVEEGLLE
LSGHLIDQKADLTKDIKTLESNVEEGLLDLSGRLLDQKADIAQNQAN
IQDLAAYNELQDAYAKQQTEAIDALNKASSENTQNIEDLAAYNELQD
AYAKQQTEAIDALNKASSENTQNIEDLAAYNELQDAYAKQQTEAIDA
LNKASSENTQNIEDLAAYNELQDAYAKQQTEAIDALNKASSENTQNI
AKNQADIANNINNIYELAQQQDQHSSDIKTLAKASAANTDRIAKNKA
DADASFETLTKNQNTLIEKDKEHDKLITANKTVIDANKASADTKFAA
TADAITKNGNAITKNAKSITDLGTKVDGFDGRVTALDTKVNALDTKV
NAFDGRITALDSKVENGMAAQAALSGLFQPYSVGKFNATAALGGYGS
KSAVAIGAGYRVNPNLAFKAGAAINTSGNKKGSYNIGVNYEF
SEQ ID NO: 40
UspA2 Finnish 414 (676 amino acids)
MKTMKLLPLKIAVTSALIVGLGAASTANAQATETFLPNLFDNDYIET
TDPLYHGMILGNTAITQDTQYKFYAENGNEVPDSLFFNKILHDQQLN
GFKEGDTIIPLDENGKPVYKLDEITENGVKRKVYSVTTKTATREDVE
QSAYSRGIQGDIDDLYEANKENVNRLIEHGDKIFANEESVQYLNKEV
QNNIENIHELAQQQDQHSSDIKTLKKNVEEGLLELSGHLIDQKADLT
KDIKTLENNVEEGLLELSGHLIDQKADLTKDIKALESNVEEGLLDLS
GRLLDQKADIAKNQADIAQNQTDIQDLAAYNELQDQYAQKQTEAIDA
LNKASSENTQNIEDLAAYNELQDQYAQKQTEAIDALNKASSENTQNI
EDLAAYNELQDQYAQKQTEAIDALNKASSENTQNIEDLAAYNELQDQ
YAQKQTEAIDALNKASSENTQNIAKNQADIANNINNIYELAQQQDQH
SSDIKTLAKASAANTDRIAKNKADADASFETLTKNQNTLIEKDKEHD
KLITANKTAIDANKASADTKFAATADAITKNGNAITKNAKSITDLGT
KVDGFDGRVTALDTKVNALDTKVNAFDGRITALDSKVENGMAAQAAL
SGLFQPYSVGKFNATAALGGYGSKSAVAIGAGYRVNPNLAFKAGAAI
NTSGNKKGSYNIGVNYEF
SEQ ID NO: 41
UspA2 Japanese Z7476 (678 amino acids)
MKTMKLLPLKIAVTSAMIIGLGAASTANAQLAEQFFPNIFSNHAPVK
QHYHNVVVGDTSIVENLQDSDDTQLKFYSNDEYSVPDSLLFNKMLHE
QQLNGFKKGDTIIPLDENGKPVYKVDYKLDGQEPRRVYSVTTKIATQ
DDVDNSPYSRGIQGDIDDLYEANKENVNRLIEHGDKIFANEESVQYL
NKEVQNNIENIYELAQQQDQHSSDIKTLKKNVEEGLLELSGRLIDQK
ADIAQNQANIQDLAAYNELQDQYAQKQTEAIDALNKASSENTQNIED
LAAYNELQDAYAKQQTEAIDALNKASSENTQNIEDLAAYNELQDAYA
KQQTEAIDALNKASSENTQNIEDLAAYNELQDAYAKQQTEAIDALNK
ASSENTQNIEDLAAYNELQDAYAKQQTEAIDALNKASSENTQNIEDL
AAYNELQDAYAKQQTEAIDALNKASSENTQNIAKNQADIANNINNIY
ELAQQQDQHSSDIKTLAKVSAANTDRIAKNKADADASFETLTKNQNT
LIEKDKEHDKLITANKTAIDANKASADTKFAATADAITKNGNAITKN
AKSITDLGTKVDGFDGRVTALDTKVNAFDGRITALDSKVENGMAAQA
ALSGLFQPYSVGKFNATAALGGYGSKSAVAIGAGYRVNPNLAFKAGA
AINTSGNKKGSYNIGVNYEF
SEQ ID NO: 42
UspA2 Belgian Z7530 (613 amino acids)
MKTMKLLPLKIAVTSAMIIGLGAASTANAQSRDRSLEDIQDSISKLV
QDDINTLKQDQQKMNKYLLLNQLANTLITDELNNNVIKNTNSIEALG
DEIGWLENDIADLEEGVEELTKNQNTLIEKDEEHDRLIAQNQADIQT
LENNWVEELFNLSGRLIDQEADIAKNNASIEELYDFDNEVAERIGEI
HAYTEEVNKTLENLITNSVKNTDNIDKNKADIDNNINHIYELAQQQD
QHSSDIKTLKNNVEEGLLELSGHLIDQKADLTKDIKALESNVEEGLL
DLSGRLLDQKADLTKDIKALESNVEEGLLDLSGRLLDQKADIAQNQT
DIQDLAAYNELQDQYAQKQTEAIDALNKASSENTQNIEDLAAYNELQ
DAYAKQQTEAIDALNKASSENTQNIAKNQADIANNINNIYELAQQQD
QHSSDIKTLAKASAANTNRIATAELGIAENKKDAQIAKAQANANKTA
IDENKASADTKFAATADAITKNGNAITKNAKSITDLGTKVDGFDGRV
TALDTKVNAFDGRITALDSKVENGMAAQAALSGLFQPYSVGKFNATA
ALGGYGSKSAVAIGAGYRVNPNLAFKAGAAINTSGNKKGSYNIGVNY
EF
SEQ ID NO: 43
German Z8063 (589 amino acids)
MKTMKLLPLKIAVTSALIVGLGAASTANAQATNKDITLEDVLKSIEE
IDPYELRDYIEYPTAIERFLLLSQYGNTLTLEEFDNDIELLDQDVED
LEESVTELAKNQNSLIEQGEAIKEDLQGLADFVERQEDKILQNETSI
KKNTQRNLVNGFEIEKNKDAIAKNNESIEDLYDFGHEVAKSIGEIHA
HNEAQNETLKDLITNSVKNTDNITKNKADIQALESNVEKGLLELSGH
LIDQKADIDNNINNIHELAQQQDQHSSDIKTLKKNVEEGLLELSGHL
IDQKSDIAQNQANIQDLATYNELQDQYAQKQTEAIDALNKASSENTQ
NIEDLAAYNELQDAYAKQQTEAIDALNKASSENTQNIAKNQADIANN
INNIYELAQQQDQHSSDIKTLAKASAANTDRIAKNKADADASFETLT
KNQNTLIEKDKEHDKLITANKTAIDANKASADTKFAATADAITKNGN
AITKNAKSITDLGTKVDGFDSRVTALDTKVNAFDGRITALDSKVENG
MAAQAALSGLFQPYSVGKFNATAALGGYGSKSAVAIGAGYRVNPNLA
FKAGAAINTSGNKKGSYNIGVNYEF
SEQ ID NO: 44
UspA2 American O12E (684 amino acids)
MKTMKLLPLKIAVTSAMMVGLGMASTANAQQQKSPKTEIFLPNLFDN
DNTELTDPLYHNMILGNTALLTQENQYKFYADDGNGVPDSLLFNKIL
HDQLLHGFKEGDTIIPLDENGKPVYKLDSIVEQGKTKTVYSVTTKTA
TADDVNSAYSRGIQGDIDDLYEANKENVNRLIEHGDKIFANEESVQY
LNREVQNNIENIHELAQQQDQHSSDIKTLKKNVEKDLLDLSGRLIAQ
KEDIAQNQTDIQDLATYNELQDQYAQKQTEAIDALNKASSENTQNIA
KNSNHIKTLENNIEEGLLELSGHLIDQKADLTKDIKALESNVEEGLL
DLSGRLIDQKADIAQNQANIQDLAAYNELQDAYAKQQTEAIDALNKA
SSENTQNIEDLAAYNELQDAYAKQQTEAIDALNKASSENTQNIEDLA
AYNELQDAYAKQQTEAIDALNKASSENTQNIAKNQADIANNINNIYE
LAQQQDQHSSDIKTLAKASAANTDRIAKNKADADASFETLTKNQNTL
IEKDKEHDKLITANKTAIDANKASADTKFAATADAITKNGNAITKNA
KSITDLGTKVDGFDGRVTALDTKVNALDTKVNAFDGRITALDSKVEN
GMAAQAALSGLFQPYSVGKFNATAALGGYGSKSAVAIGAGYRVNPNL
AFKAGAAINTSGNKKGSYNIGVNYEF
SEQ ID NO: 45
UspA2 Greek MC317 (650 amino acids)
MKTMKLLPLKIAVTSALIVGLGAASTANAQQQQKTKTEVFLPNLFYN
DYIEETDLLYHNMILGDTAALVDRQNYSNSQLKFYSNDEESVPDSLL
FSKMLNNQQLNGFKAGDIIIPVDANGQVIYQKDTRVEGGKTRTVLSV
TTKIATQQDVDSAYSRGIQGKVNDLDDEMNFLNHDITSLYDVTANQQ
DDIKGLKKGVKDLKKGVKGLNKELKELDKEVGVLSRDIGSLNDDVAQ
NNESIEDLYDFSQEVADSIGEIHAHNKAQNETLQDLITNSVENTNNI
TKNKADIQALENNVVEELFNLSGRLIDQKADLTKDIKTLESNVEEGL
LELSGHLIDQKADIAKNQADIAQNQANIQDLAAYNELQDAYAKQQTE
AIDALNKASSENTQNIEDLAAYNELQDAYAKQQTEAIDALNKASSEN
TQNIAKNQADIANNINNIYELAQQQDQHSSDIKTLAKASAANTDRIA
KNKADADASFETLTKNQNTLIEKDKEHDKLITANKTAIDENKASADT
KFAATADAITKNGNAITKNAKSITDLGTKVDGFDGRVTALDTKVNAF
DGRITALDSKVENGMAAQAALSGLFQPYSVGKFNATAALGGYGSKSA
VAIGAGYRVNPNLAFKAGAAINTSGNKKGSYNIGVNYEF
SEQ ID NO: 46
UspA2 American V1122 (616 amino acids)
MKTMKLLPLKIAVTSALIVGLGAVSTTNAQAQSRSLDQIQTKLADLA
GKIAAGKNGGGQNNQNNQNDINKYLFLSQYANILTMEELNNNVVKNS
SSIETLETDFGWLENDVADLEDGVEELTKNQNTLIEKDEEHDRLIAQ
NQADIQTLENNVVEELFNLSDRLIDQKADIAKNQADIAQNNESIEEL
YDFDNEVAEKIGEIHAYTEEVNKTLQDLITNSVKNTDNIDKNKADID
NNINHIYELAQQQDQHSSDIKTLKNNVEEGLLELSGHLIDQKADLTK
DIKTLENNVEEGLLDLSGRLIDQKADIAKNQADIAQNQTDIQDLAAY
NELQDQYAQKQTEAIDALNKASSENTQNIEDLAAYNELQDAYAKQQT
EAIDALNKASSENTQNIAKNQADIANNINNIYELAQQQDQHSSDIKT
LAKASAANTDRIAKNKADADASFETLTKNQNTLIEKDKEHDKLITAN
KTAIDENKASADTKFAATADAITKNGNAITKNAKSITDLGTKVDGFD
GRVTALDTKVNAFDGRITALDSKVENGMAAQAALSGLFQPYSVGKFN
ATAALGGYGSKSAVAIGAGYRVNPNLAFKAGAAINTSGNKKGSYNIG
VNYEF
SEQ ID NO: 47
UspA2 American P44 (668 amino acids)
MKTMKLLPLKIAVTSALIVGLGTASTANAQVASPANQKIQQKIKKVR
KELRQDIKSLRNDIDSNTADIGSLNDDVADNQDDILDNQADIAKNQD
DIEKNQADIKELDKEVGVLSREIGSLNDDIADNYTDIIDNYTDIIDN
QANIAKNQDDIEKNQADIKELDKEVGVLSREIGSLNDDVADNQDDIA
KNQADIQTLENNVEEGLLELSGHLLDQKADIDNNINNIYELAQQQDQ
HSSDIKTLKKNVEEGLLELSGHLIDQKTDIAQNQANIQDLATYNELQ
DQYAQEQTEAIDALNKASSENTQNIAKNSNRIKALESNVEEGLLELS
GHLIDQKADLTKDIKALESNVEEGLLELSGHLIDQKADIAQNQANIQ
DLAAYNELQDQYAQKQTEAIDALNKASSENTQNIEDLAAYNELQDAY
AKQQTEAIDALNKASSENTQNIAKNQADIANNINNIYELAQQQDQHS
SDIKTLAKASAANTDRIAKNKADADASFETLTKNQNTLIEKDKEHDK
LITANKTAIDANKVSADTKFAATADAITKNGNAITKNAKSITDLGTK
VDAFDSRVTALDTKVNAFDGRITALDSKVENGMAAQAALSGLFQPYS
VGKFNATAALGGYGSKSAVAIGAGYRVNPNLAFKAGAAINTSGNKKG
SYNIGVNYEF
SEQ ID NO: 48
UspA2 American V1171 (674 amino acids)
MKTMKLLPLKIAVTSAMIVGLGATSTVNAQVVEQFFPNIFFNENHDE
LDDAYHNMILGDTAIVSNSQDNSTQLKFYSNDEDSVPDSLLFSKLLH
EQQLNGFKAGDTIIPLDKDGKPVYTKDTRTKDGKVETVYSVTTKIAT
QDDVEQSAYSRGIQGDIDDLYDINREVNEYLKATHDYNERQTEAIDA
LNKASSANTDRIDTAEERIDKNEYDIKALESNVEEGLLELSGHLIDQ
KADLTKDIKALESNVEEGLLELSGHLIDQKADLTKDIKALESNVEEG
LLDLSGRLIDQKADIAQNQANIQDLAAYNELQDAYAKQQTEAIDALN
KASSENTQNIEDLAAYNELQDAYAKQQTEAIDALNKASSENTQNIED
LAAYNELQDAYAKQQTEAIDALNKASSENTQNIEDLAAYNELQDAYA
KQQTEAIDALNKASSENTQNIAKNQADIANNINNIYELAQQQDQHSS
DIKTLAKASAANTDRIAKNKADADASFETLTKNQNTLIEKDKEHDKL
ITANKTAIDANKASADTKFAATADAITKNGNAITKNAKSITDLGTKV
DGFDGRVTALDTKVNALDTKVNAFDGRITALDSKVENGMAAQAALSG
LFQPYSVGKFNATAALGGYGSKSAVAIGAGYRVNPNLAFKAGAAINT
SGNKKGSYNIGVNYEF
SEQ ID NO: 49
UspA2 American TTA24 (613 amino acids)
MKTMKLLPLKIAVTSAMIIGLGAASTANAQSRDRSLEDIQDSISKLV
QDDIDTLKQDQQKMNKYLLLNQLANTLITDELNNNVIKNTNSIEALG
DEIGWLENDIADLEEGVEELTKNQNTLIEKDEEHDRLIAQNQADIQT
LENNVVEELFNLSGRLIDQEADIAKNNASIEELYDFDNEVAERIGEI
HAYTEEVNKTLENLITNSVKNTDNIDKNKADIDNNINHIYELAQQQD
QHSSDIKTLKNNVEEGLLELSGHLIDQKADLTKDIKALESNVEEGLL
DLSGRLLDQKADLTKDIKALESNVEEGLLDLSGRLLDQKADIAQNQT
DIQDLAAYNELQDQYAQKQTEAIDALNKASSENTQNIEDLAAYNELQ
DAYAKQQTEAIDALNKASSENTQNIAKNQADIANNINNIYELAQQQD
QHSSDIKTLAKASAANTNRIATAELGIAENKKDAQIAKAQANANKTA
IDENKASADTKFAATADAITKNGNAITKNAKSITDLGTKVDGFDGRV
TALDTKVNAFDGRITALDSKVENGMAAQAALSGLFQPYSVGKFNATA
ALGGYGSKSAVAIGAGYRVNPNLAFKAGAAINTSGNKKGSYNIGVNY
EF
SEQ ID NO: 50
UspA2 American O35E (576 amino acids)
MKTMKLLPLKIAVTSAMIVGLGATSTVNAQVVEQFFPNIFFNENHDE
LDDAYHNMILGDTAIVSNSQDNSTQLKFYSNDEDSVPDSLLFSKLLH
EQQLNGFKAGDTIIPLDKDGKPVYTKDTRTKDGKVETVYSVTTKIAT
QDDVEQSAYSRGIQGDIDDLYDINREVNEYLKATHDYNERQTEAIDA
LNKASSANTDRIDTAEERIDKNEYDIKALESNVEEGLLELSGHLIDQ
KADLTKDIKALESNVEEGLLELSGHLIDQKADLTKDIKALESNVEEG
LLDLSGRLLDQKADIAKNQADIAQNQTDIQDLAAYNELQDAYAKQQT
EAIDALNKASSENTQNIAKNQADIANNINNIYELAQQQDQHSSDIKT
LAKASAANTDRIAKNKADADASFETLTKNQNTLIEKDKEHDKLITAN
KTAIDANKASADTKFAATADAITKNGNAITKNAKSITDLGTKVDGFD
GRVTALDTKVNALDTKVNAFDGRITALDSKVENGMAAQAALSGLFQP
YSVGKFNATAALGGYGSKSAVAIGAGYRVNPNLAFKAGAAINTSGNK
KGSYNIGVNYEF
SEQ ID NO: 51
UspA2 American SP12-6 (684 amino acids)
MKTMKLLPLKIAVTSAMMVGLGMASTANAQQQKSPKTEIFLPNLFDN
DNTELTDPLYHNMILGNTALLTQENQYKFYADDGNGVPDSLLFNKIL
HDQLLHGFKEGDTIIPLDENGKPVYKLDSIVEQGKTKTVYSVTTKTA
TADDVNSAYSRGIQGDIDDLYEANKENVNRLIEHGDKIFANEESVQY
LNREVQNNIENIHELAQQQDQHSSDIKTLKKNVEKDLLDLSGRLIAQ
KEDIAQNQTDIQDLATYNELQDQYAQKQTEAIDALNKASSENTQNIA
KNSNHIKTLENNIEEGLLELSGHLIDQKADLTKDIKALESNVEEGLL
DLSGRLIDQKADIAQNQANIQDLAAYNELQDAYAKQQTEAIDALNKA
SSENTQNIEDLAAYNELQDAYAKQQTEAIDALNKASSENTQNIEDLA
AYNELQDAYAKQQTEAIDALNKASSENTQNIAKNQADIANNINNIYE
LAQQQDQHSSDIKTLAKASAANTDRIAKNKADADASFETLTKNQNTL
IEKDKEHDKLITANKTAIDANKASADTKFAATADAITKNGNAITKNA
KSITDLGTKVDGFDGRVTALDTKVNALDTKVNAFDGRITALDSKVEN
GMAAQAALSGLFQPYSVGKFNATAALGGYGSKSAVAIGAGYRVNPNL
AFKAGAAINTSGNKKGSYNIGVNYEF
SEQ ID NO: 52
UspA2 American SP12-5 (686 amino acids)
MKTMKLLPLKIAVTSAMIIGLGAASTANAQATETFLPNLFDNDYTET
TDPLYHGMILGNTAITQDTQYKFYAENGNEVPDSLFFNKILHDQQLN
GFKEGDTIIPLDENGKPVYKLDEITENGVKRKVYSVTTKTATREDVE
QSAYSRGIQGDIDDLYEANKENVNRLIEHGDKIFANEESVQYLNKEV
QNNIENIHELAQQQDQHSSDIKTLKKNVEEGLLELSGRLIAQKEDIA
QNQTDIQDLATYNELQDQYAQKQTEAIDALNKASSENTQNIAKNSNH
IKTLENNIEEGLLELSGHLIDQKADLTKDIKALESNVEEGLLDLSGR
LLDQKADIAKNQADIAQNQTDIQDLAAYNELQDQYAQKQTEAIDALN
KASSENTQNIEDLAAYNELQDAYAKQQTEAIDALNKASSENTQNIED
LAAYNELQDAYAKQQTEAIDALNKASSENTQNIAKNQADIANNINNI
YELAQQQDQHSSDIKTLAKASAANTDRIAKNKADADASFETLTKNQN
TLIEKDKEHDKLITANKTAIDANKASADTKFAATADAITKNGNAITK
NAKSITDLGTKVDGFDGRVTALDTKVNALDTKVNAFDGRITALDSKV
ENGMAAQAALSGLFQPYSVGKFNATAALGGYGSKSAVAIGAGYRVNP
NLAFKAGAAINTSGNKKGSYNIGVNYEF
SEQ ID NO: 53
UspA2 Swedish BC5 (630 amino acids)
MKTMKLLPLKIAVTSAMIIGLGAASTANAQAKNDITLEDLPYLIKKI
DQNELEADIGDITALEKYLALSQYGNILALEELNKALEELDEDVGWN
QNDIANLEDDVETLTKNQNALAEQGEAIKEDLQGLADFVEGQEGKIL
QNETSIKKNTQRNLVNGFEIEKNKDAIAKNNESIEDLYDFGHEVAES
IGEIHAHNEAQNETLKGLITNSIENTNNITKNKADIQALENNVVEEL
FNLSGRLIDQKADIDNNINNIYELAQQQDQHSSDIKTLKKNVEEGLL
ELSGHLIDQKTDIAQNQANIQDLATYNELQDQYAQKQTEAIDALNKA
SSENTQNIEDLAAYNELQDAYAKQQTEAIDALNKASSENTQNIEDLA
AYNELQDAYAKQQTEAIDALNKASSENTQNIAKNQADIANNINNIYE
LAQQQDQHSSDIKTLAKASAANTDRIAKNKADADASFETLTKNQNTL
IEKDKEHDKLITANKTAIDANKASADTKFAATADAITKNGNAITKNA
KSITDLGTKVDGFDSRVTALDTKVNAFDGRITALDSKVENGMAAQAA
LSGLFQPYSVGKFNATAALGGYGSKSAVAIGAGYRVNPNLAFKAGAA
INTSGNKKGSYNIGVNYEF
SEQ ID NO: 54
UspA2 American 7169 (616 amino acids)
MKTMKLLPLKIAVTSALIVGLGAASTANAQAQDRSLEQIQDKLANLV
EKIEQAKSQNGQSQKDINQYLLLSQYANVLTMEELNNNVVKNSSSIE
TLDNDIAWLNDDLIDLDKEVGVLSRDIGSLHDDVAQNQADIKTLKNN
VVEELFNLSDRLIDQEADIAQNNESIEDLYDFGREVAESIGEIHAHN
EAQNETLKDLITNSVKNTDNITKNKADIQALENDVGKELLNLSGRLI
DQKADIDNNINHIYELAQQQDQHSSDIKTLKNNVEEGLLELSGHLID
QKADLTKDIKALESNVEEGLLDLSGRLLDQKADIAQNQANIQDLAAY
NELQDAYAKQQTEAIDALNKASSENTQNIEDLAAYNELQDAYAKQQT
EAIDALNKASSENTQNIAKNQADIANNINNIYELAQQQDQHSSDIKT
LAKASAANTDRIAKNKADADASFETLTKNQNTLIEKDKEHDKLITAN
KTAIDANKASADTKFAATADAITKNGNAITKNAKSITDLGTKVDGFD
SRVTALDTKVNAFDGRITALDSKVENGMAAQAALSGLFQPYSVGKFN
ATAALGGYGSKSAVAIGAGYRVNPNLAFKAGAAINTSGNKKGSYNIG
VNYEF
SEQ ID NO: 55
UspA2 Finnish FIN2344 (614 amino acids)
MKTMKLLPLKIAVTSAMIIGLGATSTVNAQVVEQFFPNIFFNENHDE
LDDAYHNMILGDTAIVSNSQDNSTQLKFYSNDEDSVPDSLLFSKLLH
EQQLNGFKAGDTIIPLDKDGKPVYTKDTRTKDGKVETVYSVTTKIAT
QDDVEQSAYSRGIQGDIDDLYDINREVNEYLKATHDYNERQTEAIDA
LNKASSANTDRIDTAEERIDKNEYDIKALESNVGKDLLDLSGRLIAQ
KEDIDNNINHIYELAQQQDQHSSDIKTLKNNVEEGLLELSGHLIDQK
ADLTKDIKTLESNVEEGLLDLSGRLIDQKADIAQNQANIQDLAAYNE
LQDQYAQKQTEAIDALNKASSENTQNIEDLAAYNELQDAYAKQQTEA
IDALNKASSENTQNIAKNQADIANNINNIYELAQQQDQHSSDIKTLA
KVSAANTDRIAKNKADADASFETLTKNQNTLIEKDKEHDKLITANKT
AIDANKASADTKFAATADAITKNGNAITKNAKSITDLGTKVDGFDGR
VTALDTKVNAFDGRITALDSKVENGMAAQAALSGLFQPYSVGKFNAT
AALGGYGSKSAVAIGAGYRVNPNLAFKAGAAINTSGNKKGSYNIGVN
YEF
SEQ ID NO: 56
UspA2 American V1118 (679 amino acids)
MKTMKLPPLKIAVTSAMIIGLGAASTANAQTTETFLPNLFDNDYTET
TDPLYHGMILGDTAITQDTQYKFYAENGNEVPDSLFFNKILHDQLLN
GFKAGDTIIPLDENGKPVYKLDERTENGVKRKVYSVTTKTATQADVE
QSAYSRGIQGDIDDLYEANKENVNRLIEHGDKIFANEESVQYLNREV
QNNIENIHELAQQQDQHSSDIKTLKKNVEKDLLDLSGRLIAQKEDIA
QNQTDIQDLATYNELQDQYAQKQTEAIDALNKASSENTQNIAKNSNH
IKTLENNIEECLLELSGHLIDQKADLTKDIKALESNVEEGLLDLSGR
LIDQKADIAQNQANIQDLAAYNELQDAYAKQQTEAIDALNKASSENT
QNIEDLAAYNELQDAYAKQQTEAIDALNKASSENTQNIEDLAAYNEL
QDAYAKQQTEAIDALNKASSENTQNIAKNQADIANNINNIYELAQQQ
DQHSSDIKTLAKASAANTDRIAKNKADADASFETLTKNQNTLIEKDK
EHDKLITANKTAIDANKASADTKFAATADAITKNGNAITKNAKSITD
LGTKVDGFDGRVTALDTKVNALDTKVNAFDGRITALDSKVENGMAAQ
AALSGLFQPYSVGKFNATAALGGYGSKSAVAIGAGYRVNPNLAFKAG
AAINTSGNKKGSYNIGVNYEF
SEQ ID NO: 57
UspA2 American V1145 (724 amino acids)
MKTMKLLPLKIAVTSALIVGLGAASTANAQETLEEVLESIKQINEQD
LQDDIGYNSALDRYLVLSQYGNLLIAKELNENVEKNSNSIAKNSNSI
ADLEADVGYLAENQNTLIEQNETINQELEGITHELESFIAYAHAQDQ
KNLVNEFEIEKNKDAIAKNNESIEDLYDFGHEVAESIGEIHAYTEEV
NKTLENLITNSVKNTDNITKNKADIQALESNVEKELLNLSGRLIDQK
ADIDNNINHIYELAQQQDQHSSDIKTLKKNVEEGLLELSGHLIDQKS
DIAQNQTDIQDLATYNELQDQYAQKQTEAIDALNKASSENTQNIEDL
AAYNELQDAYAKQQTEAIDALNKASSENTQNIEDLAAYNELQDAYAK
QQTEAIDALNKASSENTQNIEDLAAYNELQDAYAKQQTEAIDALNKA
SSENTQNIEDLAAYNELQDAYAKQQTEAIDALNKASSENTQNIEDLA
AYNELQDAYAKQQTEAIDALNKASSENTQNIAKNQADIANNINNIYE
LAQQQDQHSSDIKTLAKASAANTDRIAKNKADADASFETLTKNQNTL
IEKDKEHDKLITANKTAIDANKASADTKFAATADAITKNGNAITKNA
KSITDLGTKVDGFDSRVTALDTKVNAFDGRITALDSKVENGMAAQAA
LSGLFQPYSVGKFNATAALGGYGSKSAVAIGAGYRVNPNLAFKAGAA
INTSGNKKGSYNIGVNYEF
SEQ ID NO: 58
UspA2 American V1156 (611 amino acids)
MKTMKLLPLKIAVTSALIVGLGAASTANAQAQARDRSLEDIQALIGN
IDVDKIRSQKQKNPEIFQYLLLNQLSNTLITDELNNNVIKNTNSIET
LDNDIAWLNDDLIDLDKEVGVLSRDIGSLHDDVAQNQADIKTLENNV
VEELFNLSDRLIDQEAEIAQNNESIEDLYDFGREVAESIGEIHAHNE
AQNETLKDLITNSVKNTDNIDKNKADIQALENNVEEGLLELSGHLID
QKADLTKDIKALESNVEEGLLDLSGRLLDQKADIAKNQADIAQNQTD
IQDLAAYNELQDQYAQKQTEAIDALNKASSENTQNIEDLAAYNELQD
AYAKQQTEAIDALNKASSENTQNIEDLAAYNELQDAYAKQQTEAIDA
LNKASSENTQNIAKNQADIANNINNIYELAQQQDQHSSDIKTLAKVS
AANTDRIAKNKADADASFETLTKNQNTLIEKDKEHDKLITANKTAID
ANKASADTKFAATADAITKNGNAITKNAKSITDLGTKVDGFDSRVTA
LDTKVNAFDGRITALDSKVENGMAAQAALSGLFQPYSVGKFNATAAL
GGYGSKSAVAIGAGYRVNPNLAFKAGAAINTSGNKKGSYNIGVNYEF

1. An immunogenic composition comprising: (i) an immunogenic polypeptide from non-typeable Haemophilus influenzae, (ii) a PE-PilA fusion protein, and (iii) an adjuvant, for reducing the frequency of severe exacerbations, in a subject having chronic obstructive pulmonary disease (COPD).

2. (canceled)

3. A method of reducing the frequency of severe exacerbations, in a subject having chronic obstructive pulmonary disease (COPD), said method comprising administering to said subject a therapeutically effective amount of an immunogenic composition comprising: (i) an immunogenic polypeptide from non-typeable Haemophilus influenzae, (ii) a PE-PilA fusion protein, and (iii) an adjuvant.

4. The method of claim 3, wherein the yearly rate of severe exacerbations is reduced in the subject who has been administered the immunogenic composition as compared to a subject who has not been administered the immunogenic composition.

5. The method of claim 3, wherein the frequency of pneumonia due to COPD is reduced in a subject having chronic obstructive pulmonary disease (COPD).

6. The method of claim 3, wherein the subject has GOLD 4 (very severe) COPD status.

7. The method of claim 3, further comprising: (a) selecting a subject who has GOLD 4 (very severe) COPD status, and (b) administering to the subject the immunogenic composition.

8. The method of claim 3, wherein the subject has experienced at least 2 moderate AECOPD or at least one severe AECOPD in the previous 12 months.

9. The method of claim 3, wherein the subject is taking ICS.

10. The method of claim 3, wherein the subject has experienced at least 2 moderate AECOPD or at least one severe AECOPD in the previous 12 months and is taking ICS.

11. The method of claim 3, wherein the subject has previously been administered a pneumococcal vaccine and/or has been administered an influenza vaccine in the previous 12 months.

12. The method of claim 3, wherein the subject has experienced at least one severe AECOPD in the previous 12 months.

13. The immunogenic composition of claim 1, wherein the immunogenic polypeptide from non-typeable Haemophilus influenzae is an immunogenic polypeptide of Protein D.

14. The immunogenic composition of claim 1, wherein the PE-PilA fusion protein is an isolated immunogenic polypeptide with at least 70%, 80%, 85%, 90%, 91%, 92%, 93%, 94%, 95%, 96%, 97%, 98%, 99%, or 100% identity to SEQ ID NO: 9.

15. (canceled)

16. The immunogenic composition of claim 12, wherein the immunogenic polypeptide from non-typeable Haemophilus influenzae is an isolated immunogenic polypeptide with at least 70%, 80%, 85%, 90%, 91%, 92%, 93%, 94%, 95%, 96%, 97%, 98%, 99%, or 100% identity to SEQ ID NO: 2.

17. The immunogenic composition of claim 1, further comprising an immunogenic polypeptide of UspA2 from Moraxella catarrhal's.

18. The immunogenic composition of claim 17, wherein the immunogenic polypeptide of UspA2 from Moraxella catarrhalis is an isolated immunogenic polypeptide with at least 70%, 80%, 85%, 90%, 91%, 92%, 93%, 94%, 95%, 96%, 97%, 98%, 99%, or 100% identity to a polypeptide selected from the group consisting of MC-001 (SEQ ID NO: 11), MC-002 (SEQ ID NO: 12), MC-003 (SEQ ID NO: 13), MC-004 (SEQ ID NO: 14), MC-005 (SEQ ID NO: 15), MC-006 (SEQ ID NO: 16), MC-007 (SEQ ID NO: 17), MC-008 (SEQ ID NO:18), MC-009 (SEQ ID NO: 19), MC-010 (SEQ ID NO: 20) or MC-011 (SEQ ID NO: 21).

19. The immunogenic composition of claim 18, wherein the polypeptide is MC-009 (SEQ ID NO: 19).

20. The immunogenic composition of claim 1, wherein the subject is a human.

21. The method of claim 3, wherein the frequency of pneumonia due to AECOPD is reduced in a subject having chronic obstructive pulmonary disease (COPD).

22. The method of claim 3, further comprising an immunogenic polypeptide of UspA2 from Moraxella catarrhal's.

23. The method of claim 22, wherein the immunogenic polypeptide of UspA2 from Moraxella catarrhalis is an isolated immunogenic polypeptide with at least 70%, 80%, 85%, 90%, 91%, 92%, 93%, 94%, 95%, 96%, 97%, 98%, 99%, or 100% identity to a polypeptide selected from the group consisting of MC-001 (SEQ ID NO: 11), MC-002 (SEQ ID NO: 12), MC-003 (SEQ ID NO: 13), MC-004 (SEQ ID NO: 14), MC-005 (SEQ ID NO: 15), MC-006 (SEQ ID NO: 16), MC-007 (SEQ ID NO: 17), MC-008 (SEQ ID NO:18), MC-009 (SEQ ID NO: 19), MC-010 (SEQ ID NO: 20) or MC-011 (SEQ ID NO: 21).

24. The method of claim 23, wherein the polypeptide is MC-009 (SEQ ID NO: 19).

25. The method of claim 3, wherein the subject is a human.


SEQUENCE LISTING (58 sequences)

SEQ ID NO: 1 - protein, 364 aa, H. influenzae
MKLKTLALSLLAAGVLAGCSSHSSNMANTQMKSDKIIIAHRGASGYLPEHTLESKALAFAQQADYLEQDLAMTKDGRLVVIHDHFLDGLTDVAKKFPHRHRKDGRYYVIDFTLKEIQSLEMTENFETKDGKQAQVYPNRFPLWKSHFRIHTFEDEIEFIQGLEKSTGKKVGIYPEIKAPWFHHQNGKDIAAETLKVLKKYGYDKKTDMVYLQTFDFNELKRIKTELLPQMGMDLKLVQLIAYTDWKETQEKDPKGYWVNYNYDWMFKPGAMAEVVKYADGVGPGWYMLVNKEESKPDNIVYTPLVKELAQYNVEVHPYTVRKDALPEFFTDVNQMYDALLNKSGATGVFTDFPDTGVEFLKGIK

SEQ ID NO: 2 - protein, 348 aa, Artificial
  Protein D fragment with MDP tripeptide from NS1
MDPSSHSSNMANTQMKSDKIIIAHRGASGYLPEHTLESKALAFAQQADYLEQDLAMTKDGRLVVIHDHFLDGLTDVAKKFPHRHRKDGRYYVIDFTLKEIQSLEMTENFETKDGKQAQVYPNRFPLWKSHFRIHTFEDEIEFIQGLEKSTGKKVGIYPEIKAPWFHHQNGKDIAAETLKVLKKYGYDKKTDMVYLQTFDFNELKRIKTELLPQMGMDLKLVQLIAYTDWKETQEKDPKGYWVNYNYDWMFKPGAMAEVVKYADGVGPGWYMLVNKEESKPDNIVYTPLVKELAQYNVEVHPYTVRKDALPEFFTDVNQMYDALLNKSGATGVFTDFPDTGVEFLKGIK

SEQ ID NO: 3 - protein, 10 aa, H. influenzae
SSHSSNMANT

SEQ ID NO: 4 - protein, 160 aa, H. influenzae
MKKIILTLSLGLLTACSAQIQKAEQNDVKLAPPTDVRSGYIRLVKNVNYYIDSESIWVDNQEPQIVHFDAVVNLDKGLYVYPEPKRYARSVRQYKILNCANYHLTQVRTDFYDEFWGQGLRAAPKKQKKHTLSLTPDTTLYNAAQIICANYGEAFSVDKK

SEQ ID NO: 5 - protein, 141 aa, H. influenzae
IQKAEQNDVKLAPPTDVRSGYIRLVKNVNYYIDSESIWVDNQEPQIVHFDAVVNLDKGLYVYPEPKRYARSVRQYKILNCANYHLTQVRTDFYDEFWGQGLRAAPKKQKKHTLSLTPDTTLYNAAQIICANYGEAFSVDKK

SEQ ID NO: 6 - protein, 149 aa, H. influenzae
MKLTTQQTLKKGFTLIELMIVIAIIAILATIAIPSYQNYTKKAAVSELLQASAPYKADVELCVYSTNETTNCTGGKNGIAADITTAKGYVKSVTTSNGAITVKGDGTLANMEYILQATGNAATGVTWTTTCKGTDASLFPANFCGSVTQ

SEQ ID NO: 7 - protein, 110 aa, H. influenzae
TKKAAVSELLQASAPYKADVELCVYSTNETTNCTGGKNGIAADITTAKGYVKSVTTSNGAITVKGDGTLANMEYILQATGNAATGVTWTTTCKGTDASLFPANFCGSVTQ

SEQ ID NO: 8 - protein, 275 aa, Artificial
  LVL735 (protein) (pelB sp)(ProtE aa 20-160)(GG)(PilA aa40-149)
MKYLLPTAAAGLLLLAAQPAMAIQKAEQNDVKLAPPTDVRSGYIRLVKNVNYYIDSESIWVDNQEPQIVHFDAVVNLDKGLYVYPEPKRYARSVRQYKILNCANYHLTQVRTDFYDEFWGQGLRAAPKKQKKHTLSLTPDTTLYNAAQIICANYGEAFSVDKKGGTKKAAVSELLQASAPYKADVELCVYSTNETTNCTGGKNGIAADITTAKGYVKSVTTSNGAITVKGDGTLANMEYILQATGNAATGVTWTTTCKGTDASLFPANFCGSVTQ

SEQ ID NO: 9 - protein, 253 aa, Artificial
  PE-PilA fusion protein without signal peptide
IQKAEQNDVKLAPPTDVRSGYIRLVKNVNYYIDSESIWVDNQEPQIVHFDAVVNLDKGLYVYPEPKRYARSVRQYKILNCANYHLTQVRTDFYDEFWGQGLRAAPKKQKKHTLSLTPDTTLYNAAQIICANYGEAFSVDKKGGTKKAAVSELLQASAPYKADVELCVYSTNETTNCTGGKNGIAADITTAKGYVKSVTTSNGAITVKGDGTLANMEYILQATGNAATGVTWTTTCKGTDASLFPANFCGSVTQ

SEQ ID NO: 10 - protein, 630 aa, Moraxalla catarrhalis
MKTMKLLPLKIAVTSAMIIGLGAASTANAQAKNDITLEDLPYLIKKIDQNELEADIGDITALEKYLALSQYGNILALEELNKALEELDEDVGWNQNDIANLEDDVETLTKNQNALAEQGEAIKEDLQGLADFVEGQEGKILQNETSIKKNTQRNLVNGFEIEKNKDAIAKNNESIEDLYDFGHEVAESIGEIHAHNEAQNETLKGLITNSIENTNNITKNKADIQALENNVVEELFNLSGRLIDQKADIDNNINNIYELAQQQDQHSSDIKTLKKNVEEGLLELSGHLIDQKTDIAQNQANIQDLATYNELQDQYAQKQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDSRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 11 - protein, 520 aa, Artificial
  MC-001 (protein) - (M)(UspA2 amino acids 30 - 540)(ASHHHHHH)
MQAKNDITLEDLPYLIKKIDQNELEADIGDITALEKYLALSQYGNILALEELNKALEELDEDVGWNQNDIANLEDDVETLTKNQNALAEQGEAIKEDLQGLADFVEGQEGKILQNETSIKKNTQRNLVNGFEIEKNKDAIAKNNESIEDLYDFGHEVAESIGEIHAHNEAQNETLKGLITNSIENTNNITKNKADIQALENNVVEELFNLSGRLIDQKADIDNNINNIYELAQQQDQHSSDIKTLKKNVEEGLLELSGHLIDQKTDIAQNQANIQDLATYNELQDQYAQKQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDSRVTALDTKASHHHHHH

SEQ ID NO: 12 - protein, 512 aa, Moraxalla catarrhalis
MQAKNDITLEDLPYLIKKIDQNELEADIGDITALEKYLALSQYGNILALEELNKALEELDEDVGWNQNDIANLEDDVETLTKNQNALAEQGEAIKEDLQGLADFVEGQEGKILQNETSIKKNTQRNLVNGFEIEKNKDAIAKNNESIEDLYDFGHEVAESIGEIHAHNEAQNETLKGLITNSIENTNNITKNKADIQALENNVVEELFNLSGRLIDQKADIDNNINNIYELAQQQDQHSSDIKTLKKNVEEGLLELSGHLIDQKTDIAQNQANIQDLATYNELQDQYAQKQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDSRVTALDTK

SEQ ID NO: 13 - protein, 513 aa, Artificial
  MC-003 (Protein) - (M)(UspA2 amino acids 30-540)(H)
MQAKNDITLEDLPYLIKKIDQNELEADIGDITALEKYLALSQYGNILALEELNKALEELDEDVGWNQNDIANLEDDVETLTKNQNALAEQGEAIKEDLQGLADFVEGQEGKILQNETSIKKNTQRNLVNGFEIEKNKDAIAKNNESIEDLYDFGHEVAESIGEIHAHNEAQNETLKGLITNSIENTNNITKNKADIQALENNVVEELFNLSGRLIDQKADIDNNINNIYELAQQQDQHSSDIKTLKKNVEEGLLELSGHLIDQKTDIAQNQANIQDLATYNELQDQYAQKQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDSRVTALDTKH

SEQ ID NO: 14 - protein, 514 aa, Artificial
  MC-004 (Protein) - (M)(UspA2 amino acids 30-540)(HH)
MQAKNDITLEDLPYLIKKIDQNELEADIGDITALEKYLALSQYGNILALEELNKALEELDEDVGWNQNDIANLEDDVETLTKNQNALAEQGEAIKEDLQGLADFVEGQEGKILQNETSIKKNTQRNLVNGFEIEKNKDAIAKNNESIEDLYDFGHEVAESIGEIHAHNEAQNETLKGLITNSIENTNNITKNKADIQALENNVVEELFNLSGRLIDQKADIDNNINNIYELAQQQDQHSSDIKTLKKNVEEGLLELSGHLIDQKTDIAQNQANIQDLATYNELQDQYAQKQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDSRVTALDTKHH

SEQ ID NO: 15 - protein, 499 aa, Artificial
  MC-005 (Protein) - (M)(UspA2 amino acids 30-519)(ASHHHHHH)
MQAKNDITLEDLPYLIKKIDQNELEADIGDITALEKYLALSQYGNILALEELNKALEELDEDVGWNQNDIANLEDDVETLTKNQNALAEQGEAIKEDLQGLADFVEGQEGKILQNETSIKKNTQRNLVNGFEIEKNKDAIAKNNESIEDLYDFGHEVAESIGEIHAHNEAQNETLKGLITNSIENTNNITKNKADIQALENNVVEELFNLSGRLIDQKADIDNNINNIYELAQQQDQHSSDIKTLKKNVEEGLLELSGHLIDQKTDIAQNQANIQDLATYNELQDQYAQKQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSASHHHHHH

SEQ ID NO: 16 - protein, 491 aa, Moraxalla catarrhalis
MQAKNDITLEDLPYLIKKIDQNELEADIGDITALEKYLALSQYGNILALEELNKALEELDEDVGWNQNDIANLEDDVETLTKNQNALAEQGEAIKEDLQGLADFVEGQEGKILQNETSIKKNTQRNLVNGFEIEKNKDAIAKNNESIEDLYDFGHEVAESIGEIHAHNEAQNETLKGLITNSIENTNNITKNKADIQALENNVVEELFNLSGRLIDQKADIDNNINNIYELAQQQDQHSSDIKTLKKNVEEGLLELSGHLIDQKTDIAQNQANIQDLATYNELQDQYAQKQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKS

SEQ ID NO: 17 - protein, 544 aa, Artificial
  MC-007 (Protein) - (M)(UspA2 amino acids 30-564)(ASHHHHHH)
MQAKNDITLEDLPYLIKKIDQNELEADIGDITALEKYLALSQYGNILALEELNKALEELDEDVGWNQNDIANLEDDVETLTKNQNALAEQGEAIKEDLQGLADFVEGQEGKILQNETSIKKNTQRNLVNGFEIEKNKDAIAKNNESIEDLYDFGHEVAESIGEIHAHNEAQNETLKGLITNSIENTNNITKNKADIQALENNVVEELFNLSGRLIDQKADIDNNINNIYELAQQQDQHSSDIKTLKKNVEEGLLELSGHLIDQKTDIAQNQANIQDLATYNELQDQYAQKQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDSRVTALDTKVNAFDGRITALDSKVENGMAAQAAASHHHHHH

SEQ ID NO: 18 - protein, 538 aa, Artificial
  MC-008 (Protein) - (M)(UspA2 30-564)(HH)
MQAKNDITLEDLPYLIKKIDQNELEADIGDITALEKYLALSQYGNILALEELNKALEELDEDVGWNQNDIANLEDDVETLTKNQNALAEQGEAIKEDLQGLADFVEGQEGKILQNETSIKKNTQRNLVNGFEIEKNKDAIAKNNESIEDLYDFGHEVAESIGEIHAHNEAQNETLKGLITNSIENTNNITKNKADIQALENNVVEELFNLSGRLIDQKADIDNNINNIYELAQQQDQHSSDIKTLKKNVEEGLLELSGHLIDQKTDIAQNQANIQDLATYNELQDQYAQKQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDSRVTALDTKVNAFDGRITALDSKVENGMAAQAAHH

SEQ ID NO: 19 - protein, 537 aa, Artificial
  MC-009 (Protein) - (M)(UspA2 31-564)(HH)
MAKNDITLEDLPYLIKKIDQNELEADIGDITALEKYLALSQYGNILALEELNKALEELDEDVGWNQNDIANLEDDVETLTKNQNALAEQGEAIKEDLQGLADFVEGQEGKILQNETSIKKNTQRNLVNGFEIEKNKDAIAKNNESIEDLYDFGHEVAESIGEIHAHNEAQNETLKGLITNSIENTNNITKNKADIQALENNVVEELFNLSGRLIDQKADIDNNINNIYELAQQQDQHSSDIKTLKKNVEEGLLELSGHLIDQKTDIAQNQANIQDLATYNELQDQYAQKQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDSRVTALDTKVNAFDGRITALDSKVENGMAAQAAHH

SEQ ID NO: 20 - protein, 536 aa, Moraxalla catarrhalis
MQAKNDITLEDLPYLIKKIDQNELEADIGDITALEKYLALSQYGNILALEELNKALEELDEDVGWNQNDIANLEDDVETLTKNQNALAEQGEAIKEDLQGLADFVEGQEGKILQNETSIKKNTQRNLVNGFEIEKNKDAIAKNNESIEDLYDFGHEVAESIGEIHAHNEAQNETLKGLITNSIENTNNITKNKADIQALENNVVEELFNLSGRLIDQKADIDNNINNIYELAQQQDQHSSDIKTLKKNVEEGLLELSGHLIDQKTDIAQNQANIQDLATYNELQDQYAQKQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDSRVTALDTKVNAFDGRITALDSKVENGMAAQAA

SEQ ID NO: 21 - protein, 519 aa, Artificial
  MC-011 (Protein) - (M)(UspA2 amino acids 31-540)(ASHHHHHH)
MAKNDITLEDLPYLIKKIDQNELEADIGDITALEKYLALSQYGNILALEELNKALEELDEDVGWNQNDIANLEDDVETLTKNQNALAEQGEAIKEDLQGLADFVEGQEGKILQNETSIKKNTQRNLVNGFEIEKNKDAIAKNNESIEDLYDFGHEVAESIGEIHAHNEAQNETLKGLITNSIENTNNITKNKADIQALENNVVEELFNLSGRLIDQKADIDNNINNIYELAQQQDQHSSDIKTLKKNVEEGLLELSGHLIDQKTDIAQNQANIQDLATYNELQDQYAQKQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDSRVTALDTKASHHHHHH

SEQ ID NO: 22 - protein, 613 aa, Moraxalla catarrhalis
MKTMKLLPLKIAVTSAMIIGLGAASTANAQSRDRSLEDIQDSISKLVQDDINTLKQDQQKMNKYLLLNQLANTLITDELNNNVIKNTNSIEALGDEIGWLENDIADLEEGVEELTKNQNTLIEKDEEHDRLIAQNQADIQTLENNVVEELFNLSGRLIDQEADIAKNNASIEELYDFDNEVAERIGEIHAYTEEVNKTLENLITNSVKNTDNIDKNKADIDNNINHIYELAQQQDQHSSDIKTLKNNVEEGLLELSGHLIDQKADLTKDIKALESNVEEGLLDLSGRLLDQKADLTKDIKALESNVEEGLLDLSGRLLDQKADIAQNQTDIQDLAAYNELQDQYAQKQTEAIDALNKASSENTQNIEDLAAYNELQDAYAKQQTEAIDALNKASSENTQNIAKNQADIANNINNIYELAQQQDQHSSDIKTLAKASAANTNRIATAELGIAENKKDAQIAKAQANANKTAIDENKASADTKFAATADAITKNGNAITKNAKSITDLGTKVDGFDGRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 23 - protein, 644 aa, Moraxalla catarrhalis
MKTMKLLPLKIAVTSALIIGLGAASTANAQQQLQTETFLPNFLSNDNYDLTDPFYHNMILGDTALLDKQDGSQPQLKFYSNDKDSVPDSLLFSKLLHEQQLNGFKKGDTIIPLDKDGKPVYQVDYKLDGKGKKQKRRQVYSVTTKTATDDDVNSAYSRGILGKVDDLDDEMNFLNHDITSLYDVTANQQDAIKDLKKGVKGLNKELKELDKEVGVLSRDIGSLNDDVAQNNESIEDLYDFSQEVADSIGEIHAHNKAQNETLQDLITNSVENTNNITKNKADIQALENNVVEELFNLSGRLIDQKADLTKDIKTLESNVEEGLLELSGHLIDQKADIAKNQADIAQNQANIQDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDENKASADTKFAATADAITKNGNAITKNAKSITDLGTKVDGFDSRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 24 - protein, 591 aa, Moraxalla catarrhalis
MKTMKLLPLKIAVTSALIVGLGAASTANAQLVERFFPNIFLDKPLAKQHYHNVVVGDTSIVSDLQSNSDQLKFYSDDEGLVPDSLLFNKMLHEQLLNGFKEGDTIIPLDENGKPVYKVDYKLDGKEPRKVYSVTTKIATAEDVATSSYANGIQKDIDDLYDFDHQVTERLTQHGKTIYRNGERILANEESVQYLNKEVQNNIEHIYELAQQQDQHSSDIKTLESNVEKGLLELSGHLIDQKADLTKDIKTLESNVEEGLLDLSGRLIDQKADLTKDIKTLESNVEEGLLDLSGRLIDQKADIAQNQANIQDLAAYNELQDQYAQKQTEAIDALNKASSENTQNIEDLAAYNELQDAYAKQQTEAIDALNKASSENTQNIAKNQADIANNINNIYELAQQQDQHSSDIKTLAKASAANTNRIATAELGIAENKKDAQIAKAQANANKTAIDENKASADTKFAATADAITKNGNAITKNAKSITDLGTKVDGFDSRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 25 - protein, 687 aa, Moraxalla catarrhalis
MKTMKLLPLKIAVTSAMIIGLGAASTANAQQQQQQQQQQQSRTEIFFPNIFFNENHDELDDAYHNIILGDTALLDKQDGSQPQLKFYSNDKDSVPDSLLFSKLLHEQQLNGFKKGDTIIPLDKDGKPVYQVDYKLDGKGKKQKRRQVYSVTTKTATDDDVNSAYSRGILGKVDDLDDEMNFLNHDITSLYDVTANQQDAIKGLKKGVKGLNKELKELDKEVGVLSRDIGSLNDDVAQNNESIEDLYDFSQEVADSIGEIHAHNKAQNETLQDLITNSVENTNNITKNKADIQALENNVVEELFNLSGRLIDQKADLTKDIKTLESNVEEGLLELSGHLIDQKADIAKNQADIAQNQANIQDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDENKASADTKFAATADAITKNGNAITKNAKSITDLGTKVDAFDGRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 26 - protein, 683 aa, Moraxalla catarrhalis
MKTMKLLPLKIAVTSAMIVGLGMASTANAQQQKSPKTETFLPNIFFNEYADDLDTLYHNMILGDTAITHDDQYKFYADDATEVPDSLFFNKILHDQLLYGFKEGDKIIPLDENGKPVYKLDKRLENGVQKTVYSVTTKTATADDVNSAYSRGIQGDIDDLYEANKENVNRLIEHGDKIFANEESVQYLNREVQNNIENIHELAQQQDQHSSDIKTLKKNVEKDLLDLSGRLIAQKEDIAQNQTDIQDLATYNELQDQYAQKQTEAIDALNKASSENTQNIAKNSNHIKTLENNIEEGLLELSGHLIDQKADLTKDIKALESNVEEGLLDLSGRLIDQKADIAQNQANIQDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDGRVTALDTKVNALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 27 - protein, 684 aa, Moraxalla catarrhalis
MKTMKLLPLKIAVTSAMMVGLGMASTANAQQQKSPKTEIFLPNLFDNDNTELTDPLYHNMILGNTALLTQENQYKFYADDGNGVPDSLLFNKILHDQLLHGFKEGGTIIPLDENGKPVYKLDSIVEQGKTKTVYSVTTKTATADDVNSAYSRGIQGDIDDLYEANKENVNRLIEHGDKIFANEESVQYLNREVQNNIENIHELAQQQDQHSSDIKTLKKNVEKDLLDLSGRLIAQKEDIAQNQTDIQDLATYNELQDQYAQKQTEAIDALNKASSENTQNIAKNSNHIKTLENNIEEGLLELSGHLIDQKADLTKDIKALESNVEEGLLDLSGRLIDQKADIAQNQANIQDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDGRVTALDTKVNALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 28 - protein, 684 aa, Moraxalla catarrhalis
MKTMKLLPLKIAVTSAMIIGLGAASTANAQQQQKTKTEVFLPNLFDNDYYDLTDPLYHSMILGDTATLFDQQDNSKSQLKFYSNDKDSVPDSLLFSKLLHEQQLNGFKAGDTIIPLDKDGKPVYTQDTRTKDGKVETVYSVTTKIATQDDVEQSAYSRGIQGDIDDLYDINREVNEYLKATHDYNERQTEAIDALNKASSANTDRIDTAEERIDKNEYDIKALESNVGKDLLDLSGRLIAQKEDIDNNINHIYELAQQQDQHSSDIKTLKNNVEEGLLELSGHLIDQKADLTKDIKTLENNIEEGLLELSGHLIDQKADLTKDIKTLENNIEEGLLELSGHLIDQKADIAQNQANIQDLAAYNELQDQYAQKQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKVSAANTDRIAKNKADADASFETLTKNQNTLIEKDKEHDKLITANKTAIDANKASADTKFAATADAITKNGNAITKNAKSITDLGTKVDGFDGRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 29 - protein, 684 aa, Moraxalla catarrhalis
MKTMKLLPLKIAVTSAMMVGLGMASTANAQQQKSPKTEIFLPNLFDNDNTELTDPLYHNMILGNTALLTQENQYKFYADDGNGVPDSLLFNKILHDQLLHGFKKGDTIIPLDENGKPVYKLDSIVEQGKTKTVYSVTTKTATADDVNSAYSRGIQGDIDDLYEANKENVNRLIEHGDKIFANEESVQYLNREVQNNIENIYELVQQQDQHSSDIKTLKKNVEKDLLDLSGRLIAQKEDIAQNQTDIQDLATYNELQDQYAQKQTEAIDALNKASSENTQNIAKNSNHIKTLENNIEEGLLELSGHLIDQKADLTKDIKALESNVEEGLLDLSGRLIDQKADIAQNQANIQDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDGRVTALDTKVNALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 30 - protein, 574 aa, Moraxalla catarrhalis
MKTMKLLPLKIAVTSAMIIGLGAASTANAQLAEQFFPNIFSNHAPVKQHYHNVVVGDTSIVENLQDSDDTQLKFYSNDEYSVPDSLLFNKMLHEQQLNGFKKGDTIIPLDENGKPVYKVDYKLDGQEPRRVYSVTTKIATQDDVDNSPYSRGIQGDIDDLYEANKENVNRLIEHGDKIFANEESVQYLNKEVQNNIENIYELAQQQDQHSSDIKTLKKNVEEGLLELSGHLIDQKADLTKDIKTLESNVEEGLLELSGHLIDQKADIAKNQADIAQNQANIQDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDAFDGRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 31 - protein, 678 aa, Moraxalla catarrhalis
MKTMKLLPLKIAVTSALIVGLGAASTANAQQQPQTETFFPNIFFNENHDALDDVYHNMILGDTAITQDNQYKFYADAISEVPDSLLFNKILHDQQLNGFKEGDTIIPLDENGKPVYKLDEKVENGVKKSVYSVTTKTATRADVEQSAYSRGIQGDIDDLYEANKENVNRLIEHGDKIFANEESVQYLNKEVQNNIENIHELAQQQDQHSSDIKTLKKNVEEGLLELSGHLIDQKADLTKDIKTLESNVEEGLLDLSGRLLDQKADIAQNQANIQDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDAFDGRVTALDTKVNALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 32 - protein, 678 aa, Moraxalla catarrhalis
MKTMKLLPLKIAVTSAMIVGLGAASTANAQQQQQPRTETFFPNIFFNENHDALDDVYHNMILGDTAITQDNQYKFYADAISEVPDSLLFNKILHDQQLNGFKEGDTIIPLDENGKPVYKLDEKVENGVKKSVYSVTTKTATRADVEQSAYSRGIQGDIDDLYEANKENVNRLIEHGDKIFANEESVQYLNREVQNNIENIHELAQQQDQHSSDIKTLKKNVEKDLLDLSGRLIAQKEDIAQNQTDIQDLATYNELQDQYAQKQTEAIDALNKASSENTQNIAKNSNHIKTLENNIEEGLLELSGHLIDQKADLTKDIKTLENNIEEGLLELSGHLIDQKADLTKDIKALESNVEEGLLDLSGRLLDQKADIAQNQANIQDLAAYNELQDQYAQKQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDTNKASADTKFAATADAITKNGNAITKNAKSITDLGTKVDGFDGRVTALDTKVNALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 33 - protein, 587 aa, Moraxalla catarrhalis
MKTMKLLPLKIAVTSALIVGLGAASTANAQLVERFFPNIFLDKPLAKQHYHNVVVGDTSIVSDLQSNSDQLKFYSDDEGLVPDSLLFNKMLHEQLLNGFKEGDTIIPLDENGKPVYKVDYKLDGKEPRKVYSVTTKIATAEDVATSSYANGIQKDIDDLYDFDHQVTERLTQHGKTIYRNGERILANEESVQYLNKEVQNNIEHIYELAQQQDQHSSDIKTLESNVEKGLLELSGHLIDQKADLTKDIKTLENNVEEGLLDLSGRLIDQKADIAQNQANIQDLAAYNELQDQYAQKQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDTNKASADTKFAATADAITKNGNAITKNAKSITDLGTKVDGFDSRVTALDTKVNALDTKVNALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 34 - protein, 678 aa, Moraxalla catarrhalis; identical to SEQ ID NO: 32

SEQ ID NO: 35 - protein, 616 aa, Moraxalla catarrhalis
MKTMKLLPLKIAVTSALIVGLGAASTANAQVRDKSLEDIEALLGKIDISKLEKEKKQQTELQKYLLLSQYANVLTMEELNKNVEKNTNSIEALGYEIGWLENDIADLEEGVEELTKNQNTLIEKDEEHDRLIAQNQADIKTLENNVVEELFNLSDRLIDQEADIAKNNASIEELYDFDNEVAERIGEIHAYTEEVNKTLEKLITNSVKNTDNIDKNKADIQALENNVEEGLLELSGHLIDQKADLTKDIKALESNVEEGLLDLSGRLLDQKADIAKNQADIAQNQTDIQDLAAYNELQDQYAQKQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKVSAANTDRIAKNKADADASFETLTKNQNTLIEKDKEHDKLITANKTAIDANKASADTKFAATADAITKNGNAITKNAKSITDLGTKVDGFDSRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 36 - protein, 676 aa, Moraxalla catarrhalis
MKTMKLLPLKIAVTSALIVGLGAASTANAQATETFLPNLFDNDYTETTDPLYHGMILGNTAITQDTQYKFYAENGNEVPDSLFFNKILHDQQLNGFKEGDTIIPLDENGKPVYKLDEITENGVKRKVYSVTTKTATREDVEQSAYSRGIQGDIDDLYEANKENVNRLIEHGDKIFANEESVQYLNKEVQNNIENIHELAQQQDQHSSDIKTLKKNVEEGLLELSGHLIDQKADLTKDIKALESNVEEGLLDLSGHLIDQKADLTKDIKALESNVEEGLLDLSGRLLDQKADIAKNQADIAQNQTDIQDLAAYNELQDQYAQKQTEAIDALNKASSENTQNIEDLAAYNELQDQYAQKQTEAIDALNKASSENTQNIEDLAAYNELQDQYAQKQTEAIDALNKASSENTQNIEDLAAYNELQDQYAQKQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDGRVTALDTKVNALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 37 - protein, 629 aa, Moraxalla catarrhalis
MKTMKLLPLKIAVTSALIVGLGAASTANAQNGTSTKLKNLKEYAQYLDNYAQYLDDDIDDLDKEVGELSQNIAKNQANIKDLNKKLSRDIDSLREDVYDNQYEIVNNQADIEKNQDDIKELENNVGKELLNLSGRLLDQKADIDNNINNIYELAQQQDQHSSDIKTLKKNVEEGLLELSGHLIDQKSDIAQNQTDIQDLATYNELQDQYAQKQTEAIDALNKASSENTQNIEDLAAYNELQDAYAKQQTEAIDALNKASSENTQNIQDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDAFDGRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 38 - protein, 683 aa, Moraxalla catarrhalis
MKTMKLLPLKIAVTSAMIVGLGMASTANAQQQRSPKTETFLPNIFFNEYADDLDTLYHNMILGDTAITHDDQYKFYADDATEVPDSLFFNKILHDQLLYGFKEGDKIIPLDENGKPVYKLDKRLDNGVQKTVYSVTTKTATADDVNSAYSRGIQGDIDDLYEANKENVNRLIEHGDKIFANEESVQYLNKEVQNNIENIHELAQQQDQHSSDIKTLKKNVEEGLLELSGHLIDQKTDIAQNQTDIQDLATYNELQDQYAQKQTEAIDALNKASSENTQNIAKNSNRIKALENNIEEGLLELSGHLIDQKADLTKDIKALESNVEEGLLDLSGRLIDQKADIAQNQANIQDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDGRVTALDTKVNALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 39 - protein, 700 aa, Moraxalla catarrhalis
MKTMKLLPLKIAVTSAMIVGLGAASTANAQAQSNRSLDQVQALLRGIDETKIKKEIQQSQQPELNKYLTFNQLANALNIEELNNNVQKNTQRLDSAATLYGDLSKTVPKSIKENKESIKENKESIKENKESIKENKESIKENKESIKENKESITTLTRKSFQNQVDIVRNNASIEDLYAYGQEVAKSIGEIHAYTEEVNKTLENLITNSVENTNNITKNKADIQALENNVVEELFNLSGRLIDQKADIDNNINNIYELAQQQDQHSSDIKTLKKNVEEGLLELSGHLIDQKADLTKDIKTLESNVEEGLLDLSGRLLDQKADIAQNQANIQDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTVIDANKASADTKFAATADAITKNGNAITKNAKSITDLGTKVDGFDGRVTALDTKVNALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 40 - protein, 676 aa, Moraxalla catarrhalis
MKTMKLLPLKIAVTSALIVGLGAASTANAQATETFLPNLFDNDYIETTDPLYHGMILGNTAITQDTQYKFYAENGNEVPDSLFFNKILHDQQLNGFKEGDTIIPLDENGKPVYKLDEITENGVKRKVYSVTTKTATREDVEQSAYSRGIQGDIDDLYEANKENVNRLIEHGDKIFANEESVQYLNKEVQNNIENIHELAQQQDQHSSDIKTLKKNVEEGLLELSGHLIDQKADLTKDIKTLENNVEEGLLELSGHLIDQKADLTKDIKALESNVEEGLLDLSGRLLDQKADIAKNQADIAQNQTDIQDLAAYNELQDQYAQKQTEAIDALNKASSENTQNIEDLAAYNELQDQYAQKQTEAIDALNKASSENTQNIEDLAAYNELQDQYAQKQTEAIDALNKASSENTQNIEDLAAYNELQDQYAQKQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDGRVTALDTKVNALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 41 - protein, 678 aa, Moraxalla catarrhalis
MKTMKLLPLKIAVTSAMIIGLGAASTANAQLAEQFFPNIFSNHAPVKQHYHNVVVGDTSIVENLQDSDDTQLKFYSNDEYSVPDSLLFNKMLHEQQLNGFKKGDTIIPLDENGKPVYKVDYKLDGQEPRRVYSVTTKIATQDDVDNSPYSRGIQGDIDDLYEANKENVNRLIEHGDKIFANEESVQYLNKEVQNNIENIYELAQQQDQHSSDIKTLKKNVEEGLLELSGRLIDQKADIAQNQANIQDLAAYNELQDQYAQKQTEAIDALNKASSENTQNIEDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKVSAANTDRIAKNKADADASFETLTKNQNTLIEKDKEHDKLITANKTAIDANKASADTKFAATADAITKNGNAITKNAKSITDLGTKVDGFDGRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 42 - protein, 613 aa, Moraxalla catarrhalis; identical to SEQ ID NO: 22

SEQ ID NO: 43 - protein, 589 aa, Moraxalla catarrhalis
MKTMKLLPLKIAVTSALIVGLGAASTANAQATNKDITLEDVLKSIEEIDPYELRDYIEYPTAIERFLLLSQYGNTLTLEEFDNDIELLDQDVEDLEESVTELAKNQNSLIEQGEAIKEDLQGLADFVERQEDKILQNETSIKKNTQRNLVNGFEIEKNKDAIAKNNESIEDLYDFGHEVAKSIGEIHAHNEAQNETLKDLITNSVKNTDNITKNKADIQALESNVEKGLLELSGHLIDQKADIDNNINNIHELAQQQDQHSSDIKTLKKNVEEGLLELSGHLIDQKSDIAQNQANIQDLATYNELQDQYAQKQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDSRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 44 - protein, 684 aa, Moraxalla catarrhalis
MKTMKLLPLKIAVTSAMMVGLGMASTANAQQQKSPKTEIFLPNLFDNDNTELTDPLYHNMILGNTALLTQENQYKFYADDGNGVPDSLLFNKILHDQLLHGFKEGDTIIPLDENGKPVYKLDSIVEQGKTKTVYSVTTKTATADDVNSAYSRGIQGDIDDLYEANKENVNRLIEHGDKIFANEESVQYLNREVQNNIENIHELAQQQDQHSSDIKTLKKNVEKDLLDLSGRLIAQKEDIAQNQTDIQDLATYNELQDQYAQKQTEAIDALNKASSENTQNIAKNSNHIKTLENNIEEGLLELSGHLIDQKADLTKDIKALESNVEEGLLDLSGRLIDQKADIAQNQANIQDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDGRVTALDTKVNALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 45 - protein, 650 aa, Moraxalla catarrhalis
MKTMKLLPLKIAVTSALIVGLGAASTANAQQQQKTKTEVFLPNLFYNDYIEETDLLYHNMILGDTAALVDRQNYSNSQLKFYSNDEESVPDSLLFSKMLNNQQLNGFKAGDIIIPVDANGQVIYQKDTRVEGGKTRTVLSVTTKIATQQDVDSAYSRGIQGKVNDLDDEMNFLNHDITSLYDVTANQQDDIKGLKKGVKDLKKGVKGLNKELKELDKEVGVLSRDIGSLNDDVAQNNESIEDLYDFSQEVADSIGEIHAHNKAQNETLQDLITNSVENTNNITKNKADIQALENNVVEELFNLSGRLIDQKADLTKDIKTLESNVEEGLLELSGHLIDQKADIAKNQADIAQNQANIQDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDENKASADTKFAATADAITKNGNAITKNAKSITDLGTKVDGFDGRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 46 - protein, 616 aa, Moraxalla catarrhalis
MKTMKLLPLKIAVTSALIVGLGAVSTTNAQAQSRSLDQIQTKLADLAGKIAAGKNGGGQNNQNNQNDINKYLFLSQYANILTMEELNNNVVKNSSSIETLETDFGWLENDVADLEDGVEELTKNQNTLIEKDEEHDRLIAQNQADIQTLENNVVEELFNLSDRLIDQKADIAKNQADIAQNNESIEELYDFDNEVAEKIGEIHAYTEEVNKTLQDLITNSVKNTDNIDKNKADIDNNINHIYELAQQQDQHSSDIKTLKNNVEEGLLELSGHLIDQKADLTKDIKTLENNVEEGLLDLSGRLIDQKADIAKNQADIAQNQTDIQDLAAYNELQDQYAQKQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDENKASADTKFAATADAITKNGNAITKNAKSITDLGTKVDGFDGRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 47 - protein, 668 aa, Moraxalla catarrhalis
MKTMKLLPLKIAVTSALIVGLGTASTANAQVASPANQKIQQKIKKVRKELRQDIKSLRNDIDSNTADIGSLNDDVADNQDDILDNQADIAKNQDDIEKNQADIKELDKEVGVLSREIGSLNDDIADNYTDIIDNYTDIIDNQANIAKNQDDIEKNQADIKELDKEVGVLSREIGSLNDDVADNQDDIAKNQADIQTLENNVEEGLLELSGHLLDQKADIDNNINNIYELAQQQDQHSSDIKTLKKNVEEGLLELSGHLIDQKTDIAQNQANIQDLATYNELQDQYAQEQTEAIDALNKASSENTQNIAKNSNRIKALESNVEEGLLELSGHLIDQKADLTKDIKALESNVEEGLLELSGHLIDQKADIAQNQANIQDLAAYNELQDQYAQKQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKVSADTKFAATADAITKNGNAITKNAKSITDLGTKVDAFDSRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 48 - protein, 674 aa, Moraxalla catarrhalis
MKTMKLLPLKIAVTSAMIVGLGATSTVNAQVVEQFFPNIFFNENHDELDDAYHNMILGDTAIVSNSQDNSTQLKFYSNDEDSVPDSLLFSKLLHEQQLNGFKAGDTIIPLDKDGKPVYTKDTRTKDGKVETVYSVTTKIATQDDVEQSAYSRGIQGDIDDLYDINREVNEYLKATHDYNERQTEAIDALNKASSANTDRIDTAEERIDKNEYDIKALESNVEEGLLELSGHLIDQKADLTKDIKALESNVEEGLLELSGHLIDQKADLTKDIKALESNVEEGLLDLSGRLIDQKADIAQNQANIQDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDGRVTALDTKVNALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 49 - protein, 613 aa, Moraxalla catarrhalis
MKTMKLLPLKIAVTSAMIIGLGAASTANAQSRDRSLEDIQDSISKLVQDDIDTLKQDQQKMNKYLLLNQLANTLITDELNNNVIKNTNSIEALGDEIGWLENDIADLEEGVEELTKNQNTLIEKDEEHDRLIAQNQADIQTLENNVVEELFNLSGRLIDQEADIAKNNASIEELYDFDNEVAERIGEIHAYTEEVNKTLENLITNSVKNTDNIDKNKADIDNNINHIYELAQQQDQHSSDIKTLKNNVEEGLLELSGHLIDQKADLTKDIKALESNVEEGLLDLSGRLLDQKADLTKDIKALESNVEEGLLDLSGRLLDQKADIAQNQTDIQDLAAYNELQDQYAQKQTEAIDALNKASSENTQNIEDLAAYNELQDAYAKQQTEAIDALNKASSENTQNIAKNQADIANNINNIYELAQQQDQHSSDIKTLAKASAANTNRIATAELGIAENKKDAQIAKAQANANKTAIDENKASADTKFAATADAITKNGNAITKNAKSITDLGTKVDGFDGRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 50 - protein, 576 aa, Moraxalla catarrhalis
MKTMKLLPLKIAVTSAMIVGLGATSTVNAQVVEQFFPNIFFNENHDELDDAYHNMILGDTAIVSNSQDNSTQLKFYSNDEDSVPDSLLFSKLLHEQQLNGFKAGDTIIPLDKDGKPVYTKDTRTKDGKVETVYSVTTKIATQDDVEQSAYSRGIQGDIDDLYDINREVNEYLKATHDYNERQTEAIDALNKASSANTDRIDTAEERIDKNEYDIKALESNVEEGLLELSGHLIDQKADLTKDIKALESNVEEGLLELSGHLIDQKADLTKDIKALESNVEEGLLDLSGRLLDQKADIAKNQADIAQNQTDIQDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDGRVTALDTKVNALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 51 - protein, 684 aa, Moraxalla catarrhalis; identical to SEQ ID NO: 44

SEQ ID NO: 52 - protein, 686 aa, Moraxalla catarrhalis
MKTMKLLPLKIAVTSAMIIGLGAASTANAQATETFLPNLFDNDYTETTDPLYHGMILGNTAITQDTQYKFYAENGNEVPDSLFFNKILHDQQLNGFKEGDTIIPLDENGKPVYKLDEITENGVKRKVYSVTTKTATREDVEQSAYSRGIQGDIDDLYEANKENVNRLIEHGDKIFANEESVQYLNKEVQNNIENIHELAQQQDQHSSDIKTLKKNVEEGLLELSGRLIAQKEDIAQNQTDIQDLATYNELQDQYAQKQTEAIDALNKASSENTQNIAKNSNHIKTLENNIEEGLLELSGHLIDQKADLTKDIKALESNVEEGLLDLSGRLLDQKADIAKNQADIAQNQTDIQDLAAYNELQDQYAQKQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDGRVTALDTKVNALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 53 - protein, 630 aa, Moraxalla catarrhalis; identical to SEQ ID NO: 10

SEQ ID NO: 54 - protein, 616 aa, Moraxalla catarrhalis
MKTMKLLPLKIAVTSALIVGLGAASTANAQAQDRSLEQIQDKLANLVEKIEQAKSQNGQSQKDINQYLLLSQYANVLTMEELNNNVVKNSSSIETLDNDIAWLNDDLIDLDKEVGVLSRDIGSLHDDVAQNQADIKTLKNNVVEELFNLSDRLIDQEADIAQNNESIEDLYDFGREVAESIGEIHAHNEAQNETLKDLITNSVKNTDNITKNKADIQALENDVGKELLNLSGRLIDQKADIDNNINHIYELAQQQDQHSSDIKTLKNNVEEGLLELSGHLIDQKADLTKDIKALESNVEEGLLDLSGRLLDQKADIAQNQANIQDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDSRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 55 - protein, 614 aa, Moraxalla catarrhalis
MKTMKLLPLKIAVTSAMIIGLGATSTVNAQVVEQFFPNIFFNENHDELDDAYHNMILGDTAIVSNSQDNSTQLKFYSNDEDSVPDSLLFSKLLHEQQLNGFKAGDTIIPLDKDGKPVYTKDTRTKDGKVETVYSVTTKIATQDDVEQSAYSRGIQGDIDDLYDINREVNEYLKATHDYNERQTEAIDALNKASSANTDRIDTAEERIDKNEYDIKALESNVGKDLLDLSGRLIAQKEDIDNNINHIYELAQQQDQHSSDIKTLKNNVEEGLLELSGHLIDQKADLTKDIKTLESNVEEGLLDLSGRLIDQKADIAQNQANIQDLAAYNELQDQYAQKQTEAIDALNKASSENTQNIEDLAAYNELQDAYAKQQTEAIDALNKASSENTQNIAKNQADIANNINNIYELAQQQDQHSSDIKTLAKVSAANTDRIAKNKADADASFETLTKNQNTLIEKDKEHDKLITANKTAIDANKASADTKFAATADAITKNGNAITKNAKSITDLGTKVDGFDGRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 56 - protein, 679 aa, Moraxalla catarrhalis
MKTMKLPPLKIAVTSAMIIGLGAASTANAQTTETFLPNLFDNDYTETTDPLYHGMILGDTAITQDTQYKFYAENGNEVPDSLFFNKILHDQLLNGFKAGDTIIPLDENGKPVYKLDERTENGVKRKVYSVTTKTATQADVEQSAYSRGIQGDIDDLYEANKENVNRLIEHGDKIFANEESVQYLNREVQNNIENIHELAQQQDQHSSDIKTLKKNVEKDLLDLSGRLIAQKEDIAQNQTDIQDLATYNELQDQYAQKQTEAIDALNKASSENTQNIAKNSNHIKTLENNIEECLLELSGHLIDQKADLTKDIKALESNVEEGLLDLSGRLIDQKADIAQNQANIQDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDGRVTALDTKVNALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 57 - protein, 724 aa, Moraxalla catarrhalis
MKTMKLLPLKIAVTSALIVGLGAASTANAQETLEEVLESIKQINEQDLQDDIGYNSALDRYLVLSQYGNLLIAKELNENVEKNSNSIAKNSNSIADLEADVGYLAENQNTLIEQNETINQELEGITHELESFIAYAHAQDQKNLVNEFEIEKNKDAIAKNNESIEDLYDFGHEVAESIGEIHAYTEEVNKTLENLITNSVKNTDNITKNKADIQALESNVEKELLNLSGRLIDQKADIDNNINHIYELAQQQDQHSSDIKTLKKNVEEGLLELSGHLIDQKSDIAQNQTDIQDLATYNELQDQYAQKQTEAIDALNKASSENTQNIEDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDSRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 58 - protein, 611 aa, Moraxalla catarrhalis
MKTMKLLPLKIAVTSALIVGLGAASTANAQAQARDRSLEDIQALIGNIDVDKIRSQKQKNPEIFQYLLLNQLSNTLITDELNNNVIKNTNSIETLDNDIAWLNDDLIDLDKEVGVLSRDIGSLHDDVAQNQADIKTLENNVVEELFNLSDRLIDQEAEIAQNNESIEDLYDFGREVAESIGEIHAHNEAQNETLKDLITNSVKNTDNIDKNKADIQALENNVEEGLLELSGHLIDQKADLTKDIKALESNVEEGLLDLSGRLLDQKADIAKNQADIAQNQTDIQDLAAYNELQDQYAQKQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKVSAANTDRIAKNKADADASFETLTKNQNTLIEKDKEHDKLITANKTAIDANKASADTKFAATADAITKNGNAITKNAKSITDLGTKVDGFDSRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF